(19) United States
(12) Patent Application Publication
(10) Pub. No.: US 2025/0362300 A1
(43) Pub. Date: Nov. 27, 2025

(54) NOVEL BIOMARKERS FOR DIAGNOSING PANCREATIC CANCER

(71) Applicant: BERTIS INC, Gwacheon-si (KR)

(72) Inventors: Dongyoung Noh, Seoul (KR); Seungman Han, Seoul (KR); Myeong-Hee Yu, Seoul (KR); Un Beom Kang, Suji-Gu (KR); Sung Hyun Huh, Seoul (KR); Hyeonji Kim, Suji-Gu (KR); Jin-Young Jang, Seoul (KR); Youngmin Han, Seoul (KR)

(21) Appl. No.: 18/776,094

(22) Filed: Jul. 17, 2024

(30) Foreign Application Priority Data
May 22, 2024 (KR) 10-2024-0066462

(51) Int. Cl. G01N 33/574; C12Q 1/6886

(52) U.S. Cl. CPC G01N 33/57438; C12Q 1/6886; C12Q 2600/136; C12Q 2600/158

(57) ABSTRACT

The present invention relates to a method capable of predicting the onset of pancreatic cancer with high accuracy by measuring the expression levels of genes or proteins involved in the onset of pancreatic cancer. The present invention discovers effective biomarkers for pancreatic cancer, especially pancreatic ductal adenocarcinoma, and thus provides a multifaceted, comprehensive and novel therapeutic strategy for early diagnosis of pancreatic cancer, including a method capable of predicting the onset of pancreatic cancer at an early stage with high reliability. Ultimately, the present invention may be advantageously used to improve the survival rate against pancreatic cancer.

### TECHNICAL FIELD

The present invention relates to a method capable of predicting the onset of pancreatic cancer at an early stage with high accuracy by measuring the expression levels of factors that are specifically regulated in pancreatic cancer.

### BACKGROUND ART

Pancreatic cancer is the seventh leading cause of cancer-related death worldwide, and is the second to fifth leading cause, especially in developed countries. Although pancreatic cancer is a major cause of cancer-related death as described above, it is known that early detection and diagnosis of pancreatic cancer are still difficult because symptoms thereof are generally not noticeable in the early stages, detection thereof in the early stages is difficult, and studies on specific tumor markers are insufficient. Due to the absence of such early diagnosis methods, only 5 to 22% of actual pancreatic cancer patients are diagnosed early enough to undergo tumor resection, and pancreatic cancer is one of the most lethal types of cancer, with a 5-year survival rate of only 12.6% (2018 National Cancer Registration Statistics).

Currently, for the diagnosis of pancreatic cancer, imaging techniques such as ultrasound, CT imaging, MRI, hematography, endoscopic retrograde cholangiopancreatography, and ultrasound endoscopy are usually used clinically, and tumor markers (biomarkers) are also used. Pancreatic cancer biomarker known to date, whose diagnostic effects have been confirmed, include carbohydrate antigen 19-9 (CA19-9), which has a limitation in that it does not have sufficient sensitivity or specificity to be effectively used in the early diagnosis of pancreatic cancer. Although it is true that CA19-9 is a useful tumor marker in predicting the prognosis of pancreatic cancer and tracking the treatment process of pancreatic cancer in clinical practice, CA19-9 is generally evaluated to be less useful as a screening test for pancreatic cancer due to the low incidence of pancreatic cancer. Therefore, in order to increase the survival rate of pancreatic cancer patients, there is an urgent need to discover novel pancreatic cancer-specific biomarkers that enable early diagnosis of pancreatic cancer with higher accuracy.

Meanwhile, blood is a biological sample that is attracting attention in cancer biomarker research due to the minimally invasive characteristics of liquid biopsy. Accordingly, the present inventors have compared and analyzed serum samples from pancreatic cancer patients and control groups to discover pancreatic cancer-specific biomarkers that are efficient for diagnosing pancreatic cancer, and have sought to discover biomarkers capable of predicting or diagnosing pancreatic cancer at an early stage with high accuracy and evaluate the effects thereof.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### PRIOR ART DOCUMENTS

### Patent Documents

Patent Document 1. US Patent Application Publication No. 2013-0280166

### DISCLOSURE

### Technical Problem

The present inventors have made extensive research efforts to discover efficient diagnostic biomarkers for pancreatic cancer, which is a major cause of cancer-related death worldwide, but has a very low patient survival rate due to the absence of a highly accurate early diagnosis method, thereby developing a novel diagnosis method capable of significantly lowering the mortality rate for pancreatic cancer, especially pancreatic ductal adenocarcinoma. As a result, the present inventors have discovered proteins or genes encoding the proteins whose expression is specifically regulated in pancreatic cancer patients, and have found that these proteins or genes are highly reliable markers that are capable of diagnosing pancreatic cancer at an early stage with high accuracy by measuring the expression levels of these markers, and may also be applied to mass spectrometry-based diagnostic methods using blood, thereby completing the present invention.

Therefore, an object of the present invention is to provide a composition for diagnosing pancreatic cancer or a diagnostic kit comprising the same.

Another object of the present invention is to provide a method of providing information necessary for diagnosis of pancreatic cancer.

Still another object of the present invention is to provide a method for screening a composition for preventing or treating pancreatic cancer.

Yet another object of the present invention is to provide a system for diagnosing pancreatic cancer.

Other objects and advantages of the present invention will be more apparent from the following detailed description, the appended claims and the accompanying drawings.

### Technical Solution

According to one aspect of the present invention, the present invention provides a composition for diagnosing pancreatic cancer, comprising, as an active ingredient, an agent for measuring the expression level of at least one polypeptide selected from the group consisting of ANPEP (aminopeptidase N), APOA4 (apolipoprotein A-IV), APOC3 (apolipoprotein C-III), C9 (complement component C9), CRP (C-reactive protein), HGFAC (hepatocyte growth factor activator), IGFBP2 (insulin-like growth factor-binding protein 2), ITIH3 (inter-alpha-trypsin inhibitor heavy chain H3), LRG1 (leucine-rich alpha-2-glycoprotein), ORM1 (alpha-1-acid glycoprotein 1), PFN1 (profilin-1), PIGR (polymeric immunoglobulin receptor), PON3 (serum paraoxonase/lactonase 3), SERPINA3 (alpha-1-antichymotrypsin), and VWF (von Willebrand factor), or a fragment thereof, or a gene encoding the polypeptide or fragment thereof.

The present inventors have made extensive research efforts to discover efficient diagnostic biomarkers for pancreatic cancer, which is a major cause of cancer-related death worldwide, but has a very low patient survival rate due to the absence of a highly accurate early diagnosis method, thereby developing a novel diagnosis method capable of significantly lowering the mortality rate for pancreatic cancer, especially pancreatic ductal adenocarcinoma. As a result, the present inventors have discovered proteins or genes encoding the proteins whose expression is specifically regulated in pancreatic cancer patients, and have found that these proteins or genes are highly reliable markers that are capable of diagnosing pancreatic cancer at an early stage with high accuracy by measuring the expression levels of these markers, and may also be applied to mass spectrometry-based diagnostic methods using blood, thereby completing the present invention.

In the present specification, all genes used for the diagnosis or early diagnosis of pancreatic cancer may be used independently or in combination of two or more as biomarkers for the diagnosis of pancreatic cancer, and when they are used in combination, the set of the genes may be a “biomarker panel”.

In the present specification, the term “biomarker panel” may also be referred to as “biomarker detection panel”, and means a set of at least two biomarkers that may be used for the detection, diagnosis, determination of prognosis, staging, or monitoring of a disease or condition. The biomarker components of this biomarker set may be physically associated, such as by being packaged together, or by being reversibly or irreversibly bound to a solid support. For example, the biomarker panel of the present invention may be provided, in separate tubes that are sold or shipped together, for example as part of a kit, or can be provided on a chip, membrane, strip, filter, or beads, particles, filaments, fibers, or other supports, in or on a gel or matrix, or bound to the wells of a multi-well plate. However, the biomarker panel of the present invention is not limited to the above examples and includes any type of biomarker panel that may be used as a combination of biomarkers for diagnosis of diseases.

In the present invention, the “diagnosis” or “diagnosing” includes: determining the susceptibility of a subject to a specific disease or disorder; determining whether or not a subject currently has a particular disease or disorder; determining the prognosis of a subject with a specific disease or disorder (e.g., identification of pre-metastatic or metastatic cancer conditions, determination of cancer stages, or determination of responsiveness of cancer to therapy); or therametrics (e.g., monitoring states of a subject to provide information about treatment effects). With regard to the purposes of the present invention, the diagnosis or diagnosing refers to determining whether or not the above-described cancer has developed or the likelihood (risk) of developing the cancer.

In the present specification, the term “composition for diagnosing” refers to an integrated mixture or device including a means for measuring the expression level of at least one gene selected from the group consisting of ANPEP, APOA4, APOC3, C9, CRP, HGFAC, IGFBP2, ITIH3, LRG1, ORM1, PFN1, PIGR, PON3, SERPINA3, and VWF, or a protein encoded thereby, in order to determine whether or not a subject has developed pancreatic cancer or to predict the likelihood of developing pancreatic cancer in a subject. Therefore, the term may also be expressed as a “kit for diagnosing”.

According to a specific embodiment of the present invention, the fragment of the ANPEP polypeptide has the amino acid sequence of SEQ ID NO: 1 (ALEQALEK);
the fragment of the APOA4 polypeptide has the amino acid sequence of SEQ ID NO: 2 (LTPYADEFK);
the fragment of the APOC3 polypeptide has the amino acid sequence of SEQ ID NO: 3 (GWVTDGFSSLK);
the fragment of the C9 polypeptide has the amino acid sequence of SEQ ID NO: 4 (ALPTTYEK);
the fragment of the CRP polypeptide has the amino acid sequence of SEQ ID NO: 5 (ESDTSYVSLK);
the fragment of the HGFAC polypeptide has the amino acid sequence of SEQ ID NO: 6 (EALVPLVADHK);
the fragment of the IGFBP2 polypeptide has the amino acid sequence of SEQ ID NO: 7 (LIQGAPTIR);
the fragment of the ITIH3 polypeptide has the amino acid sequence of SEQ ID NO: 8 (ALDLSLK);
the fragment of the LRG1 polypeptide has the amino acid sequence of SEQ ID NO: 9 (LHLEGNK);
the fragment of the ORM1 polypeptide has the amino acid sequence of SEQ ID NO: 10 (SDVVYTDWK);
the fragment of the PFN1 polypeptide has the amino acid sequence of SEQ ID NO: 11 (DSPSVWAAVPGK);
the fragment of the PIGR polypeptide has the amino acid sequence of SEQ ID NO: 12 (VYTVDLGR);
the fragment of the PON3 polypeptide has the amino acid sequence of SEQ ID NO: 13 (YVYVADVAAK);
the fragment of the SERPINA3 polypeptide has the amino acid sequence of SEQ ID NO: 14 (EIGELYLPK); and
the fragment of the VWF polypeptide has the amino acid sequence of SEQ ID NO: 15 (ILAGPAGDSNVVK).

According to a specific embodiment of the present invention, a subject with pancreatic cancer has an increased expression level of at least one gene selected from the group consisting of ANPEP, APOA4, APOC3, C9, CRP, HGFAC, IGFBP2, ITIH3, LRG1, ORM1, PFN1, PIGR, PON3, SERPINA3, and VWF, or a protein encoded thereby.

In the present invention, the term “increased expression level” as used while referring to “the composition for diagnosing pancreatic cancer” means that the expression level of the gene or the protein encoded by the gene is significantly higher than that in a control group or a normal group. Specifically, the term means that the expression level increased by about 10% or more, about 20% or more, about 30% or more, about 40% or more, about 50% or more, or about 60% or more compared to that in the control group or normal group, without being limited thereto.

In the present invention, the term “decreased expression level” means that the expression level of the gene or the protein encoded by the gene is significantly lower than that in a control group or a normal group. Specifically, the phrase means that the expression level decreased by about 10% or more, about 20% or more, about 30% or more, about 40% or more, about 50% or more, or about 60% or more compared to that in the control group or normal group, without being limited thereto.

According to a specific embodiment of the present invention, the pancreatic cancer is pancreatic ductal adenocarcinoma (PDAC).

According to a specific embodiment of the present invention, the agent for measuring the expression level of the polypeptide comprises at least one selected from the group consisting of an antibody, an antigen-binding fragment, a ligand, a peptide nucleic acid (PNA), and an aptamer, which bind specifically to the polypeptide or fragment thereof.

In the present specification, the term “antibody” refers to a substance that binds specifically to an antigen, causing an antigen-antibody reaction. Specifically, with regard to the purposes of the present invention, the antibody may refer to antibodies that bind specifically to the polypeptides mentioned in the present invention.

The antibodies of the present invention include all polyclonal antibodies, monoclonal antibodies, and recombinant antibodies. The antibodies may be easily produced using techniques well known in the art. For example, the polyclonal antibody may be produced by a method well known in the art, which comprises a process of injecting the protein antigen into an animal, collecting blood from the animal, and isolating serum containing the antibody. This polyclonal antibody may be produced from any animal species such as goats, rabbits, sheep, monkeys, horses, pigs, cattle, or dogs. In addition, the monoclonal antibody may be produced using a hybridoma method (see Kohler and Milstein (1976) European Journal of Immunology 6:511-519) well known in the art, or phage antibody library technology (see Clackson et al, Nature, 352:624-628, 1991; Marks et al, J. Mol. Biol., 222:58, 1-597, 1991). The antibody produced by the above method may be isolated and purified using methods such as gel electrophoresis, dialysis, salt precipitation, ion exchange chromatography, and affinity chromatography. In addition, the antibodies of the present invention include functional fragments of antibody molecules as well as complete forms having two full-length light chains and two full-length heavy chains. The “functional fragments of antibody molecules” refers to fragments retaining at least an antigen-binding function, and examples of the functional fragments include Fab, F(ab′), F(ab′)2, and Fv.

In the present specification, the term “antigen-binding fragment” refers to a portion of a polypeptide, to which an antigen can bind, among the entire structure of an immunoglobulin. Examples of the antigen-binding fragment include, but are not limited to, F(ab′)2, Fab′, Fab, Fv, and scFv.

In the present invention, the “peptide nucleic acid (PNA)” refers to an artificially synthesized polymer similar to DNA or RNA, and was first introduced by professors Nielsen, Egholm, Berg and Buchardt (at the University of Copenhagen, Denmark) in 1991. DNA has a phosphate-ribose backbone, whereas PNA has a backbone composed of repeating units of N-(2-aminoethyl)-glycine linked by peptide bonds. Thanks to this structure, PNA has a significantly increased binding affinity for DNA or RNA and a significantly increased stability, and thus is used in molecular biology, diagnostic analysis, and antisense therapy. PNA is disclosed in detail in Nielsen PE, Egholm M, Berg RH, Buchardt O (December 1991). “Sequence-selective recognition of DNA by strand displacement with a thymine-substituted polyamide”. Science 254 (5037): 1497-1500.

In the present invention, the “aptamer” is an oligonucleic acid or peptide molecule, and general contents of the aptamer are disclosed in detail in Bock L C et al., Nature 355 (6360): 5646 (1992); Hoppe-Seyler F, Butz K “Peptide aptamers: powerful new tools for molecular medicine”. J Mol Med. 78 (8): 42630 (2000); Cohen B A, Colas P, Brent R. “An artificial cell-cycle inhibitor isolated from a combinatorial library”. Proc Natl Acad Sci USA. 95 (24): 142727 (1998).

In the present invention, the agent for measuring the expression level of the gene encoding the polypeptide or fragment thereof comprises at least one selected from the group consisting of a primer, a probe, and an antisense oligonucleotide, which bind specifically to the gene.

In the present invention, the “primer” is a fragment that recognizes a target gene sequence, and includes a pair of forward and reverse primers. Specifically, the primer is a primer pair that provides analysis results with specificity and sensitivity. Because the nucleotide sequence of the primer does not match a non-targeted sequence present in a sample, the primer can show high specificity when it amplifies only a target gene sequence containing a complementary primer binding site without causing non-specific amplification.

In the present invention, the “probe” refers to a substance which is capable of binding specifically to the target substance to be detected in a sample and may specifically identify the presence of the target substance in the sample through the binding. The kind of the probe is not specifically limited, as long as it is a substance that is generally used in the art. Specifically, the probe may be peptide nucleic acid (PNA), locked nucleic acid (LNA), a peptide, a polypeptide, a protein, RNA or DNA. Most specifically, the probe is PNA. More specifically, the probe may be a biomaterial derived from an organism, an analogue thereof, or a material produced ex vivo, and examples thereof include enzymes, proteins, antibodies, microorganisms, animal/plant cells and organs, neural cells, DNA, and RNA. Examples of the DNA include cDNA, genomic DNA, and oligonucleotides, examples of the RNA include genomic RNA, mRNA, and oligonucleotides, and examples of the protein include antibodies, antigens, enzymes, and peptides.

In the present invention, the “locked nucleic acid (LNA)” refers to a nucleic acid analog containing a 2′-O or 4′-C methylene bridge [J Weiler, J Hunziker and J Hall Gene Therapy (2006) 13, 496.502]. LNA nucleosides include common nucleic acid bases of DNA and RNA, and can form base pairs according to the Watson-Crick base pairing rule. However, due to ‘locking’ of the molecule attributable to the methylene bridge, the LNA fails to form an ideal shape in the Watson-Crick bond. When the LNA is incorporated in a DNA or RNA oligonucleotide, it can more rapidly pair with a complementary nucleotide chain, thus increasing the stability of the double strand. In the present invention, the “antisense” refers to an oligomer having a sequence of nucleotide bases and a subunit-to-subunit backbone that allows the antisense oligomer to hybridize to a target sequence in an RNA by Watson-Crick base pairing, to form an RNA: oligomer heteroduplex within the target sequence, typically with an mRNA. The oligomer may have exact sequence complementarity to the target sequence or near complementarity.

Since information on the amino acid sequence of the polypeptide according to the present invention and on the nucleic acid sequence encoding the polypeptide is available from various public data sources, those skilled in the art may easily design a primer, a probe, or an antisense oligonucleotide, which bind specifically to the gene encoding the polypeptide, based on the information.

In the present specification, the term “nucleic acid” may also be referred to as the term “nucleic acid molecule”, is meant to encompass DNA (gDNA and cDNA) and RNA molecules. Nucleotides, which are the basic structural units in nucleic acid molecules, include not only natural nucleotides, but also analogues having modified sugar or base moieties (Scheit, Nucleotide Analogs, John Wiley, New York (1980); Uhlman and Peyman, Chemical Reviews, 90:543-584 (1990)).

According to another aspect of the present invention, the present invention provides a diagnostic kit comprising the composition for diagnosing according to the present invention.

In the present invention, it is possible to diagnose the onset, likelihood of onset, responsiveness to therapy, prognosis, stage, likelihood of recurrence, etc. of pancreatic cancer disease using the above diagnostic kit.

In the present invention, the pancreatic cancer to be diagnosed has already been described in detail, and thus detailed description thereof will be omitted below to avoid excessive overlapping.

In the present invention, the kit is an RT-PCR kit, a DNA chip kit, an ELISA kit, a protein chip kit, a rapid kit, or a multiple-reaction monitoring (MRM) kit.

In the present invention, the kit may be, but is not limited to, an RT-PCR kit, a

DNA chip kit, an ELISA kit, a protein chip kit, a rapid kit or a multiple-reaction monitoring (MRM) kit.

The pancreatic cancer diagnostic kit of the present invention may further comprise one or more other component compositions, solutions or devices suitable for analysis methods.

For example, the pancreatic cancer diagnostic kit of the present invention may further comprise essential elements necessary for performing reverse transcription polymerase reaction. The reverse transcription polymerase reaction kit comprises a pair of primers specific to a gene encoding a marker protein. Each primer is an oligonucleotide having a sequence specific to the nucleic acid sequence of the gene, and may have a length of about 7 bp to 50 bp, more preferably about 10 bp to 30 bp. In addition, the kit may comprise primers specific to the nucleic acid sequence of a control gene. In addition, the reverse transcription polymerase reaction kit may comprise a test tube or other suitable container, buffers (having various pHs and magnesium concentrations), deoxynucleotides (dNTPs), enzymes such as Taq-polymerase and reverse transcriptase, DNase and RNase inhibitors, DEPC-water, sterile water, and the like.

In addition, the diagnostic kit of the present invention may comprise essential elements necessary for performing DNA chip assay. The DNA chip kit may comprise a substrate to which a gene or a cDNA or oligonucleotide corresponding to a fragment thereof is attached, and reagents, agents, and enzymes for constructing a fluorescently labeled probe. In addition, the substrate may comprise a control gene or a cDNA or oligonucleotide corresponding to a fragment thereof.

In addition, the diagnostic kit of the present invention may comprise essential elements necessary for performing ELISA. The ELISA kit comprises an antibody specific to the protein. The antibody has high specificity and affinity for the marker protein, with little cross-reactivity to other proteins, and is a monoclonal antibody, a polyclonal antibody, or a recombinant antibody. Furthermore, the ELISA kit may comprise an antibody specific to a control protein. In addition, the ELISA kit may further comprise reagents capable of detecting the bound antibody, for example, a labeled secondary antibody, chromophores, an enzyme (e.g., conjugated with the antibody) and a substrate thereof, or other substances capable of binding to the antibody.

In the diagnostic kit of the present invention, as a fixture for antigen-antibody binding reaction, a well plate synthesized from a nitrocellulose membrane, a PVDF membrane, a polyvinyl resin or a polystyrene resin, or a glass slide made of glass may be used, without being limited thereto.

In addition, in the diagnostic kit of the present invention, a label for the secondary antibody is preferably a conventional chromogenic agent for color development, and examples of the label include, but are not limited to, fluoresceins such as HRP (horseradish peroxidase), alkaline phosphatase, colloid gold, FITC (poly L-lysine-fluorescein isothiocyanate), RITC (rhodamine-B-isothiocyanate), and dyes.

In addition, in the diagnostic kit of the present invention, a chromogenic substrate for inducing color development is preferably selected depending on the label for color development, and may be TMB (3,3′,5,5′-tetramethyl benzidine), ABTS [2,2′-azino-bis (3-ethylbenzothiazoline-6-sulfonic acid)], or OPD (o-phenylenediamine). Here, the chromogenic substrate is more preferably provided as dissolved in buffer (0.1M NaAc, pH 5.5). A chromogenic substrate such as TMB is degraded by HRP, used as a label for the secondary antibody conjugate, to form a chromogen, and the presence of the marker protein is detected by visually checking the degree of deposition of the chromogen.

The washing solution in the diagnostic kit of the present invention preferably comprises phosphate buffer, NaCl and Tween 20. More preferably, the washing solution is a buffer solution (PBST) consisting of 0.02 M phosphate buffer, 0.13 M NaCl, and 0.05% Tween 20. After the antigen-antibody binding reaction, the secondary antibody is allowed to react with the antigen-antibody complex, and then the resulting conjugate is washed 3 to 6 times with a suitable amount of the washing solution added to the fixture. As the reaction stop solution, a sulfuric acid solution (H₂SO₄) is preferably used.

According to another aspect of the present invention, the present invention provides a method of providing information necessary for diagnosis of pancreatic cancer, comprising a step of measuring the expression level of at least one polypeptide selected from the group consisting of ANPEP (aminopeptidase N), APOA4 (apolipoprotein A-IV), APOC3 (apolipoprotein C-III), C9 (complement component C9), CRP (C-reactive protein), HGFAC (hepatocyte growth factor activator), IGFBP2 (insulin-like growth factor-binding protein 2), ITIH3 (inter-alpha-trypsin inhibitor heavy chain H3), LRG1 (leucine-rich alpha-2-glycoprotein), ORM1 (alpha-1-acid glycoprotein 1), PFN1 (profilin-1), PIGR (polymeric immunoglobulin receptor), PON3 (serum paraoxonase/lactonase 3), SERPINA3 (alpha-1-antichymotrypsin), and VWF (von Willebrand factor), or a fragment thereof, or a gene encoding the polypeptide or fragment thereof, in a biological sample isolated from a subject of interest.

In the present invention, the polypeptides whose expression levels are to be measured have already been described in detail, and thus detailed description thereof will be omitted below to avoid excessive overlapping.

The present inventors have found for the first time that the expression levels of ANPEP, C9, CRP, IGFBP2, ITIH3, LRG1, ORM1, PIGR, SERPINA3 and VWF proteins or genes encoding these proteins in patients with pancreatic cancer, especially pancreatic ductal adenocarcinoma, are positively correlated with the likelihood of developing pancreatic cancer, and the expression levels of APOA4, APOC3, HGFAC, PFN1 and PON3 proteins or genes encoding these proteins in the patients are negatively correlated with the likelihood of developing pancreatic cancer. Accordingly, if the above-described positively correlated proteins or genes encoding them are highly expressed in a subject or a biological sample isolated from the subject, or if the above-described negatively correlated proteins or genes encoding them are under-expressed in the subject or the biological sample, the subject is determined to be a subject who has developed pancreatic cancer or is likely to develop pancreatic cancer in the future.

In the present specification, the term “highly expressed” means that the expression level of the gene of interest or the protein encoded by the gene is significantly higher than that in a control group or a normal group. Specifically, the term “highly expressed” means that the expression level increased by about 10% or more, about 20% or more, about 30% or more, about 40% or more, about 50% or more, or about 60% or more compared to that in the control group or the normal group, without being limited thereto.

In the present specification, the term “under-expressed” means that the expression level of the gene of interest or the protein encoded by the gene is significantly lower than that in a control group or a normal group. Specifically, the term “under-expressed” means that the expression level decreased by about 10% or more, about 20% or more, about 30% or more, about 40% or more, about 50% or more, or about 60% or more compared to that in the control group or the normal group, without being limited thereto.

In the present specification, the term “subject” refers to a subject that provides a sample in which the expression level of the gene or the protein encoded thereby of the present invention is to be measured, and that is ultimately to be analyzed as to whether pancreatic cancer has developed. Subjects include, without limitation, humans, mice, rats, guinea pigs, dogs, cats, horses, cows, pigs, monkeys, chimpanzees, baboons, or rhesus monkeys, and are specifically humans. Since the composition of the present invention provides information for not only determining whether pancreatic cancer has occurred but also predicting the genetic risk of developing pancreatic cancer in the future, the subject of the present invention may be a patient with pancreatic cancer or a healthy subject who has not yet developed pancreatic cancer.

In the present invention, the “biological sample” refers to any material, biological fluid, tissue or cells obtained or derived from the subject. For example, the biological sample may include whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid. Specifically, the biological sample may be a liquid biopsy (e.g., patient's tissue, cells, blood, serum, plasma, saliva, sputum or ascites, etc.) collected for histopathological examination by inserting a hollow needle or the like into an in vivo organ without incision of the skin of a patient having a high likelihood of developing cancer.

The method of the present invention may comprise a step of measuring the expression levels of the polypeptides represented by SEQ ID NO: 1 to 15 or genes encoding the same in the biological sample isolated as described above.

In the present invention, the step of measuring the expression level may be a step of measuring the expression level of at least one protein (polypeptide) selected from the group consisting of ANPEP (aminopeptidase N), APOA4 (apolipoprotein A-IV), APOC3 (apolipoprotein C-III), C9 (complement component C9), CRP (C-reactive protein), HGFAC (hepatocyte growth factor activator), IGFBP2 (insulin-like growth factor-binding protein 2), ITIH3 (inter-alpha-trypsin inhibitor heavy chain H3), LRG1 (leucine-rich alpha-2-glycoprotein), ORM1 (alpha-1-acid glycoprotein 1), PFN1 (profilin-1), PIGR (polymeric immunoglobulin receptor), PON3 (serum paraoxonase/lactonase 3), SERPINA3 (alpha-1-antichymotrypsin), and VWF (von Willebrand factor), or a gene encoding the protein.

According to a specific embodiment of the present invention,
the fragment of the ANPEP polypeptide has the amino acid sequence of SEQ ID NO: 1 (ALEQALEK);
the fragment of the APOA4 polypeptide has the amino acid sequence of SEQ ID NO: 2 (LTPYADEFK);
the fragment of the APOC3 polypeptide has the amino acid sequence of SEQ ID NO: 3 (GWVTDGFSSLK);
the fragment of the C9 polypeptide has the amino acid sequence of SEQ ID NO: 4 (ALPTTYEK);
the fragment of the CRP polypeptide has the amino acid sequence of SEQ ID NO: 5 (ESDTSYVSLK);
the fragment of the HGFAC polypeptide has the amino acid sequence of SEQ ID NO: 6 (EALVPLVADHK);
the fragment of the IGFBP2 polypeptide has the amino acid sequence of SEQ ID NO: 7 (LIQGAPTIR);
the fragment of the ITIH3 polypeptide has the amino acid sequence of SEQ ID NO: 8 (ALDLSLK);
the fragment of the LRG1 polypeptide has the amino acid sequence of SEQ ID NO: 9 (LHLEGNK);
the fragment of the ORM1 polypeptide has the amino acid sequence of SEQ ID NO: 10 (SDVVYTDWK);
the fragment of the PFN1 polypeptide has the amino acid sequence of SEQ ID NO: 11 (DSPSVWAAVPGK);
the fragment of the PIGR polypeptide has the amino acid sequence of SEQ ID NO: 12 (VYTVDLGR);
the fragment of the PON3 polypeptide has the amino acid sequence of SEQ ID NO: 13 (YVYVADVAAK);
the fragment of the SERPINA3 polypeptide has the amino acid sequence of SEQ ID NO: 14 (EIGELYLPK); and
the fragment of the VWF polypeptide has the amino acid sequence of SEQ ID NO: 15 (ILAGPAGDSNVVK).

According to a specific embodiment of the present invention, the biological sample is whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid.

In the present specification, the term “blood” means “whole blood”, and such blood includes plasma and serum.

As used in the present specification, the term “whole blood” generally refers to blood composed of uncoagulated plasma and cellular components. Plasma may make up about 50 to 60% of the whole blood volume, and cellular components (e.g., red blood cells, white blood cells, or platelets) may make up about 40 to 50%.

The term “plasma” as used in the present specification refers to the liquid component of blood that functions as a transport medium in supplying nutrients to cells and organs of the body.

The term “serum” as used in the present specification refers to a light yellow liquid separated from blood. Specifically, if blood is left to stand after collection, the fluidity of the blood decreases and a red clot is formed. That is, the term “serum” means a light yellow liquid remaining after removal of the red clot.

According to the present invention, pancreatic cancer may be diagnosed using the biomarkers of the present invention, and the diagnosis may be performed through liquid biopsy using patient-derived body fluids such as blood as a biological sample. This liquid biopsy method has advantages over conventional tissue biopsy procedures in that it may minimize patient's pain due to its non-invasiveness and provide information about cancer more quickly.

According to a specific embodiment of the present invention, the agent for measuring the expression level of the polypeptide comprises at least one selected from the group consisting of an antibody, an oligopeptide, a ligand, a peptide nucleic acid (PNA), and an aptamer, which bind specifically to the polypeptide.

According to a specific embodiment of the present invention, the measurement of the expression level of the polypeptide is performed by protein chip assay, immunoassay, ligand binding assay, MALDI-TOF (matrix assisted laser desorption/Ionization time of flight mass spectrometry) assay, SELDI-TOF (surface enhanced laser desorption/ionization time of flight mass spectrometry) assay, radioimmunoassay, radioimmunodiffusion, Ouchterlony immunodiffusion, rocket immunoelectrophoresis, immunohistochemical staining, complement fixation assay, two-dimensional electrophoresis assay, liquid chromatography-mass spectrometry (LC-MS), LC-MS/MS (liquid chromatography-mass spectrometry/mass spectrometry), Western blotting, or ELISA (enzyme-linked immunosorbent assay).

According to a specific embodiment of the present invention, the measurement of the expression level of the polypeptide is performed by a multiple-reaction monitoring (MRM) method.

In the present invention, the multiple-reaction monitoring method may be performed using mass-spectrometry, specifically triple-quadrupole mass spectrometry.

In the present invention, the multiple-reaction monitoring (MRM) method using mass-spectrometry is an analysis technique capable of monitoring a change in concentration of a specific analyte by selectively isolating, detecting and quantifying the specific analyte. MRM is a method that can quantitatively and accurately measure multiple substances such as trace amounts of biomarkers present in a biological sample. In MRM, mother ions among the ion fragments generated in an ionization source are selectively transmitted to a collision tube by a first mass filter Q1. Then, the mother ions arriving at the collision tube collide with an internal collision gas, and are fragmented to generate daughter ions which are then sent to a second mass filter Q2, where only characteristic ions are transmitted to a detection unit. MRM is an analysis method with high selectivity and sensitivity that can detect only information on a component of interest. MRM is used for quantitative analysis of small molecules and is used to diagnose specific genetic diseases. The MRM method has advantages in that it is easy to simultaneously measure multiple peptides, and it is possible to confirm the relative concentration difference of protein diagnostic marker candidates between a normal person and a cancer patient without using an antibody. In addition, since the MRM analysis method has excellent sensitivity and selectivity, it has been introduced for the analysis of complex proteins and peptides in blood, particularly in proteomic analysis using a mass spectrometer (Anderson L. et al., Mol CellProteomics, 5:375-88, 2006; DeSouza, L. V. et al., Anal. Chem., 81:3462-70, 2009).

In this specification, the term “mass spectrometry” may also be referred to as “mass spectrometry methods” and refers to a method of identifying unknown compounds based on their mass spectra. Mass spectrometry is performed by filtering, detecting, and measuring ions using the m/z value, which is the mass-to-charge ratio. Generally, mass spectrometry includes steps of: (1) charging a compound by ionization; and (2) measuring the molecular weight of the charged compound and calculating the m/z value. The calculated m/z value is used as a reference to identify and quantify the target compound in a complex mixture. The mass spectrometry in the present specification may be performed through any type of mass spectrometry method that uses the above-described principle.

In the present invention, the expression levels of the polypeptides mentioned in the present invention may be measured by the multiple-reaction monitoring method.

In the present invention, to analyze the expression levels of the polypeptides mentioned in the present invention by the multiple-reaction monitoring method, the mass-to-charge ratio values (m/z values) of the target peptides may be used, without being limited thereto.

In the present specification, the term “multiple-reaction monitoring (MRM)” refers to an analysis technique capable of monitoring a change in concentration of a specific analyte by selectively isolating, detecting and quantifying the specific analyte. MRM is a method that can quantitatively and accurately measure multiple substances such as trace amounts of biomarkers present in a biological sample. In MRM, mother ions among the ion fragments generated in an ionization source are selectively transmitted to a collision tube by a first mass filter Q1. Then, the mother ions arriving at the collision tube collide with an internal collision gas, and are fragmented to generate daughter ions which are then sent to a second mass filter Q2, where only characteristic ions are transmitted to a detection unit. MRM is an analysis method with high selectivity and sensitivity that can detect only information on a component of interest. MRM is used for quantitative analysis of small molecules and is used to diagnose specific genetic diseases. The MRM method has advantages in that it is easy to simultaneously measure multiple peptides, and it is possible to confirm the relative concentration difference of protein diagnostic marker candidates between a normal person and a cancer patient without using an antibody. In addition, since the MRM analysis method has excellent sensitivity and selectivity, it has been introduced for the analysis of complex proteins and peptides in blood, particularly in proteomic analysis using a mass spectrometer (Anderson L. et al., Mol CellProteomics, 5:375-88, 2006; DeSouza, L. V. et al., Anal. Chem., 81:3462-70, 2009).

In the present specification, the term “parallel reaction monitoring” refers to the parallel application of MRM. Unlike MRM, which analyzes one pair of mother ions/daughter ions at a time, the parallel reaction monitoring is a method of simultaneously analyzing all daughter ions generated from one selected mother ion.

Mass spectrometry for the proteins used as biomarkers of pancreatic cancer in the present invention may be specifically performed through data-independent acquisition analysis (DIA). In the present specification, the term “data-independent acquisition” refers to a method of analyzing all ions belonging to the m/z values in a selected range without the process of selecting a specific mother ion.

According to a specific embodiment of the present invention,
the mass-to-charge ratio (m/z) of the polypeptide represented by SEQ ID NO: 1, when the z value is 1, is 901.506 or 901.506±1 for a light peptide, and 909.52 or 909.52±1 for a heavy peptide;
the mass-to-charge ratio (m/z) of the polypeptide represented by SEQ ID NO: 2, when the z value is 1, is 1083.536 or 1083.536±1 for a light peptide, and 1091.550 or 1091.550±1 for a heavy peptide;
the mass-to-charge ratio (m/z) of the polypeptide represented by SEQ ID NO: 3, when the z value is 1, is 1196.595 or 1196.595±1 for a light peptide, and 1204.609 or 1204.609±1 for a heavy peptide;
the mass-to-charge ratio (m/z) of the polypeptide represented by SEQ ID NO: 4, when the z value is 1, is 922.496 or 922.496±1 for a light peptide, and 930.508 or 930.508±1 for a heavy peptide;
the mass-to-charge ratio (m/z) of the polypeptide represented by SEQ ID NO: 5, when the z value is 1, is 1128.542 or 1128.542±1 for a light peptide, and 1136.556 or 1136.556±1 for a heavy peptide;
the mass-to-charge ratio (m/z) of the polypeptide represented by SEQ ID NO: 6, when the z value is 1, is 1191.673 or 1191.673±1 for a light peptide, and 1209.708 or 1209.708±1 for a heavy peptide;
the mass-to-charge ratio (m/z) of the polypeptide represented by SEQ ID NO: 7, when the z value is 1, is 968.596 or 968.596±1 for a light peptide, and 978.604 or 978.604±1 for a heavy peptide;
the mass-to-charge ratio (m/z) of the polypeptide represented by SEQ ID NO: 8, when the z value is 1, is 759.468 or 759.468±1 for a light peptide, and 767.482 or 767.482±1 for a heavy peptide;
the mass-to-charge ratio (m/z) of the polypeptide represented by SEQ ID NO: 9, when the z value is 1, is 810.454 or 810.454±1 for a light peptide, and 818.468 or 818.468±1 for a heavy peptide;
the mass-to-charge ratio (m/z) of the polypeptide represented by SEQ ID NO: 10, when the z value is 1, is 1112.526 or 1112.526±1 for a light peptide, and 1120.540 or 1120.540±1 for a heavy peptide;
the mass-to-charge ratio (m/z) of the polypeptide represented by SEQ ID NO: 11, when the z value is 1, is 1213.621 or 1213.621±1 for a light peptide, and 1221.635 or 1221.635±1 for a heavy peptide;
the mass-to-charge ratio (m/z) of the polypeptide represented by SEQ ID NO: 12, when the z value is 1, is 922.506 or 922.506=1 for a light peptide, and 932.508 or 932.508±1 for a heavy peptide;
the mass-to-charge ratio (m/z) of the polypeptide represented by SEQ ID NO: 13, when the z value is 1, is 1098.59 or 1098.59±1 for a light peptide, and 1110.61 or 1110.61±1 for a heavy peptide;
the mass-to-charge ratio (m/z) of the polypeptide represented by SEQ ID NO: 14, when the z value is 1, is 1061.588 or 1061.588±1 for a light peptide, and 1069.602 or 1069.602±1 for a heavy peptide; and
the mass-to-charge ratio (m/z) of the polypeptide represented by SEQ ID NO: 15, when the z value is 1, is 1240.696 or 1240.696±1 for a light peptide, and 1248.71 or 1248.71±1 for a heavy peptide.

In the present invention, the term “heavy peptide” refers to a synthetic peptide labeled with an isotope, etc., and may also be referred to as “synthetic isotopically labeled peptide (SIL). The heavy peptide has a non-radioactive stable isotope linked thereto. As the stable isotope, ¹³C (carbon-13), ¹⁵N (nitrogen-15), ²H (deuterium), or the like is generally used, without being limited thereto. In general, the heavy peptide has the same physiological and chemical characteristics and chemical reactivity as the “light peptide” to be analyzed, and behave differently from the “light peptide” because of the difference in mass due to the isotope. This heavy peptide is used to quantify the absolute amount of the “light peptide” to be analyzed. In the present specification, the term “light peptide” refers to a peptide not labeled with an isotope, as opposed to the “heavy peptide”, and generally refers to a target peptide to be quantitatively analyzed.

According to a specific embodiment of the present invention, the multiple-reaction monitoring method is performed using, as an internal standard substance, either a synthetic peptide obtained by substituting a specific element of a specific amino acid in each of the polypeptides with an isotope, or E. coli beta-galactosidase.

In the present invention, as an internal standard substance, any internal standard substance that is generally used in the multiple-reaction monitoring analysis may be used. For example, E. coli beta-galactosidase may be used.

In addition, in the present invention, in order to measure the absolute amount of the polypeptide in blood, a specific peptide synthesized by substituting a specific amino acid in the target peptide with a stable isotope may be used as an internal standard substance. In this case, the amino acid substituted with the isotope may be lysine or arginine, without being limited thereto. Here, as the synthesized peptide, an isolated peptide with a purity of 95% or higher may be used.

In the present invention, the internal standard substance may comprise at least one radioisotope selected from the group consisting of ²H, ³H, ¹¹C, ¹³C, ¹⁴C, ¹³N, ¹⁵N, ¹⁵O, ¹⁷O and ¹⁸O, but is not limited thereto and may comprise any kind of isotope that may be used as a comparison group to measure the absolute amount of the polypeptide.

According to a specific embodiment of the present invention, the synthetic peptide has the same sequence as the sequence represented by SEQ ID NO: 1, 2, 3, 4, 5, 6, 7, 8, 9, 10, 11, 12, 13, 14, or 15, and contains a stable isotope.

According to a specific embodiment of the present invention, the stable isotope is a stable isotope of any one or more elements selected from the group consisting of carbon and nitrogen.

According to a specific embodiment of the present invention, the measurement of the expression level of the gene encoding the polypeptide is performed by reverse transcription-polymerase chain reaction (RT-PCR), competitive RT-PCR, real-time RT-PCR, RNase protection assay (RPA), Northern blotting, or DNA chip assay.

In the present invention, to measure the presence and expression level of the gene encoding the polypeptide, an analysis method of measuring the mRNA level of the gene may be used. Examples of the analysis method include, but are not limited to, reverse transcription-polymerase chain reaction (RT-PCR), competitive RT-PCR, real-time RT-PCR, RNase protection assay (RPA), Northern blotting, and DNA chip assay.

In the present invention, it is possible to predict responsiveness to therapy to pancreatic cancer by measuring the expression level of the polypeptide or the gene encoding the same in the biological sample isolated from the subject of interest.

In the present invention, it is possible to predict the prognosis of a subject of interest, preferably the prognosis after surgical operation, by measuring the expression level of any one or more of the polypeptides represented by SEQ ID NOs: 1 to 15, or the genes encoding the polypeptides, in the biological sample isolated from the subject of interest. Here, the subject of interest may be a subject who has undergone surgical resection due to pancreatic cancer.

In the present invention, it is possible to determine the stage of pancreatic cancer in the subject of interest by measuring the expression level of the polypeptide or the gene encoding the polypeptide in the biological sample isolated from the subject of interest.

In the present invention, the “stage” refers to the extent to which cancer cells have spread or the stage of cancer progression. The international classification according to the status of cancer progression generally follows the TNM stage classification. Here, ‘T (Tumor Size)’ is a classification according to the size of the primary tumor, ‘N (Lymph Node)’ is a classification according to the degree of lymph node metastasis, and ‘M (Metastasis)’ is a classification according to whether cancer has metastasized to other organs.

In the present invention, it is possible to predict the likelihood of recurrence of pancreatic cancer by measuring the expression level of the polypeptide or the gene encoding the polypeptide in the biological sample isolated from the subject of interest.

According to a specific embodiment of the present invention,
if the measured expression level of the ANPEP (aminopeptidase N), C9 (complement component C9), CRP (C-reactive protein), IGFBP2 (insulin-like growth factor-binding protein 2), ITIH3 (inter-alpha-trypsin inhibitor heavy chain H3), LRG1 (leucine-rich alpha-2-glycoprotein), ORM1 (alpha-1-acid glycoprotein 1), PIGR (polymeric immunoglobulin receptor), SERPINA3 (alpha-1-antichymotrypsin) or VWF (von Willebrand factor) polypeptide or the gene encoding the same in the biological sample isolated from the subject of interest is higher than that in a normal control group, or
if the measured expression level of the APOA4 (apolipoprotein A-IV), APOC3 (apolipoprotein C-III), HGFAC (hepatocyte growth factor activator), PFN1 (profilin-1) or PON3 (serum paraoxonase/lactonase 3) polypeptide or the gene encoding the same in the biological sample is lower than that in the normal control group,
the likelihood of developing the pancreatic cancer is predicted to be high.

According to a specific embodiment of the present invention, the pancreatic cancer is pancreatic ductal adenocarcinoma (PDAC).

According to another aspect of the present invention, the present invention provides a method for screening a composition for preventing or treating pancreatic cancer, comprising steps of:
(a) bringing a test substance into contact with a biological sample containing ANPEP, APOA4, APOC3, C9, CRP, HGFAC, IGFBP2, ITIH3, LRG1, ORM1, PFN1, PIGR, PON3, SERPINA3 and VWF genes, or proteins encoded by the genes, or cells expressing the genes or proteins; and
(b) measuring the expression levels of the proteins or the genes in the biological samples,
wherein, if the activities or expression levels of the ANPEP, C9, CRP, IGFBP2, ITIH3, LRG1, ORM1, PIGR, SERPINA3 and VWF genes or proteins in the biological sample decreased, or
if the activities or expression levels of the APOA4, APOC3, HGFAC, PFN1 and PON3 genes or proteins in the biological sample increased,
the test substance is determined as the composition for preventing or treating pancreatic cancer.

In the present invention, the sample has already been described in detail, and thus detailed description thereof will be omitted to avoid excessive overlapping.

In the present invention, the term “biological sample” refers to any sample containing cells expressing the above-described gene or the protein encoded by the gene, which is obtained from mammals, including humans. Examples of the biological sample include, but are not limited to, tissues, organs, cells, or cell cultures. More specifically, the biological sample may be cancer tissue, cancer cells, a culture thereof, or blood.

The term “test substance” as used while referring to the screening method of the present invention refers to an unknown substance which is used in screening to examine whether it affects the activity or expression level of the gene of the present invention by being added to a sample containing cells expressing the gene. Examples of the test substance include, but are not limited to, compounds, nucleotides, peptides, and natural extracts. The step of measuring the expression level or activity of the gene in the biological sample treated with the test substance may be performed by various expression level and activity measurement methods known in the art.

According to a specific embodiment of the present invention, the pancreatic cancer is pancreatic ductal adenocarcinoma (PDAC).

Since the meaning of the term “pancreatic ductal adenocarcinoma (PDAC)” as used in the present specification has already been described in detail, description thereof will be omitted to avoid excessive overlapping.

According to another aspect of the present invention, the present invention provides a system for diagnosing pancreatic cancer, comprising: an input unit configured to receive an input value; a reading unit comprising a machine learning model pre-trained to read whether pancreatic cancer has occurred; and an output unit configured to output whether pancreatic cancer has occurred, wherein the input value is a measured value for the expression level of at least one polypeptide selected from the group consisting of SEQ ID NO: 1 (ALEQALEK), SEQ ID NO: 2 (LTPYADEFK), SEQ ID NO: 3 (GWVTDGFSSLK), SEQ ID NO: 4 (ALPTTYEK), SEQ ID NO: 5 (ESDTSYVSLK), SEQ ID NO: 6 (EALVPLVADHK), SEQ ID NO: 7 (LIQGAPTIR), SEQ ID NO: 8 (ALDLSLK), SEQ ID NO: 9 (LHLEGNK), SEQ ID NO: 10 (SDVVYTDWK), SEQ ID NO: 11 (DSPSVWAAVPGK), SEQ ID NO: 12 (VYTVDLGR), SEQ ID NO: 13 (YVYVADVAAK), SEQ ID NO: 14 (EIGELYLPK), and SEQ ID NO: 15 (ILAGPAGDSNVVK), in a biological sample.

The term “machine learning” in the present invention refers to algorithms and statistical models that computer systems use to perform tasks without explicit instructions, relying on patterns and inferences. The machine learning model that is used in the present invention may be specifically a deep learning, logistic regression, support vector machine (SVM), random forest, or gradient boosting algorithm GBM) model, more specifically a deep learning model, most preferably a stacking ensemble model including a combination of a total of seven machine learning algorithms to maximize model robustness while providing optimal performance. It is a binary classification model that classifies pancreatic cancer, and the component algorithms of the ensemble include tree-based models (Extra-trees (ET), Light-GBM (LGBM), random-forest (RF), gradient-boosting (GB), XGBoost (XGB), and Ada-boost (Ada) algorithms) and neural network-based multi-layer perceptron (MLP). Default values are used for hyperparameters of individual algorithms, and the MLP model may consist of one hidden layer consisting of a total of 16 neurons. The final prediction result may be evaluated by combining the prediction results of individual models using a logistic regression algorithm. However, the present invention is not limited to above description, and may be any type of machine learning model that can diagnose pancreatic cancer using the data obtained by measuring the expression levels of the biomarkers of the present invention.

According to a specific embodiment of the present invention, the biological sample is blood.

In the present invention, the input value of the machine learning model may be a measured value for the expression level of the polypeptide in “blood,” a biological sample, without being limited thereto. Here, the “biological sample” refers to any material, biological fluid, tissue or cells obtained or derived from the subject. For example, the biological sample may include whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid. Preferably, the biological sample may be a liquid biopsy (e.g., patient's tissue, cells, blood, serum, plasma, saliva, sputum or ascites, etc.) collected for histopathological examination by inserting a hollow needle or the like into an in vivo organ without incision of the skin of a patient having a high likelihood of developing pancreatic cancer.

According to a specific embodiment of the present invention, the measured value for the expression level of the polypeptide is a quantitative value obtained by mass spectrometry.

According to a specific embodiment of the present invention, the mass spectrometry is liquid chromatography-tandem mass spectrometry (LC-MS/MS).

According to a specific embodiment of the present invention, the pancreatic cancer is pancreatic ductal adenocarcinoma (PDAC).

In the present invention, mass spectrometry for measuring the expression level of the polypeptide and pancreatic ductal adenocarcinoma have already been described in detail, and thus detailed description thereof will be omitted below to avoid excessive overlapping.

### Advantageous Effects

The features and advantages of the present invention are summarized as follows:
(a) The present invention provides a method capable of predicting the onset of pancreatic cancer with high accuracy by measuring the expression levels of genes or proteins involved in the onset of pancreatic cancer.
(b) The present invention discovers effective biomarkers for pancreatic cancer, especially pancreatic ductal adenocarcinoma, and thus provides a multifaceted, comprehensive and novel therapeutic strategy for early diagnosis of pancreatic cancer, including a method capable of predicting the onset of pancreatic cancer at an early stage with high reliability. Ultimately, the present invention may be advantageously used to improve the survival rate against pancreatic cancer.

### BRIEF DESCRIPTION OF DRAWINGS

FIG. 1 shows chromatograms for 84 types of peptides (5 μL of 10 pg/μL in 0.1% formic acid, respectively).

FIG. 2 depicts graphs showing the results of analyzing the sensitivity distribution and analysis stability obtained in the process of optimizing the column oven temperature.

FIG. 3 depicts graphs showing the results of analyzing the maximum sensitivity distribution and analysis stability under each flow rate condition.

FIG. 4 shows chromatograms according to the existing concentration gradient (top) and the concentration gradient after change following optimization (bottom), respectively.

FIG. 5 is a graph showing the sensitivity distribution depending on GS1 condition.

FIG. 6 is a graph showing the sensitivity distribution depending on GS2 condition.

FIG. 7 is a graph showing the sensitivity distribution depending on TEM (Ion Source Temperature) condition.

FIG. 8 is a graph showing the sensitivity distribution depending on IS condition.

FIG. 9 is a graph showing the sensitivity distribution depending on CUR condition.

FIG. 10 is a graph showing the sensitivity distribution depending on CUR condition.

FIG. 11 shows an example of MRM and MS2 analysis for a light peptide standard.

FIG. 12 shows an example of optimization (CXP) of conditions for a target peptide.

FIG. 13 shows an example of a standard spiking test for detection and confirmation of endogenous peptide.

FIG. 14 shows detection of each candidate marker depending on the protein concentration in blood.

FIG. 15 shows an example of the results of a heavy interference test for isotope determination.

FIG. 16 shows an example of the results of a heavy interference test for isotope determination.

FIG. 17 shows the structure of a prediction model (stacking ensemble).

FIG. 18 shows the performance results (ROC curve) of the prediction model (stacking ensemble).

### MODE FOR INVENTION

Hereinafter, the present invention will be described in more detail by way of examples. These examples are only for illustrating the present invention in more detail, and it will be apparent to those skilled in the art that the scope of the present invention according to the subject matter of the present invention is not limited by these examples.

### EXAMPLES

### Experimental Methods

### 1. Marker Discovery

### Sample Collection Method

For the present invention, studies were conducted on a total of 80 serum samples collected from 2015 to 2021, among residual serum samples from normal people and pancreatic cancer patients stored in the Biobank of Seoul National University Hospital. The normal samples used in the present invention were 40 samples collected from people who were confirmed to have no pancreatic cancer through a standard examination at the time of blood collection and who had no history of diagnosis of other cancers, including pancreatic cancer, within the past 10 years from the time of blood collection. Pancreatic cancer samples were samples from 40 patients with pathologically confirmed pancreatic cancer (pancreatic ductal adenocarcinoma type), collected before surgery. The samples were collected from 35- to 81-year-old adults over 20 years old, and the distribution of pancreatic cancer samples by stage was confirmed to be 9 cases with AJCC stage 1, 6 cases with AJCC stage 2, 15 cases with AJCC stage 3, and 10 cases with AJCC stage 4. Samples collected in the study “Bio-Repository Construction for Liver, Biliary Tract, Pancreas, and Tumor Research (Seoul National University Hospital IRB No. 0901-010-267)” were preferentially used, and other samples were obtained from the Biobank of Seoul National University Hospital and used.

### Profiling Analysis

### Blood Sample Preprocessing

Serum samples were used without depletion of highly abundant proteins (albumin, transferrin, immunoglobulin, etc.). 5 μL of serum was added to a buffer containing urea (final 8 M) and dithiothreitol (DTT, final 19 mM) reagent and incubated at 37° C. for 1 hour and 30 minutes. After the incubation, the solution was cooled to room temperature and incubated with iodoacetamide (IAA, final 26 mM) for 30 minutes at room temperature in the dark. For subsequent trypsin digestion, the solution was diluted to a urea concentration of less than 1M with ammonium bicarbonate (final 100 mM) buffer. 5 ug (about 1:50 w/w) of trypsin was added thereto, followed by incubation at 37° C. for 16 hours. Thereafter, trifluoroacetic acid (TFA, final 1% TFA) was added to terminate the trypsin reaction. Then, desalting was performed using a solid-phase extraction (SPE) HRP plate. The desalting process involved activating the column with 800 μl of a solution containing 0.1% trifluoroacetic acid (TFA) and 80% acetonitrile (ACN), followed by an equilibration step with 800 μl of 0.1% trifluoroacetic acid (TFA). Next, the digested peptide was loaded onto the column. The column was washed three times with 800 μl of 0.1% trifluoroacetic acid (TFA) each, and then eluted using 300 μl of a solution containing 0.1% trifluoroacetic acid (TFA) and 80% acetonitrile (ACN). The completely dried samples were stored at −80° C. until mass spectrometry. In summary, the desalting process consists sequentially of column activation, equilibration, loading of the digested peptide sample, washing, peptide elution, and solvent drying. Thereafter, the dried samples were reconstituted in 0.1% trifluoroacetic acid (TFA), and the concentrations were measured at a wavelength of 280 nm using a Nanodrop instrument. The concentrations of all the samples were adjusted to 0.2 μg/μl by referring to the measured concentration values.

### Data-Independent Mass Spectrometry

Data-independent mass spectrometry (DIA) was performed using DIonex Ultimate 3000 RSLCnano/DO-RPLC reversed-phase liquid chromatography and an Orbitrap Exploris™ 480 mass spectrometer system. In the LC system, the sample was loaded into the trap column (0.1 mm×20 mm, 5 μm) using 100% Solvent A (0.1% formic acid and 5% dimethyl sulfoxide in water). Then, in the analytical column (50 cm×75 μm, 2 μm), a 80-min gradient was performed from 15 to 40% Solvent B (0.1% formic acid and 5% DMSO in 80% acetonitrile) at 50° C., and separation was performed for a total of 120 minutes (0.3 μl/min).

The MS system was operated in positive mode, and the ion transfer tube temperature and the spray voltage were set to 275° C. and 2.5 kV, respectively. For DIA, full MS resolutions were set to 60,000, full MS AGC target was 500% with an IT of 25 ms, and the m/z range was set to 350-1,500. AGC target value for MS2 spectra was set to 300% with an IT of 22 ms. 9 Da ions were collected in the precursor mass range of 385 to 1,015 m/z to obtain the MS2 spectrum. In addition, the resolution was set to 15,000, the collision energy in the HCD cell was set to 28%, and other parameters were set to default values. Detailed conditions are shown in Table 1 below.

TABLE 1

| LC-MS/MS System | Thermo Scientific Ultimate 3000/Thermo | | |
|---|---|---|---|
| | Scientific Orbitrap Exploris ™ 480 | | |
| Trap Column | Acclaim PepMap 100 C18 HPLC | | |
| | Columns (0.1 mm × 20 mm, 5 μm) | | |
| Analytical Column | Easy-spray PepMap RSLC C18 | | |
| | (50 cm × 75 μm, 2 μm) | | |
| Column Temp. | 50° C. | | |
| Flow rate | 0.3 μl/min | | |
| Ion source type | ESI positive-ion mode | | |
| Positive ion | 2,500 V | | |
| Spray voltage | | | |
| Negative ion | 600 V | | |
| Spray voltage | | | |
| Ion Transfer | 275° C. | | |
| tube Temp. | | | |
| Injection volume | 5 μl | | |
| Injection amount | 1 μg | | |
| Mobile phase | A: 0.1% formic acid and | | |
| | 5% DMSO in water, | | |
| | B: 0.1% formic acid and | | |
| | 5% DMSO in 80% acetonitrile | | |
| | | Solvent | Solvent |
| | Time | A (%) | B (%) |
| Gradient | 0 | 95 | 5 |
| | 5 | 95 | 15 |
| | 85 | 65 | 40 |
| | 90 | | 95 |
| | 100 | | 95 |
| | 105 | 95 | 5 |
| | 120 | 95 | 5 |

MS1 parameter - Full scan

| | |
|---|---|
| Orbitrap Resolution | 60,000 |
| Scan Range | 350-1,500 m/z |
| Automatic gain control | 500% (5 × 106) |
| (AGC) Target | |
| Maximum Injection | 20 ms |
| Time (IT) | |

MS2 parameter - DIA

| | |
|---|---|
| Orbitrap Resolution | 15,000 |
| Scan Range | 350-1,500 m/z |
| Precursor Mass Range | 385-1,015 m/z |
| Automatic gain control | 300% (3 × 105) |
| (AGC) Target | |
| Maximum Injection | 22 ms |
| Time (IT) | |
| HCD Collision Energy | 28% |
| (NCE) | |
| Isolation Window | 9 m/z |

### Marker Candidate Selection

### Data Processing

The LC-DIA-MS/MS data were processed using DIA-NN (version 1.8) with the in-house serum spectral library. Spectra were searched with default settings, except that MBR (Match-between-runs) was activated. Identification results were filtered at FDR (false discovery rate) of 1% at precursor and protein level. Peptide and protein quantities were obtained using the MaxLFQ algorithm, and the quantity data were subjected to quantile normalization.

### Differential Expression Analysis

An integrative statistical method was applied to define differentially expressed proteins (DEP). In brief, the present inventors calculated test statistics for each protein using Students' t-test, Wilcoxon-Ranksum test, and log 2-median-ratio in each comparison. Then, empirical distributions of the test statistics and log 2 median ratios for the null hypothesis were estimated by randomly permutating all samples 10,000 times. Using the estimated empirical distributions for each protein, the present inventors computed adjusted p-values for the observed test statistics and log 2 median ratio, and then calculated the overall p-value by combining these p-values using Stouffer's method. Finally, the present inventors identified DEPs as those with overall p-values <0.05 and absolute log 2 median ratios greater than the 5^{th} and 95^{th} percentile means of the empirical distribution in each comparison. Only proteins expressed in more than 25% of the total samples of each of the two test groups in each comparison were used for hypothesis testing.

### Target Peptide Selection

For selection of target peptides, an in silico peptide library was generated for each protein candidate using the UniProtKB/SwissProt human protein database. Target peptide candidates were limited to peptides that met the following criteria: unique in the human database; fully tryptic peptides; having no missed cleavage; consisting of 7 to 25 amino acids; not containing cysteine and methionine; and not corresponding to the N-terminal sequence of the protein. Additionally, a checklist was created to evaluate various properties of each peptide. For each peptide, the number of ragged tryptic sites, the number of asparagine/glutamine/proline amino acids, the presence or absence of an N-glycosylation motif, and the presence or absence of N-terminal glutamine were considered. From this peptide list, three target peptides for each protein candidate were selected. For this purpose, peptides included in public MRM databases such as CPTAC assay and SRMAtlas were first selected. In addition, the Human Plasma Peptide Atlas database was used to search whether peptides had previously been identified in the human plasma proteome. When the target peptides were insufficient, additional peptides were selected based on the presence or absence of a ragged tryptic site, the presence or absence of an N-glycosylation motif and N-terminal glutamine, and the number of asparagine/glutamine/proline amino acids.

### 2. Development of Analytical Method

### Establishment of Analysis Conditions

### Setting of Analysis Conditions

The present inventors sought to establish the conditions to be commonly applied to various biomarkers to be included in PANCCHECK, that is, optimal conditions for flow rate and column oven temperature in liquid chromatography (LC), and GS1 (nebulizer gas), GS2 (heating gas), TEM (ion source temperature), IS (ion spray voltage), CUR (curtain gas), and CAD (collision gas) in MS parameters.

### Peptide Selection

In order to optimize the above conditions, the present inventors sought to select peptides to be used in setting of the analysis conditions among 84 existing peptide standards that vary in length, hydrophobicity, and charge (Table 2).

TABLE 2
List of 84 existing peptide standards

| No. | Gene | Sequence |
|---|---|---|
| 1 | ALDOA | ADDGRPFPQVIK (SEQ ID NO: 16) |
| 2 | ALDOA | AAQEEYVK (SEQ ID NO: 17) |
| 3 | ANPEP | AEFNITLIHPK (SEQ ID NO: 18) |
| 4 | ANPEP | NATLVNEADK (SEQ ID NO: 19) |
| 5 | C5 | FQNSAILTIQPK (SEQ ID NO: 20) |
| 6 | C5 | VFQFLEK (SEQ ID NO: 21) |
| 7 | C7 | LIDQYGTHYLQSGSLGGEYR (SEQ ID NO: 22) |
| 8 | C7 | LTPLYELVK (SEQ ID NO: 23) |
| 9 | CPB1 | AEDTVTVENVLK (SEQ ID NO: 24) |
| 10 | CPB1 | ELASLHGTK (SEQ ID NO: 25) |
| 11 | DOCK10 | FVFETPFTLSGK (SEQ ID NO: 26) |
| 12 | DOCK10 | LTGLSEISQR (SEQ ID NO: 27) |
| 13 | EVC2 | TSEGFQAFSK (SEQ ID NO: 28) |
| 14 | EVC2 | TVEDAGQYLHQK (SEQ ID NO: 29) |
| 15 | FERMT3 | TASGDYIDSSWELR (SEQ ID NO: 30) |
| 16 | FERMT3 | VVLAGGVAPALFR (SEQ ID NO: 31) |
| 17 | FGA | GSESGIFTNTK (SEQ ID NO: 32) |
| 18 | FGA | ESSSHHPGIAEFPSR (SEQ ID NO: 33) |
| 19 | FGB | QGFGNVATNTDGK (SEQ ID NO: 34) |
| 20 | FGB | AHYGGFTVQNEANK (SEQ ID NO: 35) |
| 21 | FGG | IHLISTQSAIPYALR (SEQ ID NO: 36) |
| 22 | FGG | YEASILTHDSSIR (SEQ ID NO: 37) |
| 23 | GAPDH | VGVNGFGR (SEQ ID NO: 38) |
| 24 | GAPDH | GALQNIIPASTGAAK (SEQ ID NO: 39) |
| 25 | GP5 | LPNLSSLTLSR (SEQ ID NO: 40) |
| 26 | GP5 | YLGVTLSPR (SEQ ID NO: 41) |
| 27 | HP | VGYVSGWGR (SEQ ID NO: 42) |
| 28 | HP | HYEGSTVPEK (SEQ ID NO: 43) |
| 29 | HSPA2 | DAGTITGLNVLR (SEQ ID NO: 44) |
| 30 | HSPA2 | EIAEAYLGGK (SEQ ID NO: 45) |
| 31 | ICAM1 | LLGIETPLPK (SEQ ID NO: 46) |
| 32 | ICAM1 | VTLNGVPAQPLGPR (SEQ ID NO: 47) |
| 33 | IGFBP2 | HGLYNLK (SEQ ID NO: 48) |
| 34 | IGFBP2 | LIQGAPTIR (SEQ ID NO: 7) |
| 35 | ITGA2B | ALSNVEGFER (SEQ ID NO: 49) |
| 36 | ITGA2B | VAIVVGAPR (SEQ ID NO: 50) |
| 37 | ITIH3 | SLPEGVANGIEVYSTK (SEQ ID NO: 51) |
| 38 | ITIH3 | EVSFDVELPK (SEQ ID NO: 52) |
| 39 | KLKB1 | IYSGILNLSDITK (SEQ ID NO: 53) |
| 40 | KLKB1 | IAYGTQGSSGYSLR (SEQ ID NO: 54) |
| 41 | LBP | ITLPDFTGDLR (SEQ ID NO: 55) |
| 42 | LBP | LAEGFPLPLLK (SEQ ID NO: 56) |
| 43 | LDHB | LIAPVAEEEATVPNNK (SEQ ID NO: 57) |
| 44 | LDHB | DDEVAQLK (SEQ ID NO: 58) |
| 45 | LRG1 | LPPGLLANFTLLR (SEQ ID NO: 59) |
| 46 | LRG1 | DLLLPQPDLR (SEQ ID NO: 60) |
| 47 | MBL2 | EEAFLGITDEK (SEQ ID NO: 61) |
| 48 | MBL2 | WLTFSLGK (SEQ ID NO: 62) |
| 49 | MMP9 | AFALWSAVTPLTFTR (SEQ ID NO: 63) |
| 50 | MMP9 | AVIDDAFAR (SEQ ID NO: 64) |
| 51 | MRC1 | TGIAGGLWDVLK (SEQ ID NO: 65) |
| 52 | MRC1 | LITASGSYHK (SEQ ID NO: 66) |
| 53 | PDIA6 | TGEAIVDAALSALR (SEQ ID NO: 67) |
| 54 | PDIA6 | GSTAPVGGGAFPTIVER (SEQ ID NO: 68) |
| 55 | PIGR | LVSLTLNLVTR (SEQ ID NO: 69) |
| 56 | PIGR | VPGNVTAVLGETLK (SEQ ID NO: 70) |
| 57 | PPBP | EESLDSDLYAELR (SEQ ID NO: 71) |
| 58 | PPBP | TTSGIHPK (SEQ ID NO: 72) |
| 59 | PPIA | VSFELFADK (SEQ ID NO: 73) |
| 60 | PPIA | FEDENFILK (SEQ ID NO: 74) |
| 61 | PRDX2 | LSEDYGVLK (SEQ ID NO: 75) |
| 62 | PRDX2 | ATAVVDGAFK (SEQ ID NO: 76) |
| 63 | PRDX6 | LIALSIDSVEDHLAWSK (SEQ ID NO: 77) |
| 64 | PRDX6 | LPFPIIDDR (SEQ ID NO: 78) |
| 65 | RAB10 | AFLTLAEDILR (SEQ ID NO: 79) |
| 66 | RAB10 | FHTITTSYYR (SEQ ID NO: 80) |
| 67 | S100A8 | ALNSIIDVYHK (SEQ ID NO: 81) |
| 68 | S100A8 | GADVWFK (SEQ ID NO: 82) |
| 69 | SAA1 | GPGGVWAAEAISDAR (SEQ ID NO: 83) |
| 70 | SAA1 | FFGHGAEDSLADQAANEWGR (SEQ ID NO: 84) |
| 71 | SERPINA5 | AVVEVDESGTR (SEQ ID NO: 85) |
| 72 | SERPINA5 | EDQYHYLLDR (SEQ ID NO: 86) |
| 73 | TIMP1 | FVGTPEVNQTTLYQR (SEQ ID NO: 87) |
| 74 | TIMP1 | GFQALGDAADIR (SEQ ID NO: 88) |
| 75 | TPM4 | IQALQQQADEAEDR (SEQ ID NO: 89) |
| 76 | TPM4 | ASDAEGDVAALNR (SEQ ID NO: 90) |
| 77 | VCL | STVEGIQASVK (SEQ ID NO: 91) |
| 78 | VCL | IPTISTQLK (SEQ ID NO: 92) |
| 79 | VWF | DGTVTTDWK (SEQ ID NO: 93) |
| 80 | VWF | HIVTFDGQNFK (SEQ ID NO: 94) |
| 81 | YWHAH | AVTELNEPLSNEDR (SEQ ID NO: 95) |
| 82 | YWHAH | NSVVEASEAAYK (SEQ ID NO: 96) |
| 83 | YWHAZ | SVTEQGAELSNEER (SEQ ID NO: 97) |
| 84 | YWHAZ | FLIPNASQAESK (SEQ ID NO: 98) |

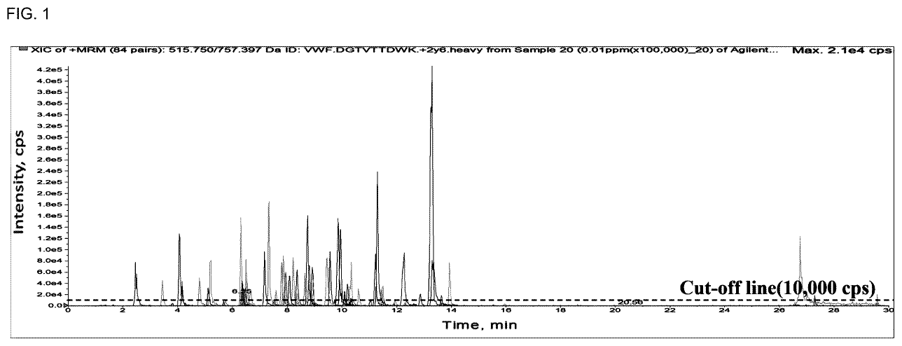

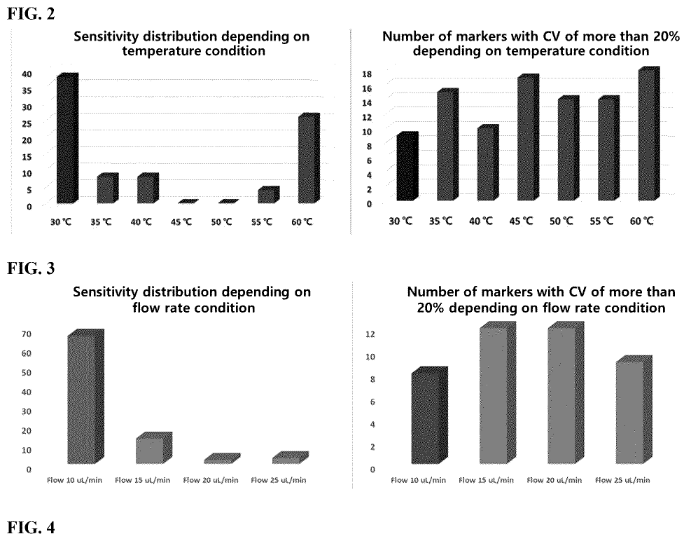

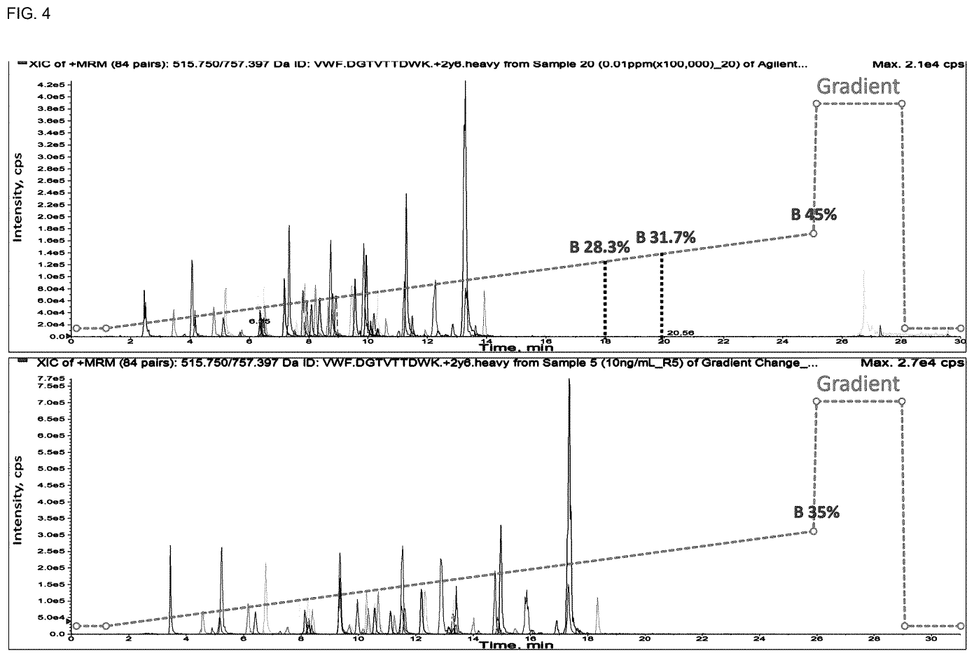

In general, 10,000 cps (counts per second) is the level of intensity that is capable of providing reproducible analysis results when 50 μg of each standard is injected and analyzed in LC-MS/MS (liquid chromatography-tandem mass spectrometry) under existing analysis conditions (Table 3). 55 peptides with an intensity higher than 10,000 cps were selected as sample peptides for setting analysis conditions (Table 4 and FIG. 1).

TABLE 3
Existing analysis conditions

| | |
|---|---|
| LC & LC-MS/MS | SCIEX M5 micro LC & |
| system | SCIEX QTRAP 5500+ |
| Column | Agilent ZORBAX |
| | 300SB-C18(0.5 × 150 mm, 3.5 um) |
| Column oven | 35° C. |
| Temp. | |
| Flow rate | 20 μL/min |
| Ionization | ESI positive-ion mode |
| Curtain gas | 10 psi |
| Collision gas | 8 psi |
| Ion spray voltage | 5,500 |
| Ion source Temp. | 500° C. |
| Ion source gas 1 | 15 psi |
| (GS1) | |
| Ion source gas 2 | 50 psi |
| (GS2) | |
| Injection volume | 5 μL |
| Mobile phase | A: 0.1% formic acid in water, |
| | B: 0.1% formic acid in acetonitrile |

| | Time | Solvent | Solvent |
|---|---|---|---|
| | (min) | A (%) | B (%) |
| Gradient | 0 | 95 | 5 |
| | 1 | 95 | 5 |
| | 25 | 55 | 45 |
| | 25.1 | 0 | 100 |
| | 28 | 0 | 100 |
| | 28.1 | 95 | 5 |
| | 30 | 95 | 5 |

TABLE 4

| No. | Gene | Sequence | Intensity (cps) |
|---|---|---|---|
| 1 | ALDOA | AAQEEYVK | 45,436 |
| 2 | ANPEP | AEFNITLIHPK (SEQ ID | 35,122 |
| | | NO: 18) | |
| 3 | ANPEP | NATLVNEADK (SEQ ID | 49,543 |
| | | NO: 19) | |
| 4 | C5 | FQNSAILTIQPK (SEQ ID | 13,803 |
| | | NO: 20) | |
| 5 | C5 | VFQFLEK (SEQ ID NO: 21) | 238,595 |
| 6 | C7 | LTPLYELVK (SEQ ID NO: | 426,875 |
| | | 23) | |
| 7 | CPB1 | AEDTVTVENVLK (SEQ ID | 26,112 |
| | | NO: 24) | |
| 8 | CPB1 | ELASLHGTK (SEQ ID NO: | 128,389 |
| | | 25) | |
| 9 | DOCK10 | LTGLSEISQR (SEQ ID NO: | 86,230 |
| | | 27) | |
| 10 | EVC2 | TSEGFQAFSK (SEQ ID | 60,464 |
| | | NO: 28) | |
| 11 | EVC2 | TVEDAGQYLHQK (SEQ | 156,931 |
| | | ID NO: 29) | |
| 12 | FERMT3 | VVLAGGVAPALFR (SEQ | 18,906 |
| | | ID NO: 31) | |
| 13 | FGA | GSESGIFTNTK (SEQ ID | 28,014 |
| | | NO: 32) | |
| 14 | FGA | ESSSHHPGIAEFPSR (SEQ | 83,248 |
| | | ID NO: 33) | |
| 15 | FGB | AHYGGFTVQNEANK | 44,034 |
| | | (SEQ ID NO: 35) | |
| 16 | FGG | YEASILTHDSSIR (SEQ ID | 22,008 |
| | | NO: 37) | |
| 17 | GAPDH | VGVNGFGR (SEQ ID NO: | 24,611 |
| | | 38) | |
| 18 | GAPDH | GALQNIIPASTGAAK | 78,207 |
| | | (SEQ ID NO: 39) | |
| 19 | GP5 | YLGVTLSPR (SEQ ID NO: | 156,127 |
| | | 41) | |
| 20 | HP | VGYVSGWGR (SEQ ID | 64,473 |
| | | NO: 42) | |
| 21 | HP | HYEGSTVPEK (SEQ ID | 78,007 |
| | | NO: 43) | |
| 22 | HSPA2 | DAGTITGLNVLR (SEQ ID | 28,714 |
| | | NO: 44) | |
| 23 | HSPA2 | EIAEAYLGGK (SEQ ID | 89,240 |
| | | NO: 45) | |
| 24 | ICAM1 | LLGIETPLPK (SEQ ID NO: | 94,757 |
| | | 46) | |
| 25 | ICAM1 | VTLNGVPAQPLGPR (SEQ | 96,664 |
| | | ID NO: 47) | |
| 26 | IGFBP2 | HGLYNLK (SEQ ID NO: | 11,502 |
| | | 48) | |
| 27 | IGFBP2 | LIQGAPTIR (SEQ ID NO: | 186,005 |
| | | 7) | |
| 28 | ITGA2B | ALSNVEGFER (SEQ ID | 58,660 |
| | | NO: 49) | |
| 29 | ITGA2B | VAIVVGAPR (SEQ ID NO: | 77,204 |
| | | 50) | |
| 30 | ITIH3 | EVSFDVELPK (SEQ ID | 11,902 |
| | | NO: 52) | |
| 31 | KLKB1 | IAYGTQGSSGYSLR (SEQ | 11,302 |
| | | ID NO: 54) | |
| 32 | LDHB | LIAPVAEEEATVPNNK | 55,254 |
| | | (SEQ ID NO: 57) | |
| 33 | LDHB | DDEVAQLK (SEQ ID NO: | 81,015 |
| | | 58) | |
| 34 | MBL2 | EEAFLGITDEK (SEQ ID | 30,116 |
| | | NO: 61) | |
| 35 | MBL2 | WLTFSLGK (SEQ ID NO: | 77,806 |
| | | 62) | |
| 36 | MMP9 | AVIDDAFAR (SEQ ID NO: | 161,155 |
| | | 64) | |
| 37 | MRC1 | LITASGSYHK (SEQ ID | 33,820 |
| | | NO: 66) | |
| 38 | PPIA | VSFELFADK (SEQ ID NO: | 21,308 |
| | | 73) | |
| 39 | PPIA | FEDENFILK (SEQ ID NO: | 92,450 |
| | | 74) | |
| 40 | PRDX2 | LSEDYGVLK (SEQ ID NO: | 53,450 |
| | | 75) | |
| 41 | PRDX2 | ATAVVDGAFK (SEQ ID | 96,864 |
| | | NO: 76) | |
| 42 | PRDX6 | LPFPIIDDR (SEQ ID NO: | 81,115 |
| | | 78) | |
| 43 | RAB10 | FHTITTSYYR (SEQ ID NO: | 27,914 |
| | | 80) | |
| 44 | S100A8 | ALNSIIDVYHK (SEQ ID | 12,703 |
| | | NO: 81) | |
| 45 | S100A8 | GADVWFK (SEQ ID NO: | 136,124 |
| | | 82) | |
| 46 | SERPINA5 | AVVEVDESGTR (SEQ ID | 32,218 |
| | | NO: 85) | |
| 47 | SERPINA5 | EDQYHYLLDR (SEQ ID | 72,091 |
| | | NO: 86) | |
| 48 | TIMP1 | GFQALGDAADIR (SEQ ID | 38,626 |
| | | NO: 88) | |
| 49 | TPM4 | ASDAEGDVAALNR (SEQ | 31,117 |
| | | ID NO: 90) | |
| 50 | VCL | STVEGIQASVK (SEQ ID | 31,317 |
| | | NO: 91) | |
| 51 | VCL | IPTISTQLK (SEQ ID NO: | 68,783 |
| | | 92) | |
| 52 | VWF | DGTVTTDWK (SEQ ID | 20,607 |
| | | NO: 93) | |
| 53 | VWF | HIVTFDGQNFK (SEQ ID | 58,360 |
| | | NO: 94) | |
| 54 | YWHAH | NSVVEASEAAYK (SEQ ID | 46,438 |
| | | NO: 96) | |
| 55 | YWHAZ | FLIPNASQAESK (SEQ ID | 85,528 |
| | | NO: 98) | |

### Optimization of Column Oven Temperature

Column oven temperature is a factor that comprehensively affects peak resolution, peak shape, column life, sensitivity, and the like. Agilent ZORBAX 300SB-C18 (0.5×150 mm, 3.5 μm) was used as the analytical column, and the column oven temperature was examined up to 60° C. In general, tailing in the peak of the peptide may occur at low temperatures, and there is a possibility that variability may occur due to external temperature depending on the season, etc., and for this reason, temperatures below 30° C. were not examined.

### Optimization of Flow Rate

A total of four flow rate conditions were examined from 10 μL/min to 25 μL/min at intervals of 5 μL/min, considering column pressure and settable conditions. In the case of M5 microLC, a minimum of 5 μL/min can be set, but it was excluded from the examination because the rate and flow of the mobile phase are highly variable under this condition.

When a total of four conditions were compared and analyzed from 10 μL/min to 25 μL/min at intervals of 5 μL/min, the largest number of peptides showed the highest sensitivity at 10 μL/min. In addition, it was confirmed that, in the case of coefficient of variation (CV %) obtained through five repeated analysis, the number of peptides exceeding 20 CV % was the lowest at 10 μL/min, suggesting that precision was also the best at 10 μL/min. Thus, the flow rate for the M5 microLC in use was set at 10 μL/min.

### Concentration Gradient Change

The present inventors sought to change the concentration gradient in order to improve the separation of peaks according to multiple peptide analysis while being suitable for the changed flow rate. The existing condition that increases the proportion of solvent B, which is an organic solvent, among mobile phase solvents, to 45%, was changed to the condition that increases the proportion of solvent B to 35%, so that the elution of peptides from the column was lowered to improve the separation of each peak. As a result, it could be confirmed that the peptide, which was last eluted at a retention time (Rt.) of 14 minutes under the existing conditions, was eluted at a delayed time of 18.5 minutes after the gradient change, so that the separation of each peak was improved. Although there is room to further improve separation by applying conditions that increase solvent B (organic solvent B) to a level lower than 35%, the concentration gradient condition that finally increases solvent B to 35% was applied, considering the elution of the matrix other than the target peptide, the possibility of adding peptides with high hydrophobicity in the future, and the occurrence of peak broadening phenomenon (Table 5).

TABLE 5

| Existing gradient | | | | | | | |
|---|---|---|---|---|---|---|---|
| | Sol- | Sol- | | Changed gradient | | | |
| | vent | vent | Flow | | | | Flow |
| Time | A | B | rate | Time | Solvent | Solvent | rate |
| (min) | (%) | (%) | (μL/min) | (min) | A (%) | B (%) | (μL/min) |
| 0 | 95 | 5 | 20 | 0 | 95 | 5 | 10 |
| 1 | 95 | 5 | | 1 | 95 | 5 | |
| 25 | 55 | 45 | | 25 | 65 | 35 | |
| 25.1 | 0 | 100 | | 25.1 | 0 | 100 | |
| 28 | 0 | 100 | | 28 | 0 | 100 | |
| 28.1 | 95 | 5 | | 28.1 | 95 | 5 | |
| 30 | 95 | 5 | | 31 | 95 | 5 | |

### Optimization of GS1 (Nebulizer Gas)

Nebulizer gas GS1 is a gas that helps form droplets when the target peptides separated through the column are sprayed through a nebulizer. For the nebulizer gas GS1, generally, the higher the flow rate, the higher the pressure is used. Although a pressure from 0 psi (the lowest setting condition of the Qtrap 5500+system) to 90 psi (the highest setting condition) can be examined, a pressure of up to 70 psi was examined because the high pressure of GS1 causes a stop phenomenon due to an increase in pressure inside the source.

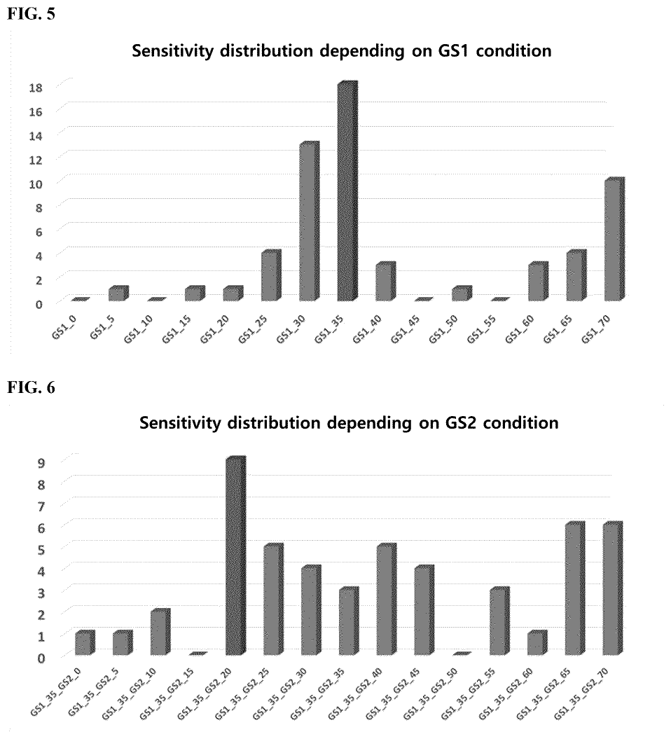

As a result of comparatively analyzing a total of 15 conditions from GS1 0 psi to 70 psi at intervals of 5 psi, it was confirmed that the distribution of peptides showing maximum sensitivity was the highest at 35 psi. Thus, the nebulizer gas GS1 was set to 35 psi (FIG. 5).

### Optimization of GS2 (Heating Gas)

GS2, a heating gas heated in the source and sprayed therefrom, serves to promote evaporation of droplets containing ions and ultimately condenses the charged analyte. Although the pressure range of GS2 can be examined from 0 psi (the lowest setting condition of the Qtrap 5500+system) to 90 psi (the highest setting condition), a pressure of up to 70 psi was examined because GS2 at high pressure causes a stop phenomenon due to an increase in pressure inside the source.

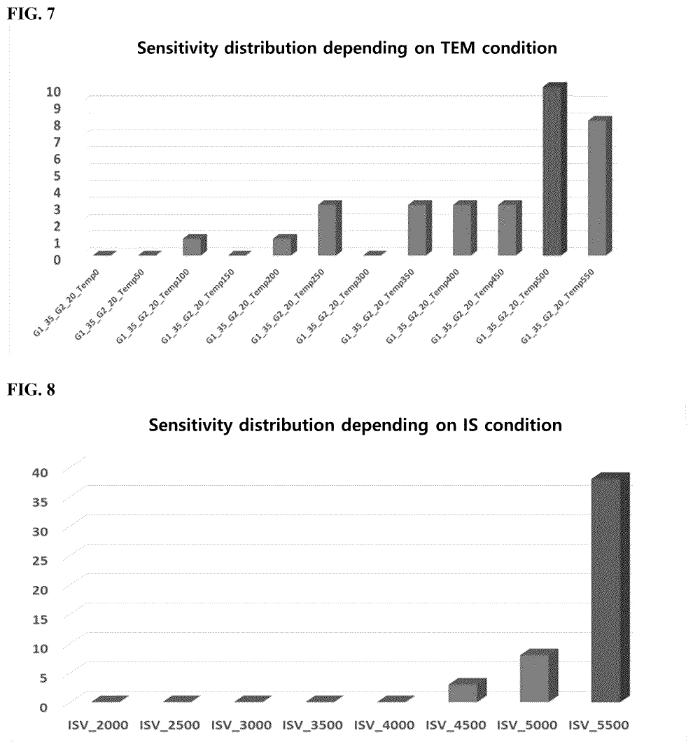

As a result of comparatively analyzing a total of 15 GS2 conditions from 0 psi to 70 psi at intervals of 5 psi, it was confirmed that the distribution of peptides showing maximum sensitivity was the highest at 20 psi. Thus, the heating gas GS2 was set to 20 psi (FIG. 6).

### Optimization of TEM (Ion Source Temperature)

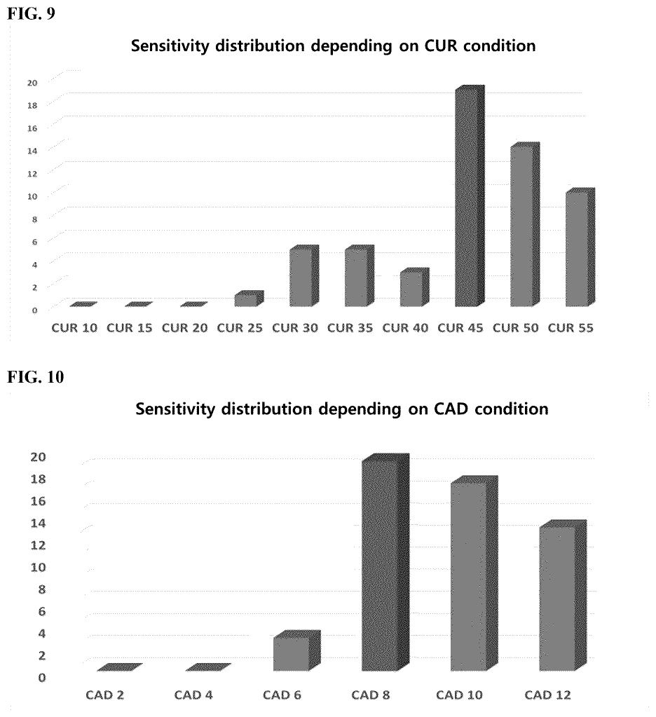

TEM plays a role in heating the heating gas GS2. Although TEM can be set up to 750° C., it was examined from 0° C. to a maximum of 550° C. because the electrode, which is adjacent to the source and can be affected by temperature, is partially made of a plastic material that is meltable. As a result of examining a total of 12 conditions from 0° C. to 550° C. at intervals of 50° C., the number of peptides showing maximum sensitivity tended to increase up to 500° C. and then decrease at 550° C. Therefore, 500° C., where the number of peptides showing maximum sensitivity was the largest, was set as the optimal TEM condition (FIG. 7).

### Optimization of IS (Ion Spray Voltage)

IS is a voltage applied between an electrode and an orifice plate (Corn) and is involved in introducing analyte ions into the MS. Although the Qtrap 5500+system used in-house can be examined up to 5,500V, a total of 8 IS conditions from 2,000V to 5,500V at intervals of 500V were examined because below 2,000V, most peptides are not detected or show very low sensitivity. Under the conditions examined, the sensitivity gradually increased as the voltage increased, and 5,500V, where the distribution of peptides showing maximum sensitivity was the highest, was set as the optimal IS condition (FIG. 8).

### Optimization of CUR (Curtain Gas)

CUR refers to nitrogen gas flowing between an orifice and a curtain plate, and serves to remove some remaining droplets and neutral ions, thus preventing contamination of the source and keeping Q0 clean. A total of 10 conditions from 10 psi (the lowest setting condition of the Qtrap 5500+system) to 55 psi (the highest setting condition) at intervals of 5 psi were examined. Among the conditions examined, 45 psi showed the highest distribution of peptides showing maximum sensitivity. Thus, the optimal CUR condition was set at 45 psi (FIG. 9).

### Optimization of CAD (Collision Gas)

Precursor ions are fragmented by collision with CAD to form productions. CAD was examined from 2 psi (the lowest setting condition) to 12 psi (the highest setting condition), and a total of six conditions at intervals of 2 psi were compared and analyzed. Among the conditions examined, 8 psi showed the highest distribution of peptides showing maximum sensitivity. Thus, the optimal CAD condition was set at 8 psi (FIG. 10).

### Optimization of Analysis Conditions

### MS2 Analysis and Selection of Top 3 Ions

### (1) Synthesis of Light Peptide Standards

To optimize individual analysis conditions for PANCCHECK candidate markers including 53 genes and 159 peptides selected through the above discovery process, light peptides identical to the endogenous peptides were used as standards for analysis. The light peptide standards were received and used after their sequences were sent to and synthesized by the peptide synthesis service team in the bio-production technology division of Bertis Inc., which operates a GMP (Good Manufacturing Practice) facility.

### (2) MS2 Analysis and Selection of Top 3 Ions

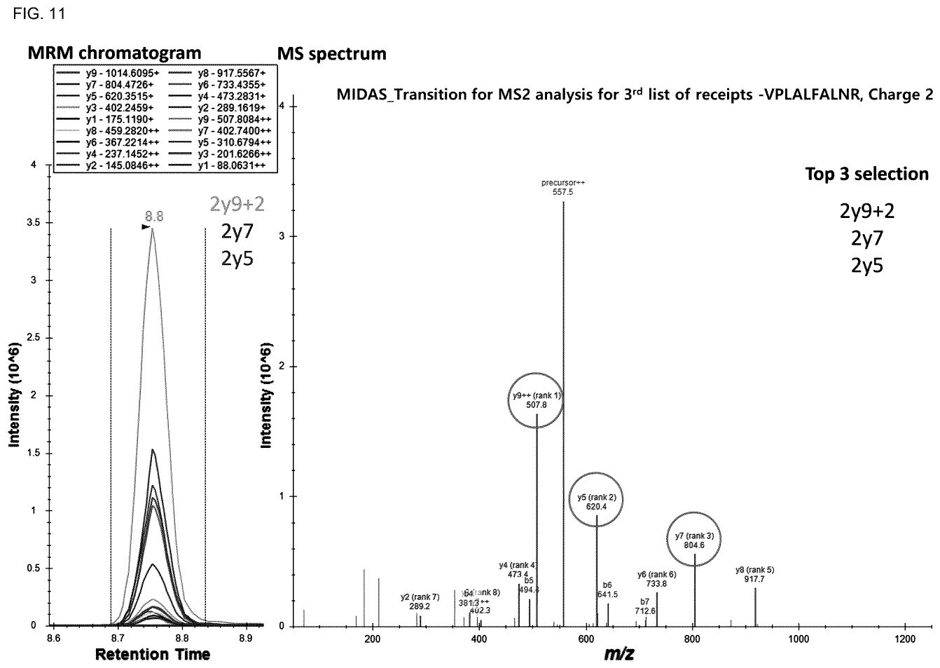

Through the MS spectra analyzed in the Qtrap 5500+system in EPI (enhanced product ion scan) mode, the equivalence between the received light peptide standard and the sent sequences was checked, and the MS spectra and MRM chromatograms were checked. Based on this, the top 3 ions to be used for MRM analysis were selected. The top 3 ions were selected by comprehensively examining the ions detected at high intensity in the MS spectra and the ions detected at high intensity in the MRM chromatograms (FIG. 11).

### Optimization of Target Peptide Conditions (Optimization of Individual Parameters)

yy

DP is a voltage applied to the orifice in order to minimize solvent clusters that may remain after analyte ions are introduced into the MS by vacuum. Although the settable DP range is 0 to 300, a total of 13 conditions in the range of 20 to 260 at intervals of 20 were examined for optimization of DP because an unnecessarily high DP value may cause the target peptide not to be detected and an excessively low DP value may cause orifice contamination as well as reduced sensitivity.

EP is a parameter involved in the passage of ions through Q0. A total of 7 EP conditions from 2 (the lowest setting condition) to 15 (the highest setting condition) at intervals of 2 were examined.

CE is a parameter directly involved in the fragmentation of precursor ions, and the precursor ions receive energy and are accelerated to a collision cell, where they collide with a collision gas (CAD) to form product ions. CE, can be set from a minimum of 5 to a maximum of 180 based on Qtrap5500+. Optimization of CE can be done by examining all CEs in the settable range, but the examination takes a lot of time. For this reason, a method of examining multiple points based on the value calculated by the skyline program is also applicable. In this experiment, a total of 10 conditions were examined, including 4 conditions downward and 5 conditions upward at intervals of 2 based on the CE value calculated by the Skyline program. In the case of markers whose sensitivity continued to rise or fall as the CE rose within the examined CE range, and for which the inflection point could not be confirmed, the examination section was extended and the optimal condition was set by reanalysis.

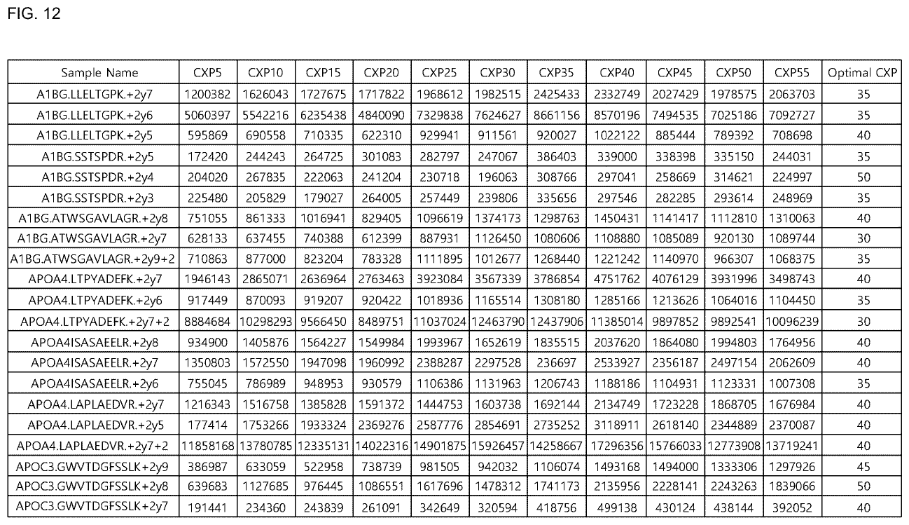

CXP is a parameter involved in the transfer of the product ions, formed by fragmentation in the collision cell, to Q3. An optimal CXP condition was set by examining a total of 11 conditions from 0 (the lowest setting condition) to 55 (the highest setting condition) at intervals of 5 (FIG. 12).

TABLE 6

| ID | DP | EP | CE | CXP |
|---|---|---|---|---|
| A1BG.LLELTGPK.+2y6.light | 100 | 5 | 22.4 | 35 |
| A1BG.LLELTGPK.+2y6.heavy | 100 | 5 | 22.4 | 35 |
| A1BG.SSTSPDR.+2y5.light | 60 | 5 | 15.4 | 35 |
| A1BG.SSTSPDR.+2y5.heavy | 60 | 5 | 15.4 | 35 |
| A1BG.ATWSGAVLAGR.+2y8.light | 100 | 5 | 31.7 | 40 |
| A1BG.ATWSGAVLAGR.+2y8.heavy | 100 | 5 | 31.7 | 40 |
| APOA4.LTPYADEFK.+2y7+2.light | 80 | 5 | 21.6 | 30 |
| APOA4.LTPYADEFK.+2y7+2.heavy | 80 | 5 | 21.6 | 30 |
| APOA4.ISASAEELR.+2y8.light | 100 | 5 | 28.9 | 40 |
| APOA4.ISASAEELR.+2y8.heavy | 100 | 5 | 28.9 | 40 |
| APOA4.LAPLAEDVR.+2y5.light | 100 | 5 | 27.1 | 40 |
| APOA4.LAPLAEDVR.+2y5.heavy | 100 | 5 | 27.1 | 40 |
| APOC3.GWVTDGFSSLK.+2y9.light | 100 | 9 | 26.3 | 45 |
| APOC3.GWVTDGFSSLK.+2y9.heavy | 100 | 9 | 26.3 | 45 |
| APOC3.DYWSTVK.+2y4.light | 60 | 2 | 25 | 40 |
| APOC3.DYWSTVK.+2y4.heavy | 60 | 2 | 25 | 40 |
| APOL1.NEADELR.+2y5.light | 80 | 5 | 23.8 | 45 |
| APOL1.NEADELR.+2y5.heavy | 80 | 5 | 23.8 | 45 |
| APOL1.ALADGVQK.+2y6.light | 80 | 2 | 18.7 | 35 |
| APOL1.ALADGVQK.+2y6.heavy | 80 | 2 | 18.7 | 35 |
| ALDOA.ELSDIAHR.+3y6+2.light | 60 | 5 | 15.1 | 45 |
| ALDOA.ELSDIAHR.+3y6+2.heavy | 60 | 5 | 15.1 | 45 |
| AMY2A.NWGEGWGFVPSDR.+2y4.light | 140 | 15 | 45.9 | 55 |
| AMY2A.NWGEGWGFVPSDR.+2y4.heavy | 140 | 15 | 45.9 | 55 |
| CD14.VDADADPR.+2y2.light | 40 | 5 | 26.1 | 15 |
| CD14.VDADADPR.+2y2.heavy | 40 | 5 | 26.1 | 15 |
| CD14.ELTLEDLK.+2y6+2.light | 60 | 2 | 20.6 | 25 |
| CD14.ELTLEDLK.+2y6+2.heavy | 60 | 2 | 20.6 | 25 |
| CD163.GADLSLR.+2y5.light | 80 | 5 | 14.9 | 45 |
| CD163.GADLSLR.+2y5.heavy | 80 | 5 | 14.9 | 45 |
| CRP.ESDTSYVSLK.+2y3.light | 120 | 5 | 24.7 | 25 |
| CRP.ESDTSYVSLK.+2y3.heavy | 120 | 5 | 24.7 | 25 |
| CRP.GYSIFSYATK.+2y6.light | 100 | 9 | 24.9 | 45 |
| CRP.GYSIFSYATK.+2y6.heavy | 100 | 9 | 24.9 | 45 |
| FCGR3A.AVVFLEPQWYR.+2y5.light | 120 | 15 | 39.5 | 50 |
| FCGR3A.AVVFLEPQWYR.+2y5.heavy | 120 | 15 | 39.5 | 50 |
| FCGR3A.YFHHNSDFYIPK.+4y2.light | 80 | 5 | 15.6 | 30 |
| FCGR3A.YFHHNSDFYIPK.+4y2.heavy | 80 | 5 | 15.6 | 30 |
| HGFAC.TTDVTQTFGIEK.+2y7.light | 140 | 9 | 29.8 | 40 |
| HGFAC.TTDVTQTFGIEK.+2y7.heavy | 140 | 9 | 29.8 | 40 |
| ITIH3.SLPEGVANGIEVYSTK.+2y14+2.light | 180 | 15 | 37.8 | 55 |
| ITIH3.SLPEGVANGIEVYSTK.+2y14+2.heavy | 180 | 15 | 37.8 | 55 |
| PFN1.DSPSVWAAVPGK.+2y10+2.light | 100 | 2 | 24.8 | 40 |
| PFN1.DSPSVWAAVPGK.+2y10+2.heavy | 100 | 2 | 24.8 | 40 |
| PFN1.STGGAPTFNVTVTK.+2y9.light | 140 | 5 | 30.8 | 45 |
| PFN1.STGGAPTFNVTVTK.+2y9.heavy | 140 | 5 | 30.8 | 45 |
| APOA1.DLATVYVDVLK.+2y6.light | 20 | 15 | 33.3 | 35 |
| APOA1.DLATVYVDVLK.+2y6.heavy | 20 | 15 | 33.3 | 35 |
| APOA1.AHVDALR.+2y4.light | 60 | 5 | 24.2 | 25 |
| APOA1.AHVDALR.+2y4.heavy | 60 | 5 | 24.2 | 25 |
| APOA1.ATEHLSTLSEK.+3y3.light | 80 | 2 | 21.5 | 40 |
| APOA1.ATEHLSTLSEK.+3y3.heavy | 80 | 2 | 21.5 | 40 |
| C9.VVEESELAR.+2y7.light | 80 | 2 | 22.3 | 45 |
| C9.VVEESELAR.+2y7.heavy | 80 | 2 | 22.3 | 45 |
| C9.GEIHLGR.+3y5+2.light | 80 | 2 | 12 | 40 |
| C9.GEIHLGR.+3y5+2.heavy | 80 | 2 | 12 | 40 |
| C9.ALPTTYEK.+2y6+2.light | 80 | 2 | 19.6 | 25 |
| C9.ALPTTYEK.+2y6+2.heavy | 80 | 2 | 19.6 | 25 |
| ALDOA.ALQASALK.+2y6.light | 60 | 2 | 18.7 | 40 |
| ALDOA.ALQASALK.+2y6.heavy | 60 | 2 | 18.7 | 40 |
| HP.DYAEVGR.+2y5.light | 60 | 2 | 22.9 | 35 |
| HP.DYAEVGR.+2y5.heavy | 60 | 2 | 22.9 | 35 |
| GP5.TLPAAAFR.+2y6+2.light | 80 | 9 | 17.8 | 25 |
| GP5.TLPAAAFR.+2y6+2.heavy | 80 | 9 | 17.8 | 25 |
| MCAM.VSPAAPER.+2y6+2.light | 60 | 2 | 17.3 | 25 |
| MCAM.VSPAAPER.+2y6+2.heavy | 60 | 2 | 17.3 | 25 |
| ICAM1.LLGIETPLPK.+2y8.light | 100 | 11 | 23.5 | 45 |
| ICAM1.LLGIETPLPK.+2y8.heavy | 100 | 11 | 23.5 | 45 |
| SERPINA3.ITLLSALVETR.+2y7.light | 120 | 15 | 36.8 | 55 |
| SERPINA3.ITLLSALVETR.+2y7.heavy | 120 | 15 | 36.8 | 55 |
| CNDP1.AIHLDLEEYR.+2y2.light | 160 | 13 | 35.9 | 35 |
| CNDP1.AIHLDLEEYR.+2y2.heavy | 160 | 13 | 35.9 | 35 |
| CNDP1.HLEDVFSK.+3b4.light | 80 | 2 | 11.6 | 30 |
| CNDP1.HLEDVFSK.+3b4.heavy | 80 | 2 | 11.6 | 30 |
| CNDP1.DGSTIPIAK.+2y4.light | 60 | 9 | 25.1 | 25 |
| CNDP1.DGSTIPIAK.+2y4.heavy | 60 | 9 | 25.1 | 25 |
| ITIH3.ALDLSLK.+2y5.light | 80 | 9 | 19.6 | 30 |
| ITIH3.ALDLSLK.+2y5.heavy | 80 | 9 | 19.6 | 30 |
| PEPD.AFTPFSGPK.+2y7.light | 80 | 9 | 18.3 | 35 |
| PEPD.AFTPFSGPK.+2y7.heavy | 80 | 9 | 18.3 | 35 |
| NOTCH2.ALGTLLHTNLR.+3y9+2.light | 80 | 9 | 15.4 | 45 |
| NOTCH2.ALGTLLHTNLR.+3y9+2.heavy | 80 | 9 | 15.4 | 45 |
| HGFAC.YEYLEGGDR.+2y7.light | 100 | 2 | 26 | 45 |
| HGFAC.YEYLEGGDR.+2y7.heavy | 100 | 2 | 26 | 45 |
| HGFAC.EALVPLVADHK.+3y7+2.light | 80 | 7 | 17.1 | 35 |
| HGFAC.EALVPLVADHK.+3y7+2.heavy | 80 | 7 | 17.1 | 35 |
| MASP1.IEPSQAK.+2y5.light | 60 | 2 | 15.9 | 35 |
| MASP1.IEPSQAK.+2y5.heavy | 60 | 2 | 15.9 | 35 |
| IGFBP3.FHPLHSK.+3y5+2.light | 60 | 2 | 15.9 | 35 |
| IGFBP3.FHPLHSK.+3y5+2.heavy | 60 | 2 | 15.9 | 35 |
| IGFBP3.FLNVLSPR.+2y4.light | 80 | 2 | 26.2 | 35 |
| IGFBP3.FLNVLSPR.+2y4.heavy | 80 | 2 | 26.2 | 35 |
| IGFBP3.YGQPLPGYTTK.+2y8.light | 120 | 2 | 27 | 50 |
| IGFBP3.YGQPLPGYTTK.+2y8.heavy | 120 | 2 | 27 | 50 |
| SERPINA11.ITPTITNFALR.+2y9+2.light | 60 | 2 | 27.6 | 25 |
| SERPINA11.ITPTITNFALR.+2y9+2.heavy | 60 | 2 | 27.6 | 25 |
| PIGR.VYTVDLGR.+2y6.light | 100 | 2 | 25.6 | 40 |
| PIGR.VYTVDLGR.+2y6.heavy | 100 | 2 | 25.6 | 40 |
| PIGR.VLDSGFR.+2y6.light | 60 | 2 | 22.5 | 35 |
| PIGR.VLDSGFR.+2y6.heavy | 60 | 2 | 22.5 | 35 |
| PON3.STVEIFK.+2y5.light | 60 | 9 | 23.2 | 30 |
| PON3.STVEIFK.+2y5.heavy | 60 | 9 | 23.2 | 30 |
| PON3.YVYVADVAAK.+2y8.light | 80 | 9 | 21.9 | 40 |
| PON3.YVYVADVAAK.+2y8.heavy | 80 | 9 | 21.9 | 40 |
| PON3.ILIGTVFHK.+3y7+2.light | 80 | 9 | 14.5 | 30 |
| PON3.ILIGTVFHK.+3y7+2.heavy | 80 | 9 | 14.5 | 30 |
| SELL.AEIEYLEK.+2y6.light | 80 | 11 | 19.4 | 25 |
| SELL.AEIEYLEK.+2y6.heavy | 80 | 11 | 19.4 | 25 |
| SELL.SYYWIGIR.+2y6.light | 100 | 2 | 28.9 | 35 |
| SELL.SYYWIGIR.+2y6.heavy | 100 | 2 | 28.9 | 35 |
| SERPINA5.DFTFDLYR.+2y6.light | 100 | 15 | 31.4 | 50 |
| SERPINA5.DFTFDLYR.+2y6.heavy | 100 | 15 | 31.4 | 50 |
| SERPINA5.EDQYHYLLDR.+3y3.light | 60 | 9 | 23.7 | 25 |
| SERPINA5.EDQYHYLLDR.+3y3.heavy | 60 | 9 | 23.7 | 25 |
| VWF.EYAPGETVK.+2y6+2.light | 100 | 2 | 23.4 | 40 |
| VWF.EYAPGETVK.+2y6+2.heavy | 100 | 2 | 23.4 | 40 |
| ORM1.SDVVYTDWK.+2y5.light | 60 | 2 | 24.3 | 40 |
| ORM1.SDVVYTDWK.+2y5.heavy | 60 | 2 | 24.3 | 40 |
| PRDX2.ATAVVDGAFK.+2y6.light | 60 | 2 | 21 | 30 |
| PRDX2.ATAVVDGAFK.+2y6.heavy | 60 | 2 | 21 | 30 |
| PRDX2.TDEGIAYR.+2y6.light | 100 | 2 | 25.7 | 40 |
| PRDX2.TDEGIAYR.+2y6.heavy | 100 | 2 | 25.7 | 40 |
| PRDX2.GLFIIDGK.+2y6.light | 100 | 7 | 20.2 | 35 |
| PRDX2.GLFIIDGK.+2y6.heavy | 100 | 7 | 20.2 | 35 |
| SERPINA3.EIGELYLPK.+2y7.light | 80 | 2 | 23 | 45 |
| SERPINA3.EIGELYLPK.+2y7.heavy | 80 | 2 | 23 | 45 |
| SERPINA3.ADLSGITGAR.+2y7.light | 80 | 2 | 26.6 | 50 |
| SERPINA3.ADLSGITGAR.+2y7.heavy | 80 | 2 | 26.6 | 50 |
| LRG1.LHLEGNK.+2y5.light | 100 | 2 | 22.9 | 35 |
| LRG1.LHLEGNK.+2y5.heavy | 100 | 2 | 22.9 | 35 |
| LRG1.DLLLPQPDLR.+2y6.light | 100 | 15 | 29.9 | 45 |
| LRG1.DLLLPQPDLR.+2y6.heavy | 100 | 15 | 29.9 | 45 |
| LRG1.GQTLLAVAK.+2y7.light | 60 | 2 | 19.1 | 45 |
| LRG1.GQTLLAVAK.+2y7.heavy | 60 | 2 | 19.1 | 45 |
| LBP.ITLPDFTGDLR.+2y8.light | 120 | 15 | 35.6 | 50 |
| LBP.ITLPDFTGDLR.+2y8.heavy | 120 | 15 | 35.6 | 50 |
| LBP.LAEGFPLPLLK.+2y8.light | 100 | 15 | 28.4 | 50 |
| LBP.LAEGFPLPLLK.+2y8.heavy | 100 | 15 | 28.4 | 50 |
| TNXB.YEVTVVSVR.+2y7.light | 80 | 9 | 24.8 | 50 |
| TNXB.YEVTVVSVR.+2y7.heavy | 80 | 9 | 24.8 | 50 |
| GSN.TASDFITK.+2y6.light | 80 | 9 | 20.6 | 40 |
| GSN.TASDFITK.+2y6.heavy | 80 | 9 | 20.6 | 40 |
| GSN.AVEVLPK.+2y5.light | 100 | 2 | 17.5 | 30 |
| GSN.AVEVLPK.+2y5.heavy | 100 | 2 | 17.5 | 30 |
| GSN.TGAQELLR.+2y6.light | 100 | 2 | 26.8 | 40 |
| GSN.TGAQELLR.+2y6.heavy | 100 | 2 | 26.8 | 40 |
| CLEC3B.LDTLAQEVALLK.+2y8.light | 100 | 15 | 35.2 | 45 |
| CLEC3B.LDTLAQEVALLK.+2y8.heavy | 100 | 15 | 35.2 | 45 |
| CLEC3B.NWETEITAQPDGGK.+2y5.light | 160 | 9 | 50.9 | 25 |
| CLEC3B.NWETEITAQPDGGK.+2y5.heavy | 160 | 9 | 50.9 | 25 |
| PI16.WDEELAAFAK.+2y5.light | 120 | 15 | 31.9 | 55 |
| PI16.WDEELAAFAK.+2y5.heavy | 120 | 15 | 31.9 | 55 |
| IGFBP2.HGLYNLK.+2y6.light | 100 | 2 | 23.7 | 45 |
| IGFBP2.HGLYNLK.+2y6.heavy | 100 | 2 | 23.7 | 45 |
| GP5.YLGVTLSPR.+2y7.light | 80 | 2 | 25.7 | 50 |
| GP5.YLGVTLSPR.+2y7.heavy | 80 | 2 | 25.7 | 50 |
| VWF.DGTVTTDWK.+2y5.light | 60 | 2 | 22.1 | 35 |
| VWF.DGTVTTDWK.+2y5.heavy | 60 | 2 | 22.1 | 35 |
| VWF.HIVTFDGQNFK.+3b4.light | 60 | 2 | 16.9 | 30 |
| VWF.HIVTFDGQNFK.+3b4.heavy | 60 | 2 | 16.9 | 30 |
| GAPDH.GALQNIIPASTGAAK.+2y9.light | 140 | 7 | 29.6 | 50 |
| GAPDH.GALQNIIPASTGAAK.+2y9.heavy | 140 | 7 | 29.6 | 50 |
| HP.VGYVSGWGR.+2y5.light | 60 | 9 | 27 | 25 |
| HP.VGYVSGWGR.+2y5.heavy | 60 | 9 | 27 | 25 |
| IGFBP2.LIQGAPTIR.+2y7.light | 60 | 2 | 26.8 | 35 |
| IGFBP2.LIQGAPTIR.+2y7.heavy | 60 | 2 | 26.8 | 35 |
| SERPINA5.AVVEVDESGTR.+2y8.light | 100 | 2 | 33.5 | 40 |
| SERPINA5.AVVEVDESGTR.+2y8.heavy | 100 | 2 | 33.5 | 40 |
| TIMP1.GFQALGDAADIR.+2y7.light | 100 | 7 | 25.2 | 35 |
| TIMP1.GFQALGDAADIR.+2y7.heavy | 100 | 7 | 25.2 | 35 |
| HP.HYEGSTVPEK.+3y3.light | 60 | 5 | 18.4 | 40 |
| HP.HYEGSTVPEK.+3y3.heavy | 60 | 5 | 18.4 | 40 |
| ITIH3.EVSFDVELPK.+2y8.light | 120 | 15 | 29.5 | 55 |
| ITIH3.EVSFDVELPK.+2y8.heavy | 120 | 15 | 29.5 | 55 |
| PPIA.VSFELFADK.+2y7.light | 100 | 15 | 26.9 | 45 |
| PPIA.VSFELFADK.+2y7.heavy | 100 | 15 | 26.9 | 45 |

### Examination of Whether Endogenous Peptide is Detected

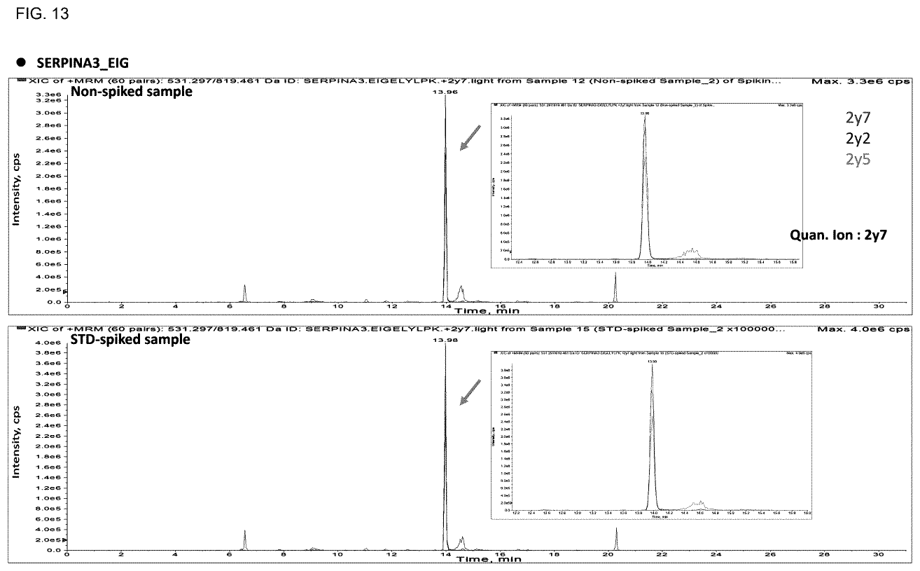

Whether endogenous peptides were detected in serum was examined by applying the optimal LC-MRM MS conditions established through the above-described “Analysis Condition Setting” and “Individual Parameter Optimization” processes. Whether endogenous peptides were detected was examined by spiking serum samples pretreated with peptide standards (FIG. 13).

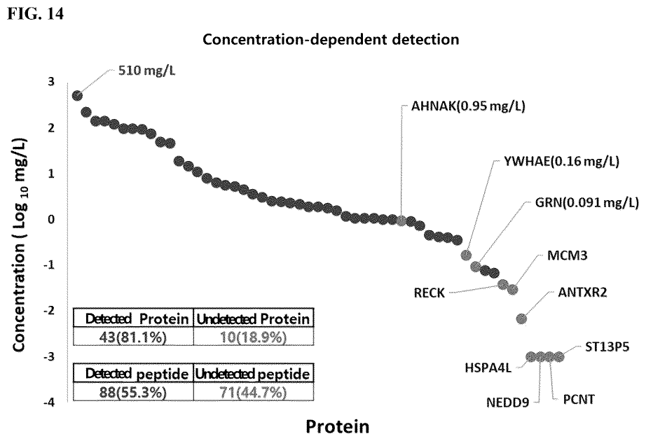

As a result of applying the optimal conditions to examine whether endogenous peptides were detected, it was confirmed that endogenous peptides were detected in 88 peptides for 43 genes among candidate markers corresponding to a total of 53 genes and 159 peptides, indicating a detection rate of 81.1% for the genes. In addition, among the top 3 ions detected, an ion that showed an intensity at which the endogenous peptide was detected more than 10 times the noise, but did not interfere with the peak of the target peptide, was selected for each marker and selected as a quantitative ion (Table 7). Additionally, when the detection of the examined candidate markers was examined by listing the candidate markers according to their blood protein concentration, it was found that most of the undetected markers had relatively low blood concentrations (FIG. 14).

TABLE 7
Endogenous peptide detection and quantitative ions for 150 candidate
marker peptides

| Gene | Sequence | Detection | Quan. Ion |
|---|---|---|---|
| A1BG | SSTSPDR (SEQ ID NO: 100) | O | 2y5 |
| A1BG | LLELTGPK (SEQ ID NO: 99) | O | 2y6 |
| A1BG | ATWSGAVLAGR (SEQ ID NO: | O | 2y8 |
| | 101) | | |
| ALDOA | ELSDIAHR (SEQ ID NO: 107) | O | 3y6+2 |
| ALDOA | ALQASALK (SEQ ID NO: 122) | O | 2y6 |
| ALDOA | GGVVGIK (SEQ ID NO: 156) | X | — |
| AMY2A | NWGEGWGFVPSDR (SEQ ID | O | 2y4 |
| | NO: 108) | | |
| AMY2A | LTGLLDLALEK (SEQ ID NO: | X | — |
| | 157) | | |
| APOA1 | ATEHLSTLSEK (SEQ ID NO: | O | 3y3 |
| | 119) | | |
| APOA1 | DLATVYVDVLK (SEQ ID NO: | O | 2y6 |
| | 117) | | |
| APOA1 | AHVDALR (SEQ ID NO: 118) | O | 2y4 |
| APOA4 | ISASAEELR (SEQ ID NO: 102 | O | 2y8 |
| APOA4 | LAPLAEDVR (SEQ ID NO: | O | 2y5 |
| | 103) | | |
| APOA4 | LTPYADEFK (SEQ ID NO: 2) | O | 2y7+2 |
| APOC3 | DYWSTVK (SEQ ID NO: 104) | O | 2y4 |
| APOC3 | GWVTDGFSSLK (SEQ ID NO: | O | 2y9 |
| | 3) | | |
| APOC3 | VLLVVALLALLASAR (SEQ | X | — |
| | ID NO: 158) | | |
| APOL1 | ALADGVQK (SEQ ID NO: 106) | O | 2y6 |
| APOL1 | NEADELR (SEQ ID NO: 105) | O | 2y5 |
| APOL1 | AEEAGAR (SEQ ID NO: 159) | X | — |
| C9 | ALPTTYEK (SEQ ID NO: 4) | O | 2y6+2 |
| C9 | VVEESELAR (SEQ ID NO: 120) | O | 2y7 |
| C9 | GEIHLGR (SEQ ID NO: 121) | O | 3y5+2 |
| CD14 | ELTLEDLK (SEQ ID NO: 110) | O | 2y6+2 |
| CD14 | VDADADPR (SEQ ID NO: 109) | O | 2y2 |
| CD14 | QYADTVK (SEQ ID NO: 160) | X | — |
| CD163 | GADLSLR (SEQ ID NO: 111) | O | 2y5 |
| CD163 | EAILSHTEK (SEQ ID NO: 161) | X | — |
| CD163 | LTSEASR (SEQ ID NO: 162) | X | — |
| CNDP1 | AIHLDLEEYR (SEQ ID NO: | O | 2y2 |
| | 127) | | |
| CNDP1 | DGSTIPIAK (SEQ ID NO: 129) | O | 2y4 |
| CNDP1 | HLEDVFSK (SEQ ID NO: 128) | O | 3b4 |
| CRP | ESDTSYVSLK (SEQ ID NO: 5) | O | 2y3 |
| CRP | GYSIFSYATK (SEQ ID NO: | O | 2y6 |
| | 112) | | |
| CRP | QDNEILIFWSK (SEQ ID NO: | X | — |
| | 163) | | |
| FCGR3A | AVVFLEPQWYR (SEQ ID NO: | O | 2y5 |
| | 113) | | |
| FCGR3A | YFHHNSDFYIPK (SEQ ID NO: | O | 4y2 |
| | 114) | | |
| HGFAC | TTDVTQTFGIEK (SEQ ID NO: | O | 2y7 |
| | 115) | | |
| HGFAC | EALVPLVADHK (SEQ ID NO: | O | 3y7+2 |
| | 6) | | |
| HGFAC | YEYLEGGDR (SEQ ID NO: | O | 2y7 |
| | 132) | | |
| ICAM1 | LLGIETPLPK (SEQ ID NO: 46) | O | 2y8 |
| ICAM1 | TFLTVYWTPER (SEQ ID NO: | X | — |
| | 164) | | |
| ICAM1 | ATPEDNGR (SEQ ID NO: 165) | X | — |
| IGFBP3 | YGQPLPGYTTK (SEQ ID NO: | O | 2y8 |
| | 136) | | |
| IGFBP3 | FHPLHSK (SEQ ID NO: 134) | O | 3y5+2 |
| IGFBP3 | FLNVLSPR (SEQ ID NO: 135) | O | 2y4 |
| MASP1 | IEPSQAK (SEQ ID NO: 133) | O | 2y5 |
| MASP1 | DSDLLSPSDFK (SEQ ID NO: | X | — |
| | 166) | | |
| MASP1 | FGYILHTDNR (SEQ ID NO: | X | — |
| | 167) | | |
| MCAM | VSPAAPER (SEQ ID NO: 125) | O | 2y6+2 |
| MCAM | EETGQVLER (SEQ ID NO: | X | — |
| | 168) | | |
| MCAM | EAGGGYR (SEQ ID NO: 169) | X | — |
| NOTCH2 | ALGTLLHTNLR (SEQ ID NO: | O | 3y9+2 |
| | 131) | | |
| NOTCH2 | VFLEIDNR (SEQ ID NO:170) | X | — |
| NOTCH2 | VTDLDAR (SEQ ID NO: 171) | X | — |
| PEPD | AFTPFSGPK (SEQ ID NO: 130) | O | 2y7 |
| PEPD | ISSEAHR (SEQ ID NO: 172) | X | — |
| PEPD | VPLALFALNR (SEQ ID NO: | X | — |
| | 173) | | |
| PFN1 | STGGAPTFNVTVTK (SEQ ID | O | 2y9 |
| | NO: 116) | | |
| PFN1 | DSPSVWAAVPGK (SEQ ID | O | 2y3 |
| | NO: 11) | | |
| PFN1 | EGVHGGLINK (SEQ ID NO: | X | — |
| | 174) | | |
| SERPINA11 | ITPTITNFALR (SEQ ID NO: | O | 2y9+2 |
| | 137) | | |
| SERPINA11 | QESFFVDER (SEQ ID NO: 175) | X | — |
| SERPINA11 | VVNPVAG (SEQ ID NO: 176) | X | — |
| SERPINA11 | VGNSLFLDK (SEQ ID NO: | X | — |
| | 177) | | |
| SERPINA3 | ITLLSALVETR (SEQ ID NO: | O | 2y7 |
| | 126) | | |
| SERPINA3 | EIGELYLPK (SEQ ID NO: 14) | O | 2y7 |
| SERPINA3 | ADLSGITGAR (SEQ ID NO: | O | 2y7 |
| | 147) | | |
| PIGR | VPGNVTAVLGETLK (SEQ ID | X | — |
| | NO: 70) | | |
| PIGR | ADAAPDEK (SEQ ID NO: 178) | X | — |
| PIGR | VLDSGFR (SEQ ID NO: 138) | O | 2y6 |
| PIGR | VYTVDLGR (SEQ ID NO: 12) | O | 2y6 |
| CLEC3B | LDTLAQEVALLK (SEQ ID | O | 2y8 |
| | NO: 153) | | |
| CLEC3B | NWETEITAQPDGGK (SEQ ID | O | 2y5 |
| | NO: 154) | | |
| GSN | TASDFITK (SEQ ID NO: 150) | O | 2y6 |
| GSN | AVEVLPK (SEQ ID NO: 151) | O | 2y5 |
| GSN | TGAQELLR (SEQ ID NO: 152) | O | 2y6 |
| LBP | ITLPDFTGDLR (SEQ ID NO: | O | 2y8 |
| | 55) | | |
| LBP | LQGSFDVSVK (SEQ ID NO: | X | — |
| | 179) | | |
| LBP | LAEGFPLPLLK (SEQ ID NO: | O | 2y8 |
| | 56) | | |
| LRG1 | LHLEGNK (SEQ ID NO: 9) | O | 2y5 |
| LRG1 | DLLLPQPDLR (SEQ ID NO: | O | 2y6 |
| | 60) | | |
| LRG1 | GQTLLAVAK (SEQ ID NO: | O | 2y7 |
| | 148) | | |
| ORM1 | ENGTISR (SEQ ID NO: 180) | X | — |
| ORM1 | NWGLSVYADK (SEQ ID NO: | X | — |
| | 181) | | |
| ORM1 | SDVVYTDWK (SEQ ID NO: | O | 2y5 |
| | 10) | | |
| PON3 | ILIGTVFHK (SEQ ID NO: 140) | O | 3y7+2 |
| PON3 | STVEIFK (SEQ ID NO: 139) | O | 2y5 |
| PON3 | YVYVADVAAK (SEQ ID NO: | O | 2y8 |
| | 13) | | |
| PRDX2 | ATAVVDGAFK (SEQ ID NO: | O | 2y6 |
| | 76) | | |
| PRDX2 | TDEGIAYR (SEQ ID NO: 145) | O | 2y6 |
| PRDX2 | GLFIIDGK (SEQ ID NO: 146) | O | 2y6 |
| SELL | AEIEYLEK (SEQ ID NO: 141) | O | 2y6 |
| SELL | DLWNIFK (SEQ ID NO: 182) | X | — |
| SELL | SYYWIGIR (SEQ ID NO: 142) | O | 2y6 |
| TNXB | LEILEELVK (SEQ ID NO: 183) | X | — |
| TNXB | LGELTVTDR (SEQ ID NO: | X | — |
| | 184) | | |
| TNXB | YEVTVVSVR (SEQ ID NO: | O | 2y7 |
| | 149) | | |
| PI16 | ATEASDSR (SEQ ID NO: 185) | X | — |
| PI16 | STHVPIPK (SEQ ID NO: 186) | X | — |
| PI16 | WDEELAAFAK (SEQ ID NO: | O | 2y8 |
| | 155) | | |
| GAPDH | GALQNIIPASTGAAK (SEQ ID | O | 2y9 |
| | NO: 39) | | |
| GAPDH | AAFNSGK (SEQ ID NO: 187) | X | — |
| GAPDH | TVDGPSGK (SEQ ID NO: 188) | X | — |
| GAPDH | VGVNGFGR (SEQ ID NO: 38) | X | — |
| GP5 | TLPAAAFR (SEQ ID NO: 124) | O | 2y6+2 |
| GP5 | YLGVTLSPR (SEQ ID NO: 41) | O | 2y7 |
| GP5 | SIAPGAFDR (SEQ ID NO: 189) | X | — |
| HP | DYAEVGR (SEQ ID NO:123) | O | 2y5 |
| HP | HYEGSTVPEK (SEQ ID NO: | O | 3y3 |
| | 43) | | |
| HP | VGYVSGWGR (SEQ ID NO: | O | 2y5 |
| | 42) | | |
| IGFBP2 | LIQGAPTIR (SEQ ID NO: 7) | O | 2y7 |
| IGFBP2 | HHLGLEEPK (SEQ ID NO: | X | — |
| | 190) | | |
| IGFBP2 | HGLYNLK (SEQ ID NO: 48) | O | 2y6 |
| ITIH3 | SLPEGVANGIEVYSTK (SEQ | O | 2y14+2 |
| | ID NO: 51) | | |
| ITIH3 | ALDLSLK (SEQ ID NO: 8) | O | 2y5 |
| ITIH3 | EVSFDVELPK (SEQ ID NO: | O | 2y8 |
| | 52) | | |
| PPIA | VSFELFADK (SEQ ID NO: 73) | O | 2y7 |
| PPIA | HNGTGGK (SEQ ID NO: 191) | X | — |
| PPIA | FEDENFILK (SEQ ID NO: 74) | X | — |
| SERPINA5 | AVVEVDESGTR (SEQ ID NO: | O | 2y8 |
| | 85) | | |
| SERPINA5 | DFTFDLYR (SEQ ID NO: 143) | O | 2y6 |
| SERPINA5 | EDQYHYLLDR (SEQ ID NO: | O | 3y3 |
| | 86) | | |
| TIMP1 | GFQALGDAADIR (SEQ ID NO: | O | 2y7 |
| | 88) | | |
| TIMP1 | FVGTPEVNQTTLYQR (SEQ ID | X | — |
| | NO: 87) | | |
| TIMP1 | SEEFLIAGK (SEQ ID NO: 192) | X | — |
| VWF | DGTVTTDWK (SEQ ID NO: | O | 2y5 |
| | 93) | | |
| VWF | HIVTFDGQNEK (SEQ ID NO: | O | 3b4 |
| | 94) | | |
| VWF | EYAPGETVK (SEQ ID NO: | O | 2y6+2 |
| | 144) | | |
| AHNAK | FGVSTGR (SEQ ID NO: 193) | X | — |
| AHNAK | APDVDIK (SEQ ID NO: 194) | X | — |
| AHNAK | GPEVDIK (SEQ ID NO: 195) | X | — |
| ANTXR2 | LDGLVPSYAEK (SEQ ID NO: | X | — |
| | 196) | | |
| ANTXR2 | GGFQALK (SEQ ID NO: 197) | X | — |
| ANTXR2 | GSTEEGAR (SEQ ID NO: 198) | X | |
| HSPA4L | LLGTVYEK (SEQ ID NO: 199) | X | — |
| HSPA4L | LFITPEDLSK (SEQ ID NO: | X | — |
| | 200) | | |
| HSPA4L | LVSEIVAK (SEQ ID NO: 201) | X | — |
| NEDD9 | EYLHFVK (SEQ ID NO: 202) | X | — |
| NEDD9 | LSFSSTGSTR (SEQ ID NO: | X | — |
| | 203) | | |
| NEDD9 | LVFIGDTLTR (SEQ ID NO: | X | — |
| | 204) | | |
| GRN | GSEIVAGLEK (SEQ ID NO: | X | — |
| | 205) | | |
| GRN | WPTTLSR (SEQ ID NO: 206) | X | — |
| GRN | SPHVGVK (SEQ ID NO: 207) | X | — |
| PCNT | VETADLK (SEQ ID NO: 208) | X | — |
| PCNT | VLGLETEHK (SEQ ID NO: | X | — |
| | 209) | | |
| PCNT | HAADLGALETR (SEQ ID NO: | X | — |
| | 210) | | |
| RECK | KPITVLEILQK (SEQ ID NO: | X | — |
| | 211) | | |
| RECK | TDSSPGPSQIK (SEQ ID NO: | X | — |
| | 212) | | |
| RECK | LGEASDFIVR (SEQ ID NO: | X | — |
| | 213) | | |
| ST13P5 | ADEPSTEESDLEIDK (SEQ ID | X | — |
| | NO: 214) | | |
| ST13P5 | VAAIEVLNDGELQK (SEQ ID | X | — |
| | NO: 215) | | |
| ST13P5 | AQEEQER (SEQ ID NO: 216) | X | — |
| YWHAE | YLAEFATGNDR (SEQ ID NO: | X | — |
| | 217) | | |
| YWHAE | EDLVYQAK (SEQ ID NO: 218) | X | — |
| YWHAE | IISSIEQK (SEQ ID NO: 219) | X | — |
| MCM3 | EISDHVLR (SEQ ID NO: 220) | X | — |
| MCM3 | TLETLIR (SEQ ID NO: 221) | X | — |
| MCM3 | LATAHAK (SEQ ID NO: 222) | X | — |

### Selection of Isotope and Transition

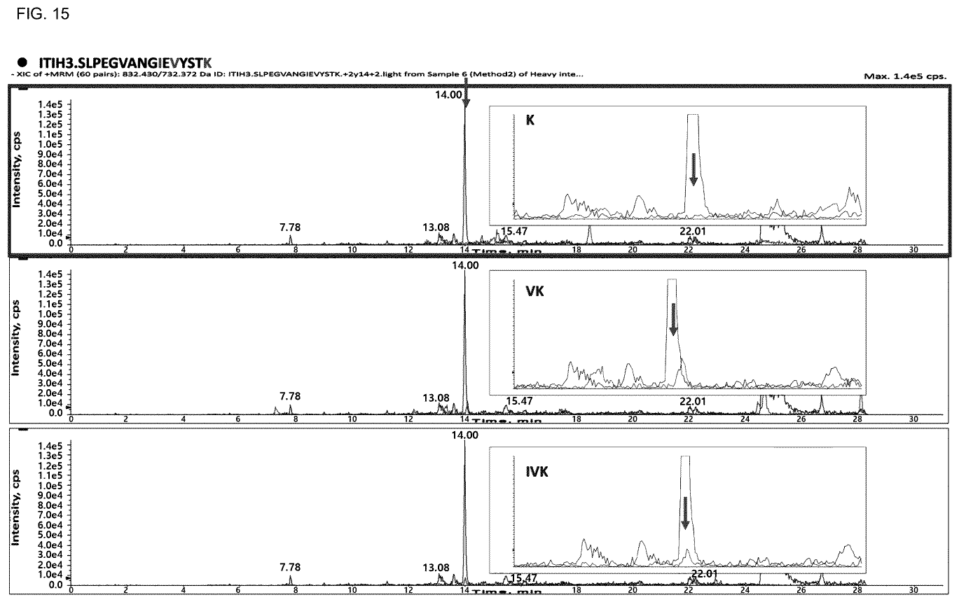

Synthesis of isotope-labeled peptide (heavy) standards was required for method validation and verification analysis for the list of 43 genes and 88 peptides for which endogenous peptides were detected, and a heavy interference test was performed to determine isotopes without interference. The test was performed on serum-pretreated samples by light transition of selected quantitative ions and heavy transition with isotopes (FIG. 15), and an isotope without heavy peak interference in the retention time for each target peptide was finally selected (Table 8).

TABLE 8
Isotopes selected for 88 peptides

| Gene | Sequence¹⁾ | Gene | Sequence¹⁾ |
|---|---|---|---|
| A1BG | SSTSPDR* (SEQ ID NO: 100) | FCGR3A | AVVFLEPQWYR* (SEQ |
| | | | ID NO: 113) |
| A1BG | LLEL*TGPK*(SEQ ID NO: | HGFAC | TTDVTQTFGIEK* (SEQ |
| | 99 | | ID NO: 115) |
| A1BG | ATWSGAVLAGR*(SEQ ID | ITIH3 | SLPEGVANGIEVYSTK |
| | NO: 101) | | * (SEQ ID NO: 51) |
| AMY2A | NWGEGWGF*V*PSDR* | PFN1 | STGGAPTFNV*TV*TK |
| | (SEQ ID NO: 105) | | * (SEQ ID NO: 116) |
| APOA1 | ATEHLSTLSEK*(SEQ ID | PFN1 | DSPSVWAAVPGK* |
| | NO: 119) | | (SEQ ID NO: 11) |
| APOA1 | DLATVYVDVL*K* (SEQ ID | CNDP1 | AIHL*DL*EEYR* (SEQ |
| | NO: 117) | | ID NO: 127) |
| APOA1 | AHVDALR* (SEQ ID NO: | CNDP1 | DGSTIPIA*K* (SEQ ID |
| | 118) | | NO: 129) |
| APOA4 | ISASAEELR* (SEQ ID NO: | CNDP1 | HLEDVFSK* (SEQ ID |
| | 102) | | NO: 128) |
| APOA4 | LAPLAEDVR* (SEQ ID NO: | IGFBP3 | YGQPL*PGYTTK* |
| | 103) | | (SEQ ID NO: 136) |
| APOA4 | LTPYADEFK* (SEQ ID NO: | IGFBP3 | FHPLHSK* (SEQ ID |
| | 2) | | NO: 134) |
| APOC3 | DYWSTVK* (SEQ ID NO: | IGFBP3 | FLNVL*SPR* (SEQ ID |
| | 104) | | NO: 135) |
| APOC3 | GWVTDGFSSLK* (SEQ ID | HGFAC | EALVPLV*A*DHK* |
| | NO: 3) | | (SEQ ID NO: 6) |
| APOL1 | ALA*DGV*QK* (SEQ ID | HGFAC | YEYLEGGDR* (SEQ ID |
| | NO: 114) | | NO: 132) |
| APOL1 | NEADEL*R* (SEQ ID NO: | SERPINA3 | ITLLSALVETR* (SEQ |
| | 115) | | ID NO: 126) |
| C9 | ALPTTYEK* (SEQ ID NO: 4) | ALDOA | ALQASAL*K* (SEQ ID |
| | | | NO: 122) |
| C9 | VVEESELAR* (SEQ ID NO: | GP5 | TLPAAAF*R* (SEQ ID |
| | 120) | | NO: 124) |
| C9 | GEIHLGR* (SEQ ID NO: 121) | MCAM | VSPAA*PER* (SEQ ID |
| | | | NO: 125) |
| CD14 | ELTLEDLK* (SEQ ID NO: | HP | DYAEVGR* (SEQ ID |
| | 110) | | NO:123) |
| CD14 | VDADADPR* (SEQ ID NO: | NOTCH2 | ALGTLLHTNL*R* |
| | 109) | | (SEQ ID NO: 131) |
| CD163 | GADLSLR* (SEQ ID NO: | SERPINA11 | ITPTITNFALR* (SEQ ID |
| | 111) | | NO: 137) |
| CRP | ESDTSYVSLK* (SEQ ID NO: | ITIH3 | ALDLSLK* (SEQ ID |
| | 5) | | NO: 8) |
| CRP | GYSIFSYA*TK* (SEQ ID | MASP1 | IEPSQAK* (SEQ ID NO: |
| | NO: 128) | | 133) |
| PEPD | AFTPF*SGPK* (SEQ ID NO: | SERPINA3 | ADLSGITGAR* (SEQ |
| | 149) | | ID NO: 147) |
| CLEC3B | LDTLAQEVALL*K* (SEQ ID | SERPINA3 | EIGELYLPK* (SEQ ID |
| | NO: 153) | | NO: 14) |
| CLEC3B | NWETEITAQPDGGK* (SEQ | SERPINA5 | DFTFDLYR* (SEQ ID |
| | ID NO: 154) | | NO: 143) |
| GSN | AVEVL*PK* (SEQ ID NO: | SERPINA5 | EDQYHYLL*DR* (SEQ |
| | 165) | | ID NO: 86) |
| GSN | TASDFI*TK* (SEQ ID NO: | TNXB | YEVTVVSV*R* (SEQ |
| | 164) | | ID NO: 149) |
| GSN | TGAQELLR* (SEQ ID NO: | VWF | EYA*PGETV*K* (SEQ |
| | 152) | | ID NO: 144) |
| LBP | ITLPDFTGDLR* (SEQ ID | IGFBP2 | HGLYNL*K (SEQ ID |
| | NO: 55) | | NO: 48) |
| LBP | LAEGFPLPLL*K* (SEQ ID | PI16 | WDEELAAF*AK (SEQ |
| | NO: 56) | | ID NO: 155) |
| LRG1 | DLLL*PQPDL*R* (SEQ ID | ICAM1 | LLGI*ETPL*PK* (SEQ |
| | NO: 60) | | ID NO: 46) |
| LRG1 | GQTLLAVAK* (SEQ ID NO: | ALDOA | ELSDI*A*HR* (SEQ ID |
| | 148) | | NO: 107) |
| LRG1 | LHLEGNK* (SEQ ID NO: 9) | FCGR3A | YFHHNSDFYI*PK* |
| | | | (SEQ ID NO: 114) |
| ORM1 | SDVVYTDWK* (SEQ ID NO: | GP5 | YLGVTLSPR* (SEQ ID |
| | 10) | | NO: 41) |
| PIGR | VLDSGFR* (SEQ ID NO: | VWF | DGTVTTDWK* (SEQ |
| | 138) | | ID NO: 93) |
| PIGR | VYTVDLGR* (SEQ ID NO: | VWF | HIVTFDGQNFK* (SEQ |
| | 12) | | ID NO: 94) |
| PON3 | ILIGTVF*HK* (SEQ ID NO: | GAPDH | GALQNIIPASTGAAK* |
| | 140) | | (SEQ ID NO: 39) |
| PON3 | STVEIF*K* (SEQ ID NO: | HP | VGYVSGWGR* (SEQ |
| | 139) | | ID NO: 42) |
| PON3 | YVYVADVAA*K* (SEQ ID | IGFBP2 | LIQGAPTIR * (SEQ ID |
| | NO: 13) | | NO: 7) |
| PRDX2 | ATAVVDGAFK* (SEQ ID | SERPINA5 | AVVEVDESGTR* (SEQ |
| | NO: 76) | | ID NO: 85) |
| PRDX2 | GLFIIDGK* (SEQ ID NO: | TIMP1 | GFQALGDAADIR* |
| | 146) | | (SEQ ID NO: 88) |
| PRDX2 | TDEGIAYR* (SEQ ID NO: | HP | HYEGSTVPEK* (SEQ |
| | 145) | | ID NO: 43) |
| SELL | AEIEYLEK* (SEQ ID NO: | ITIH3 | EVSFDVELPK* (SEQ |
| | 141) | | ID NO: 52) |
| SELL | SYYWIGIR* (SEQ ID NO: | PPIA | VSFELFADK* (SEQ ID |
| | 142) | | NO: 73) |

### Method Validation

### Purpose of Method Validation

This test was conducted to ensure the reliability of the analysis method by comprehensively evaluating the analytical stability and reproducibility of the biomarkers included in PANCCHECK, a blood test for pancreatic cancer. PANCCHECK is a screening method that can help diagnose pancreatic cancer by injecting 25 human serum biomarkers (A1BG, APOA1, three APOA4, APOC3, APOL1, three C9, CRP, PFN1, HGFAC, GP5, three GSN, LRG1, ORM1, PIGR, PON3, SELL, VWF, SERPINA3, and ITIH3) into LC, performing analysis in MRM (Multiple Reaction Monitoring) mode, and applying the calculated quantitative value to the algorithm to predict pancreatic cancer. In this test, calibration, selectivity, accuracy, precision, matrix effect, carryover, recovery, and stability were evaluated to verify the reliability of the LC-MRM MS quantitative analysis method for the 25 biomarkers to be used for pancreatic cancer screening.

### Reagent Information

The isotope labeled peptide standard (heavy peptide) for each biomarker used in this study was synthesized by and purchased from ANYGEN and BIOSTEM. The stock solution of each heavy peptide was prepared at a concentration of 1 mg/mL using 5% ACN containing 0.1% formic acid (FA), and then stored in a deep freezer at −80° C. and used after diluting with 0.1% FA whenever necessary. Water, acetonitrile (ACN), formic acid (FA), and trifluoroacetic acid (TFA) used in LC-MS/MS analysis were all of LC/MS grade from Thermo Fisher Scientific (USA). Among the reagents used for sample pretreatment, urea and ammonium bicarbonate (ABC) were all products with a purity of 99% or higher, purchased from Sigma-Aldrich (USA), and dithiothreitol (DTT) and iodoacetamide (IAA) were of Bioultra grade from Sigma-Aldrich (USA) and had a purity of 99% or higher. Trypsin for digesting proteins in the sample was V5113 purchased from Promega (USA) (Table 9).

TABLE 9
Information on Standards and Reagents

| | Purity | | |
|---|---|---|---|
| Standard product | (%) | Lot No. | Manufacturer |
| A1BG: LLE{L(13C6, | 98.7 | K231304 | ANYGEN, |
| 15N)}TGP{K(13C6, 15N2)} | | | Korea |
| APOA1: ATEHLSTLSE{K(13C6, 15N2)} | 97.5 | K231307 | |
| APOA4: LTPYADEF{K(13C6, 15N2)} | 98.2 | K231312 | |
| APOA4: ISASAEEL{R(13C6, 15N4)} | 98.0 | K231310 | |
| APOA4: LAPLAEDV{R(13C6, 15N4)} | 97.0 | K231311 | |
| APOC3: GWVTDGFSSL{K(13C6, 15N2)} | 95.3 | K231314 | |
| APOL1: AL{A(13C3, 15N)}DG{V(13C5, | 98.1 | K231315 | |
| 15N)}Q{K(13C6, 15N2)} | | | |
| C9: ALPTTYE{K(13C6, 15N2)} | 98.1 | K231317 | |
| C9: VVEESELA{R(13C6, 15N4)} | 98.5 | K231318 | |
| C9: GEIHLG{R(13C6, 15N4)} | 98.6 | K231319 | |
| CRP: ESDTSYVSL{K(13C6, 15N2)} | 98.5 | K231323 | |
| PFN1: DSPSVWAAVPG{K(13C6, 15N2)} | 98.9 | K231329 | |
| HGFAC: ALVPL{V(13C5, 15N)}{A(13C3, | 95.0 | K231358 | |
| 15N)}DH{K(13C6, 15N2)} | | | |
| GP5: TLPAAA{F(13C9, 15N)}{R(13C6, 15N4)} | 97.6 | K231362 | |
| ITIH3: ALDLSL{K(13C6, 15N2)} | 97.5 | K231367 | |
| GSN: AVEV{L(13C6, 15N)}P{K(13C6, 15N2)} | 96.2 | JT-173112 | BIOSTEM, |
| | | | Korea |
| GSN: TASDF{I(13C6, 15N)}T{K(13C6, 15N2)} | 97.2 | JT-173113 | |
| GSN: TGAQELL{R(13C6, 15N4)} | 97.3 | JT-173114 | |
| LRG1: LHLEGN{K(13C6, 15N2)} | 98.3 | JT-173119 | |
| ORM1: SDVVYTDW{K(13C6, 15N2)} | 97.5 | JT-173120 | |
| PIGR: VYTVDLG{R(13C6, 15N4)} | 97.6 | JT-173122 | |
| PON3: YVYVADVA{A(13C3, 15N)}{K(13C6, 15N2)} | 97.9 | JT-173125 | |
| SELL: AEIEYLE{K(13C6, 15N2)} | 97.6 | JT-173129 | |
| VWF: EY{A(13C3, 15N)}PGET{V(13C5, | 99.1 | JT-173136 | |
| 15N)}{K(13C6, 15N2)} | | | |
| SERPINA3: EIGELYLP{K(13C6, 15N2)} | 97.8 | JT-173132 | |

| Reagents and instruments | Manufacturer |
|---|---|
| Dithiothreitol (DTT) | Sigma, USA |
| Iodoacetamide (IAA) | |
| Ammonium bicarbonate (ABC) | |
| Urea | |
| Trypsin (V5113) | Promega, USA |
| Trifluoroacetic acid (TFA) | Thermo |
| Formic acid (FA) | Fisher, USA |
| Acetonitrile (ACN) | |
| Water | |
| C18 column | Agilent, USA |
| (0.5 × 150 mm, 3.5 μm, 300 Å) | |
| 100 mg C18 96-well plate | Waters, USA |

### Results of Method Validation

Among the 43 genes and 88 peptides for which method validation was performed, peptides corresponding to 19 genes finally passed all of the method validation criteria (Table 10). The results are shown in Table 11 below.

TABLE 10
List of final biomarkers

| No. | Gene | Protein | Sequence | Accession No. |
|---|---|---|---|---|
| 1 | ANPEP | Aminopeptidase N | ALEQALEK (SEQ ID NO: 1) | P15144 |
| 2 | APOA4 | Apolipoprotein A-IV | LTPYADEFK (SEQ ID NO: 2) | P06727 |
| 3 | APOC3 | Apolipoprotein C-III | GWVTDGFSSLK (SEQ ID | P02656 |
| | | | NO: 3) | |
| 4 | C9 | Complement component | ALPTTYEK (SEQ ID NO: 4) | P02748 |
| | | C9 | | |
| 5 | CRP | C-reactive protein | ESDTSYVSLK (SEQ ID NO: | P02741 |
| | | | 5) | |
| 6 | HGFAC | Hepatocyte growth | EALVPLVADHK (SEQ ID | Q04756 |
| | | factor activator | NO: 6) | |
| 7 | IGFBP2 | Insulin-like growth | LIQGAPTIR (SEQ ID NO: 7) | P18065 |
| | | factor-binding protein 2 | | |
| 8 | ITIH3 | Inter-alpha-trypsin | ALDLSLK (SEQ ID NO: 8) | Q06033 |
| | | inhibitor heavy chain H3 | | |
| 9 | LRG1 | Leucine-rich alpha-2- | LHLEGNK (SEQ ID NO: 9) | P02750 |
| | | glycoprotein | | |
| 10 | ORM1 | Alpha-1-acid | SDVVYTDWK (SEQ ID NO: | P02763 |
| | | glycoprotein 1 | 10) | |
| 11 | PFN1 | Profilin-1 | DSPSVWAAVPGK (SEQ ID | P07737 |
| | | | NO: 11) | |
| 12 | PIGR | Polymeric | VYTVDLGR (SEQ ID NO: 12) | P01833 |
| | | immunoglobulin | | |
| | | receptor | | |
| 13 | PON3 | Serum | YVYVADVAAK (SEQ ID | Q15166 |
| | | paraoxonase/lactonase 3 | NO: 13) | |
| 14 | SERPINA3 | Alpha-1- | EIGELYLPK (SEQ ID NO: 14) | P01011 |
| | | antichymotrypsin | | |
| 15 | VWF | von Willebrand factor | ILAGPAGDSNVVK (SEQ ID | P04275 |
| | | | NO: 15) | |

TABLE 11

| | | | | Cali- | | | Matrix | | | | | | | | |
|---|---|---|---|---|---|---|---|---|---|---|---|---|---|---|---|
| | | | | bra- | | | effect | | | Recovery (%) | | | | | |
| | | | LLOQ | tion | Accuracy | Selec- | (CV %) | | Carry- | Low | | Mid | | High | |
| No. | Gene | Sequence | (ng/uL) | R2 | (%) | tivity | Low | High | over(%) | Average | CV | Average | CV | Average | CV |
| 1 | A1BG | LLELTGPK | 0.156 | 0.9999 | 100.0 | Inter- | 2.8 | 3.5 | 2.5 | 92.0 | 10.2 | 94.2 | 3.1 1 | 97.8 | 1.0 |
| | | (SEQ ID | | | | ference | | | | | | | | | |
| | | NO: 99) | | | | ≤ LLOQ | | | | | | | | | |
| | | | | | | 20% | | | | | | | | | |
| 2 | APOA1 | ATEHLST | 5.313 | 0.9997 | 83.3 | | 6.4 | 4.5 | 2.9 | 85.5 | 90 | 86.5 | 5.0 | 97.9 | 2.9 |
| | | LSEK | | | | | | | | | | | | | |
| | | (SEQ ID | | | | | | | | | | | | | |
| | | NO: 119) | | | | | | | | | | | | | |
| 3 | APOA4 | ISASAEELR | 0.313 | 0.9994 | 100.0 | | 6.8 | 3.2 | 1.9 | 89.6 | 3.1 | 88.4 | 6.3 | 96.4 | 3.3 |
| | | (SEQ ID | | | | | | | | | | | | | |
| | | NO: 102 | | | | | | | | | | | | | |
| 4 | APOA4 | LAPLAED | 0.117 | 0.9990 | 100.0 | | 3.0 | 21 | 2.4 | 94.2 | 3.5 | 95.6 | 2.6 | 96.7 | 0.8 |
| | | VR | | | | | | | | | | | | | |
| | | (SEQ ID | | | | | | | | | | | | | |
| | | NO: 103) | | | | | | | | | | | | | |
| 5 | APOA4 | LTPYADE | 0.156 | 0.9999 | 100.0 | | 3.6 | 25 | 1.9 | 92.6 | 6.9 | 96.0 | 1.9 | 96.3 | 3.7 |
| | | FK | | | | | | | | | | | | | |
| | | (SEQ ID | | | | | | | | | | | | | |
| | | NO: 2) | | | | | | | | | | | | | |
| 6 | APOC3 | GWVTDGF | 1.016 | 0.9988 | 100.0 | | 7.7 | 3.7 | 1.7 | 95.6 | 3.5 | 93.8 | 3.2 | 97.0 | 6.0 |
| | | SSLK | | | | | | | | | | | | | |
| | | (SEQ ID | | | | | | | | | | | | | |
| | | NO: 3) | | | | | | | | | | | | | |
| 7 | APOL1 | ALADGVQK | 0.059 | 0.9997 | 100.0 | | 5.4 | 5.8 | 2.6 | 81.8 | 11.4 | 93.7 | 3.0 | 105.4 | 1.5 |
| | | (SEQ ID | | | | | | | | | | | | | |
| | | NO: 106) | | | | | | | | | | | | | |
| 8 | C9 | ALPTTYEK | 0.039 | 0.9999 | 100.0 | | 5.6 | 3.4 | 2.0 | 94.2 | 8.5 | 91.2 | 2.1 | 97.0 | 1.6 |
| | | (SEQ ID | | | | | | | | | | | | | |
| | | NO: 4) | | | | | | | | | | | | | |
| 9 | C9 | VVEESEL | 0.078 | 0.9996 | 100.0 | | 5.5 | 2.5 | 3.0 | 90.1 | 5.6 | 93.1 | 1.7 | 99.1 | 2.3 |
| | | AR | | | | | | | | | | | | | |
| | | (SEQ ID | | | | | | | | | | | | | |
| | | NO: 120) | | | | | | | | | | | | | |
| 10 | C9 | GEIHLGR | 0.039 | 0.9996 | 100.0 | | 6.6 | 5.4 | 4.5 | 83.9 | 11.0 | 93.6 | 5.8 | 97.6 | 1.3 |
| | | (SEQ ID | | | | | | | | | | | | | |
| | | NO: 121) | | | | | | | | | | | | | |
| 11 | CRP | ESDTSY | 0.059 | 0.9995 | 83.3 | | 5.4 | 3.4 | 0.0 | 95.9 | 94 | 91.6 | 1.9 | 107.2 | 0.5 |
| | | VSLK | | | | | | | | | | | | | |
| | | (SEQ ID | | | | | | | | | | | | | |
| | | NO: 5) | | | | | | | | | | | | | |
| 12 | PFN1 | DSPSVWA | 0.098 | 0.9988 | 83.3 | | 3.7 | 4.3 | 3.4 | 98.8 | 5.9 | 92.5 | 4.7 | 99.3 | 4.3 |
| | | AVPGK | | | | | | | | | | | | | |
| | | (SEQ ID | | | | | | | | | | | | | |
| | | NO: 11) | | | | | | | | | | | | | |
| 13 | HGFA | EALVPLV | 0.039 | 0.9996 | 100.0 | | 4.4 | 5.1 | 2.6 | 87.6 | 3.11 | 95.9 | 3.5 | 100.1 | 3.3 |
| | C | ADHK | | | | | | | | | | | | | |
| | | (SEQ ID | | | | | | | | | | | | | |
| | | NO: 6) | | | | | | | | | | | | | |
| 14 | GP5 | TLPAAAFR | 0.005 | 0.9998 | 100.0 | | 7.2 | 3.3 | 18.1 | 89.7 | 10.0 | 96.7 | 3.3 | 94.5 | 1.4 |
| | | (SEQ ID | | | | | | | | | | | | | |
| | | NO: 124) | | | | | | | | | | | | | |
| 15 | GSN | AVEVLPK | 0.117 | 0.9997 | 100.0 | | 2.4 | 32 | 2.1 | 97.3 | 5.9 | 95.0 | 4.2 | 96.0 | 2.1 |
| | | (SEQ ID | | | | | | | | | | | | | |
| | | NO: 151) | | | | | | | | | | | | | |
| 16 | GSN | TASDFITK | 0.117 | 0.9999 | 100.0 | | 4.0 | 3.1 | 2.5 | 96.7 | 4.6 | 97.9 | 3.4 | 97.3 | 3.6 |
| | | (SEQ ID | | | | | | | | | | | | | |
| | | NO: 150) | | | | | | | | | | | | | |
| 17 | GSN | TGAQELLR | 0.273 | 0.9998 | 100.0 | | 4.9 | 4.2 | 2.9 | 96.1 | 3.2 | 99.2 | 1.7 | 96.3 | 5.0 |
| | | (SEQ ID | | | | | | | | | | | | | |
| | | NO: 152) | | | | | | | | | | | | | |
| 18 | LRG1 | LHLEGNK | 0.146 | 0.9999 | 100.0 | | 5.3 | 6.0 | 5.2 | 103.0 | 9.2 | 86.6 | 12.3 | 99.2 | 1.4 |
| | | (SEQ ID | | | | | | | | | | | | | |
| | | NO: 9) | | | | | | | | | | | | | |
| 19 | ORM1 | SDVVYTDWK | 0.469 | 0.9999 | 100.0 | | 3.2 | 63 | 5.6 | 98.2 | 8.7 | 92.8 | 13.8 | 97.1 | 3.2 |
| | | (SEQ ID | | | | | | | | | | | | | |
| | | NO: 10) | | | | | | | | | | | | | |
| 20 | PIGR | VYTVDLGR | 0.020 | 0.9999 | 83.3 | | 4.1 | 3.7 | 0.0 | 105.9 | 112 | 99.4 | 1.3 | 90.7 | 3.5 |
| | | (SEQ ID | | | | | | | | | | | | | |
| | | NO: 12) | | | | | | | | | | | | | |
| 21 | PON3 | YVYVADVAAK | 0.024 | 0.9991 | 83.3 | | 7.5 | 3.0 | 8.9 | 98.4 | 1.8 | 98.2 | 2.7 | 91.3 | 5.6 |
| | | (SEQ ID | | | | | | | | | | | | | |
| | | NO: 13) | | | | | | | | | | | | | |
| 22 | SELL | AEIEYLEK | 0.024 | 0.9999 | 100.0 | | 7.6 | 4.4 | 19.5 | 91.6 | 5.7 | 98.4 | 3.3 | 92.4 | 5.4 |
| | | (SEQ ID | | | | | | | | | | | | | |
| | | NO: 141) | | | | | | | | | | | | | |
| 23 | VWF | EYAPGETVK | 0.044 | 0.9998 | 100.0 | | 8.2 | 67 | 8.7 | 97.8 | 1.5 | 102.1 | 3.2 | 100.3 | 7.9 |
| | | (SEQ ID | | | | | | | | | | | | | |
| | | NO: 144) | | | | | | | | | | | | | |
| 24 | ITIH3 | ALDLSLK | 0.078 | 0.9997 | 100.0 | | 4.3 | 4.4 | 1.4 | 90.2 | 5.1 | 89.1 | 1.8 | 94.9 | 1.4 |
| | | (SEQ ID | | | | | | | | | | | | | |
| | | NO: 8) | | | | | | | | | | | | | |
| 25 | SERPINA3 | EIGELYLPK | 0.625 | 0.9999 | 100.0 | | 7.9 | 5.3 | 2.1 | 101.2 | 2.3 | 97.8 | 2.2 | 93.9 | 3.0 |
| | | (SEQ ID | | | | | | | | | | | | | |
| | | NO: 14) | | | | | | | | | | | | | |

| | |
|---|---|
| Accuracy | Precision |
| (Within-run/ | (Within-run/ |

| | | | Between-run, %) | | | | Between-run, %) | | | | |
|---|---|---|---|---|---|---|---|---|---|---|---|
| No. | Gene | Sequence | LLOQ | Low | Mid | High | LLOQ | Low | Mid | High | Stability Result |
| 1 | A1BG | LLELTGPK | −9.1/ | −2.2/ | −8.3/ | −3.6/ | 1.2/ | 2.7/ | 13.6/ | 3.9/ | Freeze & Thaw stability |
| | | (SEQ ID | −4.9 | −0.8 | −4.5 | −2.4 | 6.3 | 2 | 5.6 | 1.7 | Long term stability |
| | | NO: 99) | | | | | | | | | Short term stability |
| 2 | APOA11 | ATEHLS | −17/ | 4.2/ | 4.7/4 | −4.8/ | 2.6/ | 6.3/ | 1.9/ | 14.5/ | Processed sample stability |
| | | TLSEK | −8.2 | 9.2 | | −2.2 | 13.7 | 6.4 | 0.9 | 3.7 | Accuracy ≤15% |
| | | (SEQ ID | | | | | | | | | |
| | | NO: 119) | | | | | | | | | |
| 3 | APOA4 | ISASAEELR | −11.7/ | 1.4/ | 0.5/ | −3/ | 3.7/ | 3.7/ | 1.9/ | 7.5/ | |
| | | (SEQ ID | −19.3 | −2 | −1.2 | −1.7 | 13.3 | 4.9 | 2.4 | 2 | |
| | | NO: 102) | | | | | | | | | |
| 4 | APOA4 | LAPLAEDVR | 18.6/ | 9.3/ | 4.2/ | −4.8/ | 3.5/ | 1.8/ | 1.6/ | 4.8/ | |
| | | (SEQ ID | 7.6 | 3.8 | −0.3 | −5.2 | 14.5 | 7.6 | 6.4 | 0.6 | |
| | | NO: 103) | | | | | | | | | |
| 5 | APOA4 | LTPYADEFK | 14.4/ | 1.7/ | −2.8/ | −3/ | 1/ | 1.3/ | 4.6/ | 3.7/ | |
| | | (SEQ ID | 9.1 | 0.2 | −2.9 | −2.3 | 6.8 | 2.1 | 0.2 | 1 | |
| | | NO: 2) | | | | | | | | | |
| 6 | APOC3 | GWVTDGFSSLK | 7.7/ | 4.5/ | −0.3/ | 7.4/ | 1.7/ | 5.6/ | 5/ | 5.8/ | |
| | | (SEQ ID | 6.6 | 0.4 | −3.3 | 6.4 | 1.6 | 5.7 | 4.4 | 1.4 | |
| | | NO: 3) | | | | | | | | | |
| 7 | APOL1 | ALADGVQK | 5.1/ | 12.4/ | 3.2/ | −3.5/ | 7.5/ | 7.6/ | 1.8/ | 12.8/ | |
| | | (SEQ ID | 16.2 | 12.8 | 3.5 | −2.6 | 13.5 | 0.4 | 0.4 | 1.3 | |
| | | NO: 106) | | | | | | | | | |
| 8 | C9 | ALPTTYEK | −17.8/ | 4.9/ | 7.3/ | 4.4/ | 3.7/ | 1.4/ | 3.8/ | 7.5/ | |
| | | (SEQ ID | −13.3 | 2.1 | 2.7 | 2.4 | 7.3 | 3.8 | 6.3 | 2.8 | |
| | | NO: 4) | | | | | | | | | |
| 9 | C9 | VVEESELAR | 13.1/ | 6.9/ | 5.3/ | 1.6/ | 3.2/ | 2.4/ | 2.7/ | 3.4/ | |
| | | (SEQ ID | 10.7 | 4.2 | 1.4 | −1.3 | 3 | 3.6 | 5.4 | 4.2 | |
| | | NO: 120) | | | | | | | | | |
| 10 | C9 | GEIHLGR | 14/ | 2.8/ | 3.4/ | −2.3/ | 4.3/ | 6/ | 1.7/ | 8.7/ | |
| | | (SEQ ID | 14.7 | 3.1 | 2 | −1.9 | 0.9 | 0.4 | 1.9 | 0.5 | |
| | | NO: 121) | | | | | | | | | |
| 11 | CRP | ESDTSYVSLK | −13/ | 10.2/ | 0.1/ | −1.4/ | 4.9/ | 6.2/ | 2/ | 7.3/ | |
| | | (SEQ ID | −5.3 | 4.5 | −0.6 | −2.8 | 11.5 | 7.7 | 1 | 2.1 | |
| | | NO: 5) | | | | | | | | | |
| 12 | PFN1 | DSPSVWAAVPGK | 8.1/ | 2.1/ | 0.2/ | −0.7/ | 4/ | 6/ | 6.3/ | 3.9/ | |
| | | (SEQ ID | 9 | 2.4 | −0.8 | −0.4 | 1.2 | 0.5 | 1.4 | 0.4 | |
| | | NO: 11) | | | | | | | | | |
| 13 | HGFAC | EALVPLVADHK | 9.4/ | 2.4/ | −1.7/ | −3/ | 3.4/ | 4.2/ | 7.2/ | 5/ | |
| | | (SEQ ID | −2.3 | −0.9 | −1.4 | −0.8 | 16.9 | 4.7 | 0.4 | 3.1 | |
| | | NO: 6) | | | | | | | | | |
| 14 | GP5 | TLPAAAFR | −16.7/ | 1.6/ | −2.2/ | −4/ | 7.7/ | 3.2/ | 3.5/ | 3.9/ | |
| | | (SEQ ID | −11.7 | −0.8 | −0.8 | −3.1 | 7.9 | 3.5 | 1.9 | 1.4 | |
| | | NO: 124) | | | | | | | | | |
| 15 | GSN | AVEVLPK | 19.4/ | 5.8/ | 4.1/ | −1/ | 2.1/ | 2.6/ | 2.8/ | 3.2/ | |
| | | (SEQ ID | 14.9 | 5 | 5.1 | 1.3 | 5.6 | 1 | 1.4 | 3.2 | |
| | | NO: 151) | | | | | | | | | |
| 16 | GSN | TASDFITK | −4.2/ | 0.4/ | 0.1/ | −0.5/ | 2.8/ | 1.7/ | 2/ | 4/ | |
| | | (SEQ ID | 1.2 | 2.9 | 2.5 | 3.2 | 7.6 | 3.5 | 3.3 | 5.1 | |
| | | NO: 150) | | | | | | | | | |
| 17 | GSN | TGAQELLR | 6.7/ | 4.6/ | −1.2/ | −1/ | 6.2/ | 2.2/ | 1.1/ | 4.9/ | |
| | | (SEQ ID | 16 | 6.7 | 1.4 | −1.2 | 11.4 | 2.8 | 3.7 | 0.2 | |
| | | NO: 152) | | | | | | | | | |
| 18 | LRG1 | LHLEGNK | −1.8/ | 4.3/ | 0.3/ | −1.3/ | 4.6/ | 3.1/ | 5.6/ | 8.1/ | |
| | | (SEQ ID | 7.7 | 2.8 | −1.4 | −5.4 | 12.4 | 2 | 2.4 | 6.1 | |
| | | NO: 9) | | | | | | | | | |
| 19 | ORM1 | SDVVYTDWK | 19.1/ | 5.9/ | | −0.6/ | 2/ | 2.1/ | 4.2/ | 4.7/ | |
| | | (SEQ ID | 10.7 | 3.6 | 2.2/ | 3.3 | 10.7 | 3.2 | 0.1 | 5.4 | |
| | | NO: 10) | | | 2 | | | | | | |
| 20 | PIGR | VYTVDLGR | 7/ | 1.2/ | 1.8/ | −0.9/ | 15.3/ | 8.1/ | 4.2/ | 3.3/ | |
| | | (SEQ ID | 16 | 1.9 | 1.2 | −0.8 | 11 | 1 | 0.8 | 0.1 | |
| | | NO: 12) | | | | | | | | | |
| 21 | PON3 | YVYVADVAAK | 12.3/ | 6.4/ | 1.4/ | 1.7/ | 11.3/ | 2.1/ | 3.3/ | 5.1/ | |
| | | (SEQ ID | 19.9 | 4.9 | 0.1 | −0.2 | 9.1 | 2.1 | 1.8 | 2.7 | |
| | | NO: 13) | | | | | | | | | |
| 22 | SELL | AEIEYLEK | −9.9/ | 3.6/ | 2.8/ | 3.1/ | 13.3/ | 5.8/ | 6.2/ | 4.9/ | |
| | | (SEQ ID | −6.1 | 2.5 | 1.4 | 3.2 | 5.7 | 1.5 | 2 | 0.2 | |
| | | NO: 141) | | | | | | | | | |
| 23 | VWF | EYAPGETVK | 14.7/ | 4/ | 5.6/ | −1.5/ | 15.9/ | 4/ | 6.6/ | 4.1/ | |
| | | (SEQ ID | 1.3 | 2 | 3.7 | 2.3 | 18.7 | 2.6 | 2.7 | 5.2 | |
| | | NO: 144) | | | | | | | | | |
| 24 | ITIH3 | ALDLSLK | 13.3/ | 3.6/ | 6.5/ | 6.4/ | 3.9/ | 3.3/ | 2.8/ | 5.1/ | |
| | | (SEQ ID | 5.9 | 2.4 | 5.4 | 7.5 | 9.9 | 1.7 | 1.4 | 1.4 | |
| | | NO: 8) | | | | | | | | | |
| 25 | SERPINA3 | EIGELYLPK | −11.7/ | −9.7/ | −14.9/ | −0.6/ | 0.9/ | 2.7/ | 4.4/ | 4.7/ | |
| | | (SEQ ID | −7.8 | −11 | −16 | 2.2 | 6.1 | 2.1 | 1.8 | 4 | |
| | | NO: 14) | | | | | | | | | |

### Verification

### Sample Analysis

438 samples were analyzed in MRM mode of LC-MS/MS for the 15 biomarkers finally selected through the method validation test. The samples used were 152 normal samples, 154 pancreatic cancer samples, and 50 benign pancreatic disease samples.

### Sample Information

Serum samples used in this study were randomly selected from the collected samples, and were classified into one sample pool used for verification of calibration, accuracy, precision, and recovery, and six sample pools used for verification of selectivity, matrix effect, stability, etc. The one sample pool was a pool of 22 samples from 9 men and 13 women with an average age of 57.4 years, ranging from 34 to 80 years of age, and included 18 healthy samples, 3 ovarian cancer samples, and 1 stomach cancer sample. The six sample pools included 36 samples from randomly selected healthy individuals, consisted of each of 6 samples, and were collected from 35 men and 1 woman with an average age of 54.1 years, ranging from 32 to 80 years of age.

### Sample Collection Method

To conduct this clinical trial, a total of 724 serum samples collected from 2005 to 2022 among the remaining serum samples stored in the Biobank of Seoul National University Hospital were registered. However, the serum samples that were finally confirmed to meet all of the selection/exclusion criteria for this clinical trial were limited to 438 samples. The normal samples used in this clinical trial were samples collected from people who were confirmed to have no pancreatic cancer through a standard examination at the time of blood collection and who had no history of diagnosis of other cancers, including pancreatic cancer, within the past 10 years from the time of blood collection. Pancreatic cancer samples were samples from patients with pathologically confirmed pancreatic cancer (pancreatic ductal adenocarcinoma type), collected before surgery. The samples were collected from adults over 20 years old, and the distribution of pancreatic cancer samples by stage was confirmed to be 17.5% with AJCC stage 1, 59.1% with AJCC stage 2, 16.2% with AJCC stage 3, and 7.2% with AJCC stage 4. As benign pancreatic disease samples, a total of 50 sera samples were used. Samples collected in the study “Bio-Repository Construction for Liver, Biliary Tract, Pancreas, and Tumor Research (Seoul National University Hospital IRB No. 0901-010-267)” and the study “Genome Analysis of Colon Cancer (Seoul National University Hospital IRB No. 1103-125-357)” were preferentially used, and other samples were obtained from the Biobank of Seoul National University Hospital and used.

TABLE 12

| | Age/ | | | | | |
|---|---|---|---|---|---|---|
| | Other | | | | | |
| | Cancer | Number of | AJCC Stage | | | |
| Group | Type | Samples | 1 | 2 | 3 | 4 |
| Normal | 20-39 | 22 | — | — | — | — |
| | 40-49 | 45 | — | — | — | — |
| | 50-59 | 34 | — | — | — | — |
| | 60-69 | 30 | — | — | — | — |
| | 70+ | 21 | — | — | — | — |
| Total | | 152 | | | | |
| Pancreatic | 20-39 | 2 | — | 1 | — | 1 |
| Ductal | 40-49 | 7 | 3 | 4 | — | — |
| Adenocarcinoma | 50-59 | 28 | 7 | 15 | 4 | 2 |
| | 60-69 | 59 | 6 | 39 | 12 | 2 |
| | 70+ | 58 | 11 | 32 | 9 | 6 |

| | |
|---|---|
| Total | 154 |
| Benign Pancreatic Disease | 50 |
| Grand total | 356 |

### 3. Development of Marker Panel

### MRM Mass Spectrometry

### Data Exploratory Analysis

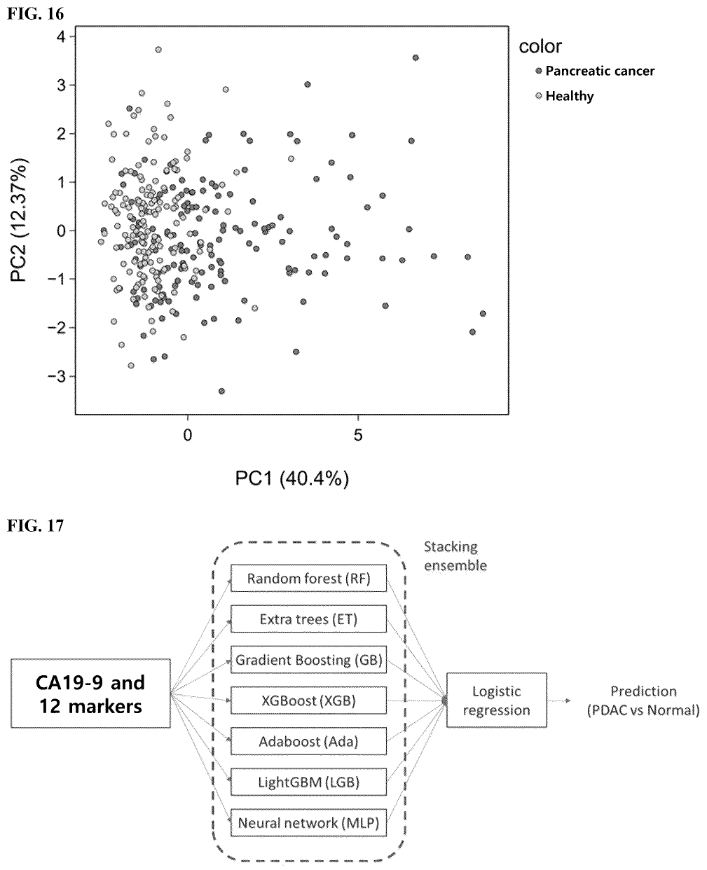

The analyzed quantitative values are used as input data, and basic exploratory analysis was conducted to select a more efficient deep learning/machine learning model by analyzing the type, nature, and number of data. As a result of the PCA analysis, a pattern in which the groups of pancreatic cancer and healthy people are divided based on PC1 can be seen (FIG. 16).

### Selection of 12 Markers

In order to exclude markers that are less important in distinguishing between pancreatic cancer and healthy people, among the 15 markers, 5-fold cross-validation was added to the LGBM model and the results of each validation were examined. Table 13 below is a table ranking important markers in each fold, and three markers (APOC3, CRP, and ORM1) ranked low in five folds were excluded. Using the final 12 markers (Table 14), a model was developed and performance thereof was tested using a machine learning algorithm.

TABLE 13
Results of 5-fold cross-validation

| No. | Gene | Sequence | Fold1 | Fold2 | Fold3 | Fold4 | Fold5 |
|---|---|---|---|---|---|---|---|
| 1 | ANPEP | ALEQALEK (SEQ ID | 3 | 7 | 11 | 3 | 10 |
| | | NO: 1) | | | | | |
| 2 | APOA4 | LTPYADEFK (SEQ | 2 | 2 | 1 | 6 | 2 |
| | | ID NO: 2) | | | | | |
| 3 | APOC3 | GWVTDGFSSLK | 10 | 5 | 7 | 13 | 5 |
| | | (SEQ ID NO: 3) | | | | | |
| 4 | C9 | ALPTTYEK (SEQ ID | 7 | 10 | 9 | 12 | 13 |
| | | NO: 4) | | | | | |
| 5 | CRP | ESDTSYVSLK (SEQ | 15 | 13 | 15 | 10 | 9 |
| | | ID NO: 5) | | | | | |
| 6 | HGFAC | EALVPLVADHK | 12 | 6 | 12 | 5 | 10 |
| | | (SEQ ID NO: 6) | | | | | |
| 7 | IGFBP2 | LIQGAPTIR (SEQ ID | 8 | 4 | 14 | 14 | 1 |
| | | NO: 7) | | | | | |
| 8 | ITIH3 | ALDLSLK (SEQ ID | 4 | 8 | 6 | 7 | 8 |
| | | NO: 8) | | | | | |
| 9 | LRG1 | LHLEGNK (SEQ ID | 14 | 14 | 13 | 8 | 15 |
| | | NO: 9) | | | | | |
| 10 | ORM1 | SDVVYTDWK (SEQ | 11 | 12 | 10 | 15 | 14 |
| | | ID NO: 10) | | | | | |
| 11 | PFN1 | DSPSVWAAVPGK | 6 | 11 | 2 | 11 | 5 |
| | | (SEQ ID NO: 11) | | | | | |
| 12 | PIGR | VYTVDLGR (SEQ | 13 | 3 | 8 | 8 | 12 |
| | | ID NO: 12) | | | | | |
| 13 | PON3 | YVYVADVAAK | 1 | 1 | 3 | 1 | 3 |
| | | (SEQ ID NO: 13) | | | | | |
| 14 | SERPINA3 | EIGEL YLPK (SEQ | 5 | 15 | 4 | 2 | 7 |
| | | ID NO: 14) | | | | | |
| 15 | VWF | ILAGPAGDSNVVK | 9 | 9 | 5 | 3 | 4 |
| | | (SEQ ID NO: 15) | | | | | |

TABLE 14
12 markers used in algorithm development

| | | | | Accession |
|---|---|---|---|---|
| No. | Gene | Protein | Sequence | No. |
| 1 | ANPEP | Aminopeptidase N | ALEQALEK (SEQ | P15144 |
| | | | ID NO: 1) | |
| 2 | APOA4 | Apolipoprotein A-IV | LTPYADEFK (SEQ | P06727 |
| | | | ID NO: 2) | |
| 3 | C9 | Complement component C9 | ALPTTYEK (SEQ | P02748 |
| | | | ID NO: 4) | |
| 4 | HGFAC | Hepatocyte growth factor | EALVPLVADHK | Q04756 |
| | | activator | (SEQ ID NO: 6) | |
| 5 | IGFBP2 | Insulin-like growth factor- | LIQGAPTIR (SEQ | P18065 |
| | | binding protein 2 | ID NO: 7) | |
| 6 | ITIH3 | Inter-alpha-trypsin inhibitor | ALDLSLK (SEQ ID | Q06033 |
| | | heavy chain H3 | NO: 8) | |
| 7 | LRG1 | Leucine-rich alpha-2- | LHLEGNK (SEQ ID | P02750 |
| | | glycoprotein | NO: 9) | |
| 8 | PFN1 | Profilin-1 | DSPSVWAAVPGK | P07737 |
| | | | (SEQ ID NO: 11) | |
| 9 | PIGR | Polymeric immunoglobulin | VYTVDLGR (SEQ | P01833 |
| | | receptor | ID NO: 12) | |
| 10 | PON3 | Serum paraoxonase/lactonase | YVYVADVAAK | Q15166 |
| | | 3 | (SEQ ID NO: 13) | |
| 11 | SERPINA3 | Alpha-1-antichymotrypsin | EIGELYLPK (SEQ | P01011 |
| | | | ID NO: 14) | |
| 12 | VWF | von Willebrand factor | ILAGPAGDSNVVK | P04275 |
| | | | (SEQ ID NO: 15) | |

### Algorithm Development

### Data Used to Develop Prediction Model

The samples used for model training and validation were a total of 415 samples, including healthy samples, pancreatic cancer samples, benign pancreatic disease samples, and other cancer samples (Table 15). Predictor variables include CA19-9 levels and concentrations of 12 protein markers.

TABLE 15

| | |
|---|---|
| #samples | ratio (%) |

| | | | | | |
|---|---|---|---|---|---|
| PDAC | stage-1 | 27 | 154 | 6.5% | 37.1% |
| | stage-2 | 91 | | 21.9% | |
| | stage-3 | 25 | | 6.0% | |
| | stage-4 | 11 | | 2.7% | |

| | | |
|---|---|---|
| Benign | 50 | 12.0% |
| Healthy | 152 | 36.6% |

| | | | | |
|---|---|---|---|---|
| Stomach | 20 | 59 | 4.8% | 14.2% |
| Liver | 20 | | 4.8% | |
| Colorectal | 19 | | 4.6% | |

| | | |
|---|---|---|
| Total | 415 | 100.0% |

### Prediction Model

A prediction model was developed as a stacking ensemble model by combining a total of seven machine learning algorithms to maximize model robustness while providing optimal performance (FIG. 17). This model is a binary classification model that classifies pancreatic cancer, and the component algorithms of the ensemble include tree-based models (Extra-trees (ET), Light-GBM (LGBM), random-forest (RF), gradient-boosting (GB), XGBoost (XGB), and Ada-boost (Ada) algorithms) and neural network-based multi-layer perceptron (MLP). Default values are used for hyperparameters of individual algorithms, and the MLP model may consist of one hidden layer consisting of a total of 16 neurons. The final prediction result is evaluated by combining the prediction results of individual models using a logistic regression algorithm.

### Model Training and Validation

The prediction model was trained and validated using a 5-fold cross-validation method, and the performance thereof was compared with those of an individual model of the stacking model and a logistic regression model using only CA19-9 values.

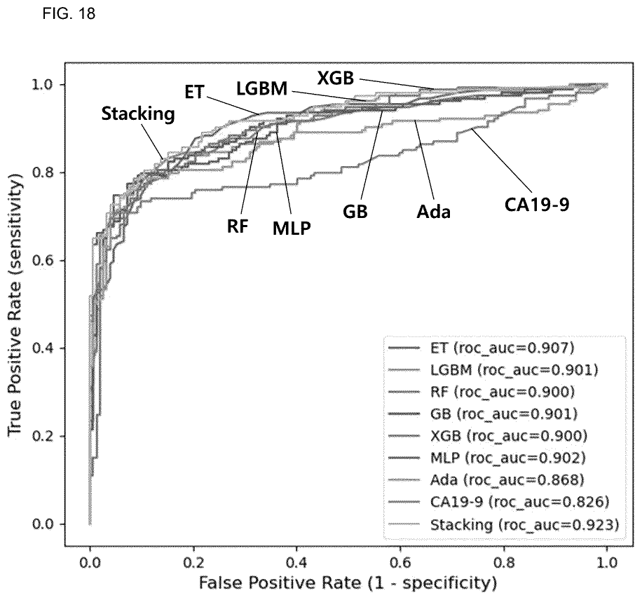

In the performance comparison test, the prediction models including 12 markers showed better performance (AUC-0.868 to 0.923) than CA19-9 (AUC=0.826), and the stacking ensemble model designed as the prediction model showed better performance than the individual algorithms (FIG. 18). The prediction model (stacking ensemble) using the 12 markers showed higher specificity than CA19-9 in the high sensitivity area (Table 16).

The threshold value was estimated based on when the sensitivity of the model was 84, and the sensitivity of the model depending on pancreatic cancer stage and the specificity of the model for benign pancreatic disease and other cancer samples were evaluated. The sensitivity tended to increase as the stage of pancreatic cancer increased (Table 17), and the specificity ranged from 60 to 90% for other cancers and benign pancreatic disease (Table 18).

TABLE 16
Specificity of each model for certain sensitivity

| | ROC |
|---|---|
| | AUC |
| 12 markers + CA19-9 | 0.92 |
| CA19-9 | 0.83 |

TABLE 17
Sensitivity of prediction model depending
on pancreatic cancer stage

84.4% sensitivity

| | |
|---|---|
| Pancreatic | Predicted value |

| cancer | Number of | | Pancreatic | |
|---|---|---|---|---|
| stage | samples | Healthy | cancer | Sensitivity |
| 1 | 27 | 9 | 18 | 0.67 |
| 2 | 91 | 10 | 81 | 0.89 |
| 3 | 25 | 2 | 23 | 0.92 |
| 4 | 11 | 3 | 8 | 0.73 |

TABLE 18

| | | 84.4% sensitivity | | |
|---|---|---|---|---|
| | | Predicted value | | |
| | Number of | | Pancreatic | |
| | samples | Healthy | cancer | Specificity |
| Benign pancreatic | 50 | 32 | 18 | 0.64 |
| disease | | | | |
| Healthy | 152 | 129 | 23 | 0.85 |

### 4. Conclusion

In this study, differentially expressed proteins between normal and patient groups were identified through LC-DIA MS analysis of serum samples without depletion of highly abundant proteins, and the final 12 markers (ANPEP, APOA4, C9, HGFAC, IGFBP2, ITIH3, LRG1, PFN1, PIGR, PON3, SERPINA3, and VWF) that passes through method validation and verification analysis through LC-MRM MS for optimization of the analysis method were selected as a diagnostic marker panel for pancreatic cancer. In addition, the algorithm formula was developed and validated, and the combination of CA19-9, which is currently used to diagnose pancreatic cancer, and 12 proteins discovered through this study showed a high accuracy of 0.92 based on AUC, suggesting that the combination is a marker combination suitable for direct clinical application.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

### SEQUENCE LISTING

Sequence list electronic file attached (D:\Solusseum\Users\p04\Desktop\PDPB234295.xml)

1. A composition for diagnosing pancreatic cancer, comprising, as an active ingredient, an agent for measuring an expression level of at least one polypeptide selected from the group consisting of ANPEP (aminopeptidase N), APOA4 (apolipoprotein A-IV), APOC3 (apolipoprotein C-III), C9 (complement component C9), CRP (C-reactive protein), HGFAC (hepatocyte growth factor activator), IGFBP2 (insulin-like growth factor-binding protein 2), ITIH3 (inter-alpha-trypsin inhibitor heavy chain H3), LRG1 (leucine-rich alpha-2-glycoprotein), ORM1 (alpha-1-acid glycoprotein 1), PFN1 (profilin-1), PIGR (polymeric immunoglobulin receptor), PON3 (serum paraoxonase/lactonase 3), SERPINA3 (alpha-1-antichymotrypsin), and VWF (von Willebrand factor), or a fragment thereof, or a gene encoding the polypeptide or fragment thereof.

2. The composition of claim 1, wherein
the fragment of the ANPEP polypeptide has the amino acid sequence of SEQ ID NO: 1 (ALEQALEK);
the fragment of the APOA4 polypeptide has the amino acid sequence of SEQ ID NO: 2 (LTPYADEFK);
the fragment of the APOC3 polypeptide has the amino acid sequence of SEQ ID NO: 3 (GWVTDGFSSLK);
the fragment of the C9 polypeptide has the amino acid sequence of SEQ ID NO: 4 (ALPTTYEK);
the fragment of the CRP polypeptide has the amino acid sequence of SEQ ID NO: 5 (ESDTSYVSLK);
the fragment of the HGFAC polypeptide has the amino acid sequence of SEQ ID NO: 6 (EALVPLVADHK);
the fragment of the IGFBP2 polypeptide has the amino acid sequence of SEQ ID NO: 7 (LIQGAPTIR);
the fragment of the ITIH3 polypeptide has the amino acid sequence of SEQ ID NO: 8 (ALDLSLK);
the fragment of the LRG1 polypeptide has the amino acid sequence of SEQ ID NO: 9 (LHLEGNK);
the fragment of the ORM1 polypeptide has the amino acid sequence of SEQ ID NO: 10 (SDVVYTDWK);
the fragment of the PFN1 polypeptide has the amino acid sequence of SEQ ID NO: 11 (DSPSVWAAVPGK);
the fragment of the PIGR polypeptide has the amino acid sequence of SEQ ID NO: 12 (VYTVDLGR);
the fragment of the PON3 polypeptide has the amino acid sequence of SEQ ID NO: 13 (YVYVADVAAK);
the fragment of the SERPINA3 polypeptide has the amino acid sequence of SEQ ID NO: 14 (EIGELYLPK); and
the fragment of the VWF polypeptide has the amino acid sequence of SEQ ID NO: 15 (ILAGPAGDSNVVK).

3. The composition of claim 1, wherein a subject with pancreatic cancer has an increased expression level of the at least one gene selected from the group consisting of ANPEP, C9, CRP, IGFBP2, ITIH3, LRG1, ORM1, PIGR, SERPINA3 and VWF, or the protein encoded thereby, and has a decreased expression level of the at least one gene selected from the group consisting of APOA4, APOC3, HGFAC, PFN1 and PON3, or the protein encoded thereby.

4. The composition of claim 1, wherein the pancreatic cancer is pancreatic ductal adenocarcinoma (PDAC).

5. The composition of claim 1, wherein the agent for measuring the expression level of the polypeptide comprises at least one selected from the group consisting of an antibody, an antigen-binding fragment, a ligand, a peptide nucleic acid (PNA), and an aptamer, which bind specifically to the polypeptide or fragment thereof.

6. The composition of claim 1, wherein the agent for measuring the expression level of the gene encoding the polypeptide or fragment thereof comprises at least one selected from the group consisting of a primer, a probe, and an antisense oligonucleotide, which bind specifically to the gene.

7. A diagnostic kit comprising the composition of claim 1.

8.-21. (canceled)

22. A method for screening a composition for preventing or treating pancreatic cancer, comprising steps of:
(a) bringing a test substance into contact with a biological sample containing ANPEP, APOA4, APOC3, C9, CRP, HGFAC, IGFBP2, ITIH3, LRG1, ORM1, PFN1, PIGR, PON3, SERPINA3 and VWF genes, or proteins encoded by the genes, or cells expressing the genes or proteins; and
(b) measuring expression levels of the proteins or the genes in the biological sample,
wherein, if the activities or expression levels of the ANPEP, C9, CRP, IGFBP2, ITIH3, LRG1, ORM1, PIGR, SERPINA3 and VWF genes or proteins in the biological sample decreased, or
if the activities or expression levels of the APOA4, APOC3, HGFAC, PFN1 and PON3 genes or proteins in the biological sample increased,
the test substance is determined as the composition for preventing or treating pancreatic cancer.

23. The method of claim 22, wherein the pancreatic cancer is pancreatic ductal adenocarcinoma (PDAC).

24. A system for diagnosing pancreatic cancer, comprising:
an input unit configured to receive an input value;
a reading unit comprising a machine learning model pre-trained to read whether pancreatic cancer has occurred; and
an output unit configured to output whether pancreatic cancer has occurred,
wherein the input value is a measured value for an expression level of at least one polypeptide selected from the group consisting of SEQ ID NO: 1 (ALEQALEK), SEQ ID NO: 2 (LTPYADEFK), SEQ ID NO: 3 (GWVTDGFSSLK), SEQ ID NO: 4 (ALPTTYEK), SEQ ID NO: 5 (ESDTSYVSLK), SEQ ID NO: 6 (EALVPLVADHK), SEQ ID NO: 7 (LIQGAPTIR), SEQ ID NO: 8 (ALDLSLK), SEQ ID NO: 9 (LHLEGNK), SEQ ID NO: 10 (SDVVYTDWK), SEQ ID NO: 11 (DSPSVWAAVPGK), SEQ ID NO: 12 (VYTVDLGR), SEQ ID NO: 13 (YVYVADVAAK), SEQ ID NO: 14 (EIGELYLPK), and SEQ ID NO: 15 (ILAGPAGDSNVVK), in a biological sample.

25. The system of claim 24, wherein the machine learning model is a deep learning model.

26. The system of claim 24, wherein the biological sample is whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid.

27. The system of claim 24, wherein the measured value for the expression level of the polypeptide is a quantitative value obtained by mass spectrometry.

28. (canceled)

29. The system of claim 24, wherein the pancreatic cancer is pancreatic ductal adenocarcinoma (PDAC).

30. The system of claim 24, wherein
a mass-to-charge ratio (m/z) of the polypeptide represented by SEQ ID NO: 1, when the z value is 1, is 901.506 or 901.506±1 for a light peptide, and 909.52 or 909.52±1 for a heavy peptide;
a mass-to-charge ratio (m/z) of the polypeptide represented by SEQ ID NO: 2, when the z value is 1, is 1083.536 or 1083.536±1 for a light peptide, and 1091.550 or 1091.550±1 for a heavy peptide;
a mass-to-charge ratio (m/z) of the polypeptide represented by SEQ ID NO: 3, when the z value is 1, is 1196.595 or 1196.595±1 for a light peptide, and 1204.609 or 1204.609±1 for a heavy peptide;
a mass-to-charge ratio (m/z) of the polypeptide represented by SEQ ID NO: 4, when the z value is 1, is 922.496 or 922.496±1 for a light peptide, and 930.508 or 930.508±1 for a heavy peptide;
a mass-to-charge ratio (m/z) of the polypeptide represented by SEQ ID NO: 5, when the z value is 1, is 1128.542 or 1128.542±1 for a light peptide, and 1136.556 or 1136.556±1 for a heavy peptide;
a mass-to-charge ratio (m/z) of the polypeptide represented by SEQ ID NO: 6, when the z value is 1, is 1191.673 or 1191.673±1 for a light peptide, and 1209.708 or 1209.708±1 for a heavy peptide;
a mass-to-charge ratio (m/z) of the polypeptide represented by SEQ ID NO: 7, when the z value is 1, is 968.596 or 968.596±1 for a light peptide, and 978.604 or 978.604±1 for a heavy peptide;
a mass-to-charge ratio (m/z) of the polypeptide represented by SEQ ID NO: 8, when the z value is 1, is 759.468 or 759.468±1 for a light peptide, and 767.482 or 767.482±1 for a heavy peptide;
a mass-to-charge ratio (m/z) of the polypeptide represented by SEQ ID NO: 9, when the z value is 1, is 810.454 or 810.454±1 for a light peptide, and 818.468 or 818.468±1 for a heavy peptide;
a mass-to-charge ratio (m/z) of the polypeptide represented by SEQ ID NO: 10, when the z value is 1, is 1112.526 or 1112.526±1 for a light peptide, and 1120.540 or 1120.540±1 for a heavy peptide;
a mass-to-charge ratio (m/z) of the polypeptide represented by SEQ ID NO: 11, when the z value is 1, is 1213.621 or 1213.621±1 for a light peptide, and 1221.635 or 1221.635±1 for a heavy peptide;
a mass-to-charge ratio (m/z) of the polypeptide represented by SEQ ID NO: 12, when the z value is 1, is 922.506 or 922.506±1 for a light peptide, and 932.508 or 932.508±1 for a heavy peptide;
a mass-to-charge ratio (m/z) of the polypeptide represented by SEQ ID NO: 13, when the z value is 1, is 1098.59 or 1098.59±1 for a light peptide, and 1110.61 or 1110.61±1 for a heavy peptide;
a mass-to-charge ratio (m/z) of the polypeptide represented by SEQ ID NO: 14, when the z value is 1, is 1061.588 or 1061.588±1 for a light peptide, and 1069.602 or 1069.602±1 for a heavy peptide; and
a mass-to-charge ratio (m/z) of the polypeptide represented by SEQ ID NO: 15, when the z value is 1, is 1240.696 or 1240.696±1 for a light peptide, and 1248.71 or 1248.71±1 for a heavy peptide.

31. The system of claim 24, wherein the multiple-reaction monitoring is performed using, as an internal standard substance, either a synthetic peptide obtained by substituting a predetermined element of a predetermined amino acid in each of the polypeptides with an isotope, or E. coli beta-galactosidase.

32. The system of claim 31, wherein the synthetic peptide has the same sequence as the sequence represented by SEQ ID NO: 1, 2, 3, 4, 5, 6, 7, 8, 9, 10, 11, 12, 13, 14, or 15, and contains a stable isotope.

33. The system of claim 32, wherein the stable isotope is a stable isotope of any one or more elements selected from the group consisting of carbon and nitrogen.

34. The system of claim 24, wherein,
wherein the measured expression level of the ANPEP (aminopeptidase N), C9 (complement component C9), CRP (C-reactive protein), IGFBP2 (insulin-like growth factor-binding protein 2), ITIH3 (inter-alpha-trypsin inhibitor heavy chain H3), LRG1 (leucine-rich alpha-2-glycoprotein), ORM1 (alpha-1-acid glycoprotein 1), PIGR (polymeric immunoglobulin receptor), SERPINA3 (alpha-1-antichymotrypsin) or VWF (von Willebrand factor) polypeptide or the gene encoding the same in the biological sample isolated from the subject of interest is higher than that in a normal control group, or
wherein the measured expression level of the APOA4 (apolipoprotein A-IV), APOC3 (apolipoprotein C-III), HGFAC (hepatocyte growth factor activator), PFN1 (profilin-1) or PON3 (serum paraoxonase/lactonase 3) polypeptide or the gene encoding the same in the biological sample is lower than that in the normal control group,
a likelihood of developing the pancreatic cancer is predicted to be high.

35. The system of claim 24, wherein the pancreatic cancer is pancreatic ductal adenocarcinoma (PDAC).


SEQUENCE LISTING (222 sequences)

SEQ ID NO: 1 - protein, 8 aa, synthetic construct
ALEQALEK

SEQ ID NO: 2 - protein, 9 aa, synthetic construct
LTPYADEFK

SEQ ID NO: 3 - protein, 11 aa, synthetic construct
GWVTDGFSSLK

SEQ ID NO: 4 - protein, 8 aa, synthetic construct
ALPTTYEK

SEQ ID NO: 5 - protein, 10 aa, synthetic construct
ESDTSYVSLK

SEQ ID NO: 6 - protein, 11 aa, synthetic construct
EALVPLVADHK

SEQ ID NO: 7 - protein, 9 aa, synthetic construct
LIQGAPTIR

SEQ ID NO: 8 - protein, 7 aa, synthetic construct
ALDLSLK

SEQ ID NO: 9 - protein, 7 aa, synthetic construct
LHLEGNK

SEQ ID NO: 10 - protein, 9 aa, synthetic construct
SDVVYTDWK

SEQ ID NO: 11 - protein, 12 aa, synthetic construct
DSPSVWAAVPGK

SEQ ID NO: 12 - protein, 8 aa, synthetic construct
VYTVDLGR

SEQ ID NO: 13 - protein, 10 aa, synthetic construct
YVYVADVAAK

SEQ ID NO: 14 - protein, 9 aa, synthetic construct
EIGELYLPK

SEQ ID NO: 15 - protein, 13 aa, synthetic construct
ILAGPAGDSNVVK

SEQ ID NO: 16 - protein, 12 aa, synthetic construct
ADDGRPFPQVIK

SEQ ID NO: 17 - protein, 8 aa, synthetic construct
AAQEEYVK

SEQ ID NO: 18 - protein, 11 aa, synthetic construct
AEFNITLIHPK

SEQ ID NO: 19 - protein, 10 aa, synthetic construct
NATLVNEADK

SEQ ID NO: 20 - protein, 12 aa, synthetic construct
FQNSAILTIQPK

SEQ ID NO: 21 - protein, 7 aa, synthetic construct
VFQFLEK

SEQ ID NO: 22 - protein, 20 aa, synthetic construct
LIDQYGTHYLQSGSLGGEYR

SEQ ID NO: 23 - protein, 9 aa, synthetic construct
LTPLYELVK

SEQ ID NO: 24 - protein, 12 aa, synthetic construct
AEDTVTVENVLK

SEQ ID NO: 25 - protein, 9 aa, synthetic construct
ELASLHGTK

SEQ ID NO: 26 - protein, 12 aa, synthetic construct
FVFETPFTLSGK

SEQ ID NO: 27 - protein, 10 aa, synthetic construct
LTGLSEISQR

SEQ ID NO: 28 - protein, 10 aa, synthetic construct
TSEGFQAFSK

SEQ ID NO: 29 - protein, 12 aa, synthetic construct
TVEDAGQYLHQK

SEQ ID NO: 30 - protein, 14 aa, synthetic construct
TASGDYIDSSWELR

SEQ ID NO: 31 - protein, 13 aa, synthetic construct
VVLAGGVAPALFR

SEQ ID NO: 32 - protein, 11 aa, synthetic construct
GSESGIFTNTK

SEQ ID NO: 33 - protein, 15 aa, synthetic construct
ESSSHHPGIAEFPSR

SEQ ID NO: 34 - protein, 13 aa, synthetic construct
QGFGNVATNTDGK

SEQ ID NO: 35 - protein, 14 aa, synthetic construct
AHYGGFTVQNEANK

SEQ ID NO: 36 - protein, 15 aa, synthetic construct
IHLISTQSAIPYALR

SEQ ID NO: 37 - protein, 13 aa, synthetic construct
YEASILTHDSSIR

SEQ ID NO: 38 - protein, 8 aa, synthetic construct
VGVNGFGR

SEQ ID NO: 39 - protein, 15 aa, synthetic construct
GALQNIIPASTGAAK

SEQ ID NO: 40 - protein, 11 aa, synthetic construct
LPNLSSLTLSR

SEQ ID NO: 41 - protein, 9 aa, synthetic construct
YLGVTLSPR

SEQ ID NO: 42 - protein, 9 aa, synthetic construct
VGYVSGWGR

SEQ ID NO: 43 - protein, 10 aa, synthetic construct
HYEGSTVPEK

SEQ ID NO: 44 - protein, 12 aa, synthetic construct
DAGTITGLNVLR

SEQ ID NO: 45 - protein, 10 aa, synthetic construct
EIAEAYLGGK

SEQ ID NO: 46 - protein, 10 aa, synthetic construct
LLGIETPLPK

SEQ ID NO: 47 - protein, 14 aa, synthetic construct
VTLNGVPAQPLGPR

SEQ ID NO: 48 - protein, 7 aa, synthetic construct
HGLYNLK

SEQ ID NO: 49 - protein, 10 aa, synthetic construct
ALSNVEGFER

SEQ ID NO: 50 - protein, 9 aa, synthetic construct
VAIVVGAPR

SEQ ID NO: 51 - protein, 16 aa, synthetic construct
SLPEGVANGIEVYSTK

SEQ ID NO: 52 - protein, 10 aa, synthetic construct
EVSFDVELPK

SEQ ID NO: 53 - protein, 13 aa, synthetic construct
IYSGILNLSDITK

SEQ ID NO: 54 - protein, 14 aa, synthetic construct
IAYGTQGSSGYSLR

SEQ ID NO: 55 - protein, 11 aa, synthetic construct
ITLPDFTGDLR

SEQ ID NO: 56 - protein, 11 aa, synthetic construct
LAEGFPLPLLK

SEQ ID NO: 57 - protein, 16 aa, synthetic construct
LIAPVAEEEATVPNNK

SEQ ID NO: 58 - protein, 8 aa, synthetic construct
DDEVAQLK

SEQ ID NO: 59 - protein, 13 aa, synthetic construct
LPPGLLANFTLLR

SEQ ID NO: 60 - protein, 10 aa, synthetic construct
DLLLPQPDLR

SEQ ID NO: 61 - protein, 11 aa, synthetic construct
EEAFLGITDEK

SEQ ID NO: 62 - protein, 8 aa, synthetic construct
WLTFSLGK

SEQ ID NO: 63 - protein, 15 aa, synthetic construct
AFALWSAVTPLTFTR

SEQ ID NO: 64 - protein, 9 aa, synthetic construct
AVIDDAFAR

SEQ ID NO: 65 - protein, 12 aa, synthetic construct
TGIAGGLWDVLK

SEQ ID NO: 66 - protein, 10 aa, synthetic construct
LITASGSYHK

SEQ ID NO: 67 - protein, 14 aa, synthetic construct
TGEAIVDAALSALR

SEQ ID NO: 68 - protein, 17 aa, synthetic construct
GSTAPVGGGAFPTIVER

SEQ ID NO: 69 - protein, 11 aa, synthetic construct
LVSLTLNLVTR

SEQ ID NO: 70 - protein, 14 aa, synthetic construct
VPGNVTAVLGETLK

SEQ ID NO: 71 - protein, 13 aa, synthetic construct
EESLDSDLYAELR

SEQ ID NO: 72 - protein, 8 aa, synthetic construct
TTSGIHPK

SEQ ID NO: 73 - protein, 9 aa, synthetic construct
VSFELFADK

SEQ ID NO: 74 - protein, 9 aa, synthetic construct
FEDENFILK

SEQ ID NO: 75 - protein, 9 aa, synthetic construct
LSEDYGVLK

SEQ ID NO: 76 - protein, 10 aa, synthetic construct
ATAVVDGAFK

SEQ ID NO: 77 - protein, 17 aa, synthetic construct
LIALSIDSVEDHLAWSK

SEQ ID NO: 78 - protein, 9 aa, synthetic construct
LPFPIIDDR

SEQ ID NO: 79 - protein, 11 aa, synthetic construct
AFLTLAEDILR

SEQ ID NO: 80 - protein, 10 aa, synthetic construct
FHTITTSYYR

SEQ ID NO: 81 - protein, 11 aa, synthetic construct
ALNSIIDVYHK

SEQ ID NO: 82 - protein, 7 aa, synthetic construct
GADVWFK

SEQ ID NO: 83 - protein, 15 aa, synthetic construct
GPGGVWAAEAISDAR

SEQ ID NO: 84 - protein, 20 aa, synthetic construct
FFGHGAEDSLADQAANEWGR

SEQ ID NO: 85 - protein, 11 aa, synthetic construct
AVVEVDESGTR

SEQ ID NO: 86 - protein, 10 aa, synthetic construct
EDQYHYLLDR

SEQ ID NO: 87 - protein, 15 aa, synthetic construct
FVGTPEVNQTTLYQR

SEQ ID NO: 88 - protein, 12 aa, synthetic construct
GFQALGDAADIR

SEQ ID NO: 89 - protein, 14 aa, synthetic construct
IQALQQQADEAEDR

SEQ ID NO: 90 - protein, 13 aa, synthetic construct
ASDAEGDVAALNR

SEQ ID NO: 91 - protein, 11 aa, synthetic construct
STVEGIQASVK

SEQ ID NO: 92 - protein, 9 aa, synthetic construct
IPTISTQLK

SEQ ID NO: 93 - protein, 9 aa, synthetic construct
DGTVTTDWK

SEQ ID NO: 94 - protein, 11 aa, synthetic construct
HIVTFDGQNFK

SEQ ID NO: 95 - protein, 14 aa, synthetic construct
AVTELNEPLSNEDR

SEQ ID NO: 96 - protein, 12 aa, synthetic construct
NSVVEASEAAYK

SEQ ID NO: 97 - protein, 14 aa, synthetic construct
SVTEQGAELSNEER

SEQ ID NO: 98 - protein, 12 aa, synthetic construct
FLIPNASQAESK

SEQ ID NO: 99 - protein, 8 aa, synthetic construct
LLELTGPK

SEQ ID NO: 100 - protein, 7 aa, synthetic construct
SSTSPDR

SEQ ID NO: 101 - protein, 11 aa, synthetic construct
ATWSGAVLAGR

SEQ ID NO: 102 - protein, 9 aa, synthetic construct
ISASAEELR

SEQ ID NO: 103 - protein, 9 aa, synthetic construct
LAPLAEDVR

SEQ ID NO: 104 - protein, 7 aa, synthetic construct
DYWSTVK

SEQ ID NO: 105 - protein, 7 aa, synthetic construct
NEADELR

SEQ ID NO: 106 - protein, 8 aa, synthetic construct
ALADGVQK

SEQ ID NO: 107 - protein, 8 aa, synthetic construct
ELSDIAHR

SEQ ID NO: 108 - protein, 13 aa, synthetic construct
NWGEGWGFVPSDR

SEQ ID NO: 109 - protein, 8 aa, synthetic construct
VDADADPR

SEQ ID NO: 110 - protein, 8 aa, synthetic construct
ELTLEDLK

SEQ ID NO: 111 - protein, 7 aa, synthetic construct
GADLSLR

SEQ ID NO: 112 - protein, 10 aa, synthetic construct
GYSIFSYATK

SEQ ID NO: 113 - protein, 11 aa, synthetic construct
AVVFLEPQWYR

SEQ ID NO: 114 - protein, 12 aa, synthetic construct
YFHHNSDFYIPK

SEQ ID NO: 115 - protein, 12 aa, synthetic construct
TTDVTQTFGIEK

SEQ ID NO: 116 - protein, 14 aa, synthetic construct
STGGAPTFNVTVTK

SEQ ID NO: 117 - protein, 11 aa, synthetic construct
DLATVYVDVLK

SEQ ID NO: 118 - protein, 7 aa, synthetic construct
AHVDALR

SEQ ID NO: 119 - protein, 11 aa, synthetic construct
ATEHLSTLSEK

SEQ ID NO: 120 - protein, 9 aa, synthetic construct
VVEESELAR

SEQ ID NO: 121 - protein, 7 aa, synthetic construct
GEIHLGR

SEQ ID NO: 122 - protein, 8 aa, synthetic construct
ALQASALK

SEQ ID NO: 123 - protein, 7 aa, synthetic construct
DYAEVGR

SEQ ID NO: 124 - protein, 8 aa, synthetic construct
TLPAAAFR

SEQ ID NO: 125 - protein, 8 aa, synthetic construct
VSPAAPER

SEQ ID NO: 126 - protein, 11 aa, synthetic construct
ITLLSALVETR

SEQ ID NO: 127 - protein, 10 aa, synthetic construct
AIHLDLEEYR

SEQ ID NO: 128 - protein, 8 aa, synthetic construct
HLEDVFSK

SEQ ID NO: 129 - protein, 9 aa, synthetic construct
DGSTIPIAK

SEQ ID NO: 130 - protein, 9 aa, synthetic construct
AFTPFSGPK

SEQ ID NO: 131 - protein, 11 aa, synthetic construct
ALGTLLHTNLR

SEQ ID NO: 132 - protein, 9 aa, synthetic construct
YEYLEGGDR

SEQ ID NO: 133 - protein, 7 aa, synthetic construct
IEPSQAK

SEQ ID NO: 134 - protein, 7 aa, synthetic construct
FHPLHSK

SEQ ID NO: 135 - protein, 8 aa, synthetic construct
FLNVLSPR

SEQ ID NO: 136 - protein, 11 aa, synthetic construct
YGQPLPGYTTK

SEQ ID NO: 137 - protein, 11 aa, synthetic construct
ITPTITNFALR

SEQ ID NO: 138 - protein, 7 aa, synthetic construct
VLDSGFR

SEQ ID NO: 139 - protein, 7 aa, synthetic construct
STVEIFK

SEQ ID NO: 140 - protein, 9 aa, synthetic construct
ILIGTVFHK

SEQ ID NO: 141 - protein, 8 aa, synthetic construct
AEIEYLEK

SEQ ID NO: 142 - protein, 8 aa, synthetic construct
SYYWIGIR

SEQ ID NO: 143 - protein, 8 aa, synthetic construct
DFTFDLYR

SEQ ID NO: 144 - protein, 9 aa, synthetic construct
EYAPGETVK

SEQ ID NO: 145 - protein, 8 aa, synthetic construct
TDEGIAYR

SEQ ID NO: 146 - protein, 8 aa, synthetic construct
GLFIIDGK

SEQ ID NO: 147 - protein, 10 aa, synthetic construct
ADLSGITGAR

SEQ ID NO: 148 - protein, 9 aa, synthetic construct
GQTLLAVAK

SEQ ID NO: 149 - protein, 9 aa, synthetic construct
YEVTVVSVR

SEQ ID NO: 150 - protein, 8 aa, synthetic construct
TASDFITK

SEQ ID NO: 151 - protein, 7 aa, synthetic construct
AVEVLPK

SEQ ID NO: 152 - protein, 8 aa, synthetic construct
TGAQELLR

SEQ ID NO: 153 - protein, 12 aa, synthetic construct
LDTLAQEVALLK

SEQ ID NO: 154 - protein, 14 aa, synthetic construct
NWETEITAQPDGGK

SEQ ID NO: 155 - protein, 10 aa, synthetic construct
WDEELAAFAK

SEQ ID NO: 156 - protein, 7 aa, synthetic construct
GGVVGIK

SEQ ID NO: 157 - protein, 11 aa, synthetic construct
LTGLLDLALEK

SEQ ID NO: 158 - protein, 15 aa, synthetic construct
VLLVVALLALLASAR

SEQ ID NO: 159 - protein, 7 aa, synthetic construct
AEEAGAR

SEQ ID NO: 160 - protein, 7 aa, synthetic construct
QYADTVK

SEQ ID NO: 161 - protein, 9 aa, synthetic construct
EAILSHTEK

SEQ ID NO: 162 - protein, 7 aa, synthetic construct
LTSEASR

SEQ ID NO: 163 - protein, 11 aa, synthetic construct
QDNEILIFWSK

SEQ ID NO: 164 - protein, 11 aa, synthetic construct
TFLTVYWTPER

SEQ ID NO: 165 - protein, 8 aa, synthetic construct
ATPEDNGR

SEQ ID NO: 166 - protein, 11 aa, synthetic construct
DSDLLSPSDFK

SEQ ID NO: 167 - protein, 10 aa, synthetic construct
FGYILHTDNR

SEQ ID NO: 168 - protein, 9 aa, synthetic construct
EETGQVLER

SEQ ID NO: 169 - protein, 7 aa, synthetic construct
EAGGGYR

SEQ ID NO: 170 - protein, 8 aa, synthetic construct
VFLEIDNR

SEQ ID NO: 171 - protein, 7 aa, synthetic construct
VTDLDAR

SEQ ID NO: 172 - protein, 7 aa, synthetic construct
ISSEAHR

SEQ ID NO: 173 - protein, 10 aa, synthetic construct
VPLALFALNR

SEQ ID NO: 174 - protein, 10 aa, synthetic construct
EGVHGGLINK

SEQ ID NO: 175 - protein, 9 aa, synthetic construct
QESFFVDER

SEQ ID NO: 176 - protein, 7 aa, synthetic construct
VVNPVAG

SEQ ID NO: 177 - protein, 9 aa, synthetic construct
VGNSLFLDK

SEQ ID NO: 178 - protein, 8 aa, synthetic construct
ADAAPDEK

SEQ ID NO: 179 - protein, 10 aa, synthetic construct
LQGSFDVSVK

SEQ ID NO: 180 - protein, 7 aa, synthetic construct
ENGTISR

SEQ ID NO: 181 - protein, 10 aa, synthetic construct
NWGLSVYADK

SEQ ID NO: 182 - protein, 7 aa, synthetic construct
DLWNIFK

SEQ ID NO: 183 - protein, 9 aa, synthetic construct
LEILEELVK

SEQ ID NO: 184 - protein, 9 aa, synthetic construct
LGELTVTDR

SEQ ID NO: 185 - protein, 8 aa, synthetic construct
ATEASDSR

SEQ ID NO: 186 - protein, 8 aa, synthetic construct
STHVPIPK

SEQ ID NO: 187 - protein, 7 aa, synthetic construct
AAFNSGK

SEQ ID NO: 188 - protein, 8 aa, synthetic construct
TVDGPSGK

SEQ ID NO: 189 - protein, 9 aa, synthetic construct
SIAPGAFDR

SEQ ID NO: 190 - protein, 9 aa, synthetic construct
HHLGLEEPK

SEQ ID NO: 191 - protein, 7 aa, synthetic construct
HNGTGGK

SEQ ID NO: 192 - protein, 9 aa, synthetic construct
SEEFLIAGK

SEQ ID NO: 193 - protein, 7 aa, synthetic construct
FGVSTGR

SEQ ID NO: 194 - protein, 7 aa, synthetic construct
APDVDIK

SEQ ID NO: 195 - protein, 7 aa, synthetic construct
GPEVDIK

SEQ ID NO: 196 - protein, 11 aa, synthetic construct
LDGLVPSYAEK

SEQ ID NO: 197 - protein, 7 aa, synthetic construct
GGFQALK

SEQ ID NO: 198 - protein, 8 aa, synthetic construct
GSTEEGAR

SEQ ID NO: 199 - protein, 8 aa, synthetic construct
LLGTVYEK

SEQ ID NO: 200 - protein, 10 aa, synthetic construct
LFITPEDLSK

SEQ ID NO: 201 - protein, 8 aa, synthetic construct
LVSEIVAK

SEQ ID NO: 202 - protein, 7 aa, synthetic construct
EYLHFVK

SEQ ID NO: 203 - protein, 10 aa, synthetic construct
LSFSSTGSTR

SEQ ID NO: 204 - protein, 10 aa, synthetic construct
LVFIGDTLTR

SEQ ID NO: 205 - protein, 10 aa, synthetic construct
GSEIVAGLEK

SEQ ID NO: 206 - protein, 7 aa, synthetic construct
WPTTLSR

SEQ ID NO: 207 - protein, 7 aa, synthetic construct
SPHVGVK

SEQ ID NO: 208 - protein, 7 aa, synthetic construct
VETADLK

SEQ ID NO: 209 - protein, 9 aa, synthetic construct
VLGLETEHK

SEQ ID NO: 210 - protein, 11 aa, synthetic construct
HAADLGALETR

SEQ ID NO: 211 - protein, 11 aa, synthetic construct
KPITVLEILQK

SEQ ID NO: 212 - protein, 11 aa, synthetic construct
TDSSPGPSQIK

SEQ ID NO: 213 - protein, 10 aa, synthetic construct
LGEASDFIVR

SEQ ID NO: 214 - protein, 15 aa, synthetic construct
ADEPSTEESDLEIDK

SEQ ID NO: 215 - protein, 14 aa, synthetic construct
VAAIEVLNDGELQK

SEQ ID NO: 216 - protein, 7 aa, synthetic construct
AQEEQER

SEQ ID NO: 217 - protein, 11 aa, synthetic construct
YLAEFATGNDR

SEQ ID NO: 218 - protein, 8 aa, synthetic construct
EDLVYQAK

SEQ ID NO: 219 - protein, 8 aa, synthetic construct
IISSIEQK

SEQ ID NO: 220 - protein, 8 aa, synthetic construct
EISDHVLR

SEQ ID NO: 221 - protein, 7 aa, synthetic construct
TLETLIR

SEQ ID NO: 222 - protein, 7 aa, synthetic construct
LATAHAK